SEQUENCE LISTING (68 sequences)

SEQ ID NO: 1 - protein, 328 aa, Unknown
  Mycobacterium species
VVDAHRGGHPTPMSSTKATLRLAEATDSSGKITKRGADKLISTIDEFAKIAISSGCAELMAFATSAVRDAENSEDVLSRVRKETGVELQALRGEDESRLTFLAVRRWYGWSAGRILNLDIGGGSLEVSSGVDEEPEIALSLPLGAGRLTREWLPDDPPGRRRVAMLRDWLDAELAEPSVTVLEAGSPDLAVATSKTFRSLARLTGAAPSMAGPRVKRTLTANGLRQLIAFISRMTAVDRAELEGVSADRAPQIVAGALVAEASMRALSIEAVEICPWALREGLILRKLDSEADGTALIESSSVHTSVRAVGGQPADRNAANRSRGSKP

SEQ ID NO: 2 - protein, 327 aa, Unknown
  Mycobacterium species
VDAHRGGHPTPMSSTKATLRLAEATDSSGKITKRGADKLISTIDEFAKIAISSGCAELMAFATSAVRDAENSEDVLSRVRKETGVELQALRGEDESRLTFLAVRRWYGWSAGRILNLDIGGGSLEVSSGVDEEPEIALSLPLGAGRLTREWLPDDPPGRRRVAMLRDWLDAELAEPSVTVLEAGSPDLAVATSKTFRSLARLTGAAPSMAGPRVKRTLTANGLRQLIAFISRMTAVDRAELEGVSADRAPQIVAGALVAEASMRALSIEAVEICPWALREGLILRKLDSEADGTALIESSSVHTSVRAVGGQPADRNAANRSRGSKP

SEQ ID NO: 3 - protein, 143 aa, Unknown
  Mycobacterium species
MITNLRRRTAMAAAGLGAALGLGILLVPTVDAHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACN

SEQ ID NO: 4 - protein, 111 aa, Unknown
  Mycobacterium species
HLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACN

SEQ ID NO: 5 - protein, 325 aa, Unknown
  Mycobacterium species
MTDVSRKIRAWGRRLMIGTAAAVVLPGLVGLAGGAATAGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 6 - protein, 285 aa, Unknown
  Mycobacterium species
FSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 7 - protein, 154 aa, Unknown
  Mycobacterium species
MTPGLLTTAGAGRPRDRCARIVCTVFIETAVVATMFVALLGLSTISSKADDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDD

SEQ ID NO: 8 - protein, 105 aa, Unknown
  Mycobacterium species
DDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDD

SEQ ID NO: 9 - protein, 580 aa, Unknown
  Mycobacterium species
MNFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLF

SEQ ID NO: 10 - protein, 579 aa, Unknown
  Mycobacterium species
NFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLF

SEQ ID NO: 11 - protein, 193 aa, Unknown
  Mycobacterium species
MKVKNTIAATSFAAAGLAALAVAVSPPAAAGDLVGPGCAEYAAANPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASVTVTGQGNSLKVGNADVVCGGVSTANATVYMIDSVLMPPA

SEQ ID NO: 12 - protein, 163 aa, Unknown
  Mycobacterium species
GDLVGPGCAEYAAANPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASVTVTGQGNSLKVGNADVVCGGVSTANATVYMIDSVLMPPA

SEQ ID NO: 13 - protein, 193 aa, Unknown
  Mycobacterium species
MKVKNTIAATSFAAAGLAALAVAVSPPAAAGDLVSPGCAEYAAANPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASVTVTGQGNSLKVGNADVVCGGVSTANATVYMIDSVLMPPA

SEQ ID NO: 14 - protein, 163 aa, Unknown
  Mycobacterium species
GDLVSPGCAEYAAANPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASVTVTGQGNSLKVGNADVVCGGVSTANATVYMIDSVLMPPA

SEQ ID NO: 15 - protein, 393 aa, Unknown
  Mycobacterium species
VVDFGALPPEINSARMYAGPGSASLVAAAKMWDSVASDLFSAASAFQSVVWGLTVGSWIGSSAGLMAAAASPYVAWMSVTAGQAQLTAAQVRVAAAAYETAYRLTVPPPVIAENRTELMTLTATNLLGQNTPAIEANQAAYSQMWGQDAEAMYGYAATAATATEALLPFEDAPLITNPGGLLEQAVAVEEAIDTAAANQLMNNVPQALQQLAQPAQGVVPSSKLGGLWTAVSPHLSPLSNVSSIANNHMSMMGTGVSMTNTLHSMLKGLAPAAAQAVETAAENGVWAMSSLGSQLGSSLGSSGLGAGVAANLGRAASVGSLSVPPAWAAANQAVTPAARALPLTSLTSAAQTAPGHMLGGLPLGHSVNAGSGINNALRVPARAYAIPRTPAAG

SEQ ID NO: 16 - protein, 180 aa, Unknown
  Mycobacterium species
VVDFGALPPEINSARMYAGPGSASLVAAAKMWDSVASDLFSAASAFQSVVWGLTVGSWIGSSAGLMAAAASPYVAWMSVTAGQAQLTAAQVRVAAAAYETAYRLTVPPPVIAENRTELMTLTATNLLGQNTPAIEANQAAYSQMWGQDAEAMYGYAATAATATEALLPFEDAPLITNPGG

SEQ ID NO: 17 - protein, 94 aa, Unknown
  Mycobacterium species
MTINYQFGDVDAHGAMIRAQAGSLEAEHQAIISDVLTASDFWGGAGSAACQGFITQLGRNFQVIYEQANAHGQKVQAAGNNMAQTDSAVGSSWA

SEQ ID NO: 18 - protein, 98 aa, Unknown
  Mycobacterium species
MTSRFMTDPHAMRDMAGRFEVHAQTVEDEARRMWASAQNISGAGWSGMAEATSLDTMTQMNQAFRNIVNMLHGVRDGLVRDANNYEQQEQASQQILSS

SEQ ID NO: 19 - protein, 284 aa, Unknown
  Mycobacterium species
VPNRRRRKLSTAMSAVAALAVASPCAYFLVYESTETTERPEHHEFKQAAVLTDLPGELMSALSQGLSQFGINIPPVPSLTGSGDASTGLTGPGLTSPGLTSPGLTSPGLTDPALTSPGLTPTLPGSLAAPGTTLAPTPGVGANPALTNPALTSPTGATPGLTSPTGLDPALGGANEIPITTPVGLDPGADGTYPILGDPTLGTIPSSPATTSTGGGGLVNDVMQVANELGASQAIDLLKGVLMPSIMQAVQNGGAAAPAASPPVPPIPAAAAVPPTDPITVPVA

SEQ ID NO: 20 - protein, 262 aa, Unknown
  Mycobacterium species
SPCAYFLVYESTETTERPEHHEFKQAAVLTDLPGELMSALSQGLSQFGINIPPVPSLTGSGDASTGLTGPGLTSPGLTSPGLTSPGLTDPALTSPGLTPTLPGSLAAPGTTLAPTPGVGANPALTNPALTSPTGATPGLTSPTGLDPALGGANEIPITTPVGLDPGADGTYPILGDPTLGTIPSSPATTSTGGGGLVNDVMQVANELGASQAIDLLKGVLMPSIMQAVQNGGAAAPAASPPVPPIPAAAAVPPTDPITVPVA

SEQ ID NO: 21 - protein, 543 aa, Artificial Sequence
  Synthetic Construct
HLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDVVDAHRGGHPTPMSSTKATLRLAEATDSSGKITKRGADKLISTIDEFAKIAISSGCAELMAFATSAVRDAENSEDVLSRVRKETGVELQALRGEDESRLTFLAVRRWYGWSAGRILNLDIGGGSLEVSSGVDEEPEIALSLPLGAGRLTREWLPDDPPGRRRVAMLRDWLDAELAEPSVTVLEAGSPDLAVATSKTFRSLARLTGAAPSMAGPRVKRTLTANGLRQLIAFISRMTAVDRAELEGVSADRAPQIVAGALVAEASMRALSIEAVEICPWALREGLILRKLDSEADGTALIESSSVHTSVRAVGGQPADRNAANRSRGSKPST

SEQ ID NO: 22 - protein, 650 aa, Artificial Sequence
  Synthetic Construct
DDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDDGTHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDMVDAHRGGHPTPMSSTKATLRLAEATDSSGKITKRGADKLISTIDEFAKIAISSGCAELMAFATSAVRDAENSEDVLSRVRKETGVELQALRGEDESRLTFLAVRRWYGWSAGRILNLDIGGGSLEVSSGVDEEPEIALSLPLGAGRLTREWLPDDPPGRRRVAMLRDWLDAELAEPSVTVLEAGSPDLAVATSKTFRSLARLTGAAPSMAGPRVKRTLTANGLRQLIAFISRMTAVDRAELEGVSADRAPQIVAGALVAEASMRALSIEAVEICPWALREGLILRKLDSEADGTALIESSSVHTSVRAVGGQPADRNAANRSRGSKPST

SEQ ID NO: 23 - protein, 672 aa, Artificial Sequence
  Synthetic Construct
HMMTINYQFGDVDAHGAMIRAQAGSLEAEHQAIISDVLTASDFWGGAGSAACQGFITQLGRNFQVIYEQANAHGQKVQAAGNNMAQTDSAVGSSWAGTDDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDDGSVVDFGALPPEINSARMYAGPGSASLVAAAKMWDSVASDLFSAASAFQSVVWGLTVGSWIGSSAGLMAAAASPYVAWMSVTAGQAQLTAAQVRVAAAAYETAYRLTVPPPVIAENRTELMTLTATNLLGQNTPAIEANQAAYSQMWGQDAEAMYGYAATAATATEALLPFEDAPLITNPGGEFFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 24 - protein, 795 aa, Artificial Sequence
  Synthetic Construct
HLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDINFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLFST

SEQ ID NO: 25 - protein, 795 aa, Artificial Sequence
  Synthetic Construct
HLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDMNFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLFST

SEQ ID NO: 26 - protein, 846 aa, Artificial Sequence
  Synthetic Construct
MGDLVSPGCAEYAAANPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASVTVTGQGNSLKVGNADVVCGGVSTANATVYMIDSVLMPPAGSVVDFGALPPEINSARMYAGPGSASLVAAAKMWDSVASDLFSAASAFQSVVWGLTVGSWIGSSAGLMAAAASPYVAWMSVTAGQAQLTAAQVRVAAAAYETAYRLTVPPPVIAENRTELMTLTATNLLGQNTPAIEANQAAYSQMWGQDAEAMYGYAATAATATEALLPFEDAPLITNPGGLLEQAVAVEEAIDTAAANQLMNNVPQALQQLAQPAQGVVPSSKLGGLWTAVSPHLSPLSNVSSIANNHMSMMGTGVSMTNTLHSMLKGLAPAAAQAVETAAENGVWAMSSLGSQLGSSLGSSGLGAGVAANLGRAASVGSLSVPPAWAAANQAVTPAARALPLTSLTSAAQTAPGHMLGGLPLGHSVNAGSGINNALRVPARAYAIPRTPAAGEFFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 27 - protein, 883 aa, Artificial Sequence
  Synthetic Construct
MTINYQFGDVDAHGAMIRAQAGSLEAEHQAIISDVLTASDFWGGAGSAACQGFITQLGRNFQVIYEQANAHGQKVQAAGNNMAQTDSAVGSSWAGTDDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDDGSVVDFGALPPEINSARMYAGPGSASLVAAAKMWDSVASDLFSAASAFQSVVWGLTVGSWIGSSAGLMAAAASPYVAWMSVTAGQAQLTAAQVRVAAAAYETAYRLTVPPPVIAENRTELMTLTATNLLGQNTPAIEANQAAYSQMWGQDAEAMYGYAATAATATEALLPFEDAPLITNPGGLLEQAVAVEEAIDTAAANQLMNNVPQALQQLAQPAQGVVPSSKLGGLWTAVSPHLSPLSNVSSIANNHMSMMGTGVSMTNTLHSMLKGLAPAAAQAVETAAENGVWAMSSLGSQLGSSLGSSGLGAGVAANLGRAASVGSLSVPPAWAAANQAVTPAARALPLTSLTSAAQTAPGHMLGGLPLGHSVNAGSGINNALRVPARAYAIPRTPAAGEFFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 28 - protein, 891 aa, Artificial Sequence
  Synthetic Construct
MTINYQFGDVDAHGAMIRAQAGSLEAEHQAIISDVLTASDFWGGAGSAACQGFITQLGRNFQVIYEQANAHGQKVQAAGNNMAQTDSAVGSSWAGTHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDINFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLFST

SEQ ID NO: 29 - protein, 891 aa, Artificial Sequence
  Synthetic Construct
MTINYQFGDVDAHGAMIRAQAGSLEAEHQAIISDVLTASDFWGGAGSAACQGFITQLGRNFQVIYEQANAHGQKVQAAGNNMAQTDSAVGSSWAGTHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDMNFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLFST

SEQ ID NO: 30 - protein, 902 aa, Artificial Sequence
  Synthetic Construct
DDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDDGTHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDINFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLFST

SEQ ID NO: 31 - protein, 902 aa, Artificial Sequence
  Synthetic Construct
DDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDDGTHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDMNFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLFST

SEQ ID NO: 32 - protein, 914 aa, Artificial Sequence
  Synthetic Construct
DDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDDELSPCAYFLVYESTETTERPEHHEFKQAAVLTDLPGELMSALSQGLSQFGINIPPVPSLTGSGDASTGLTGPGLTSPGLTSPGLTSPGLTDPALTSPGLTPTLPGSLAAPGTTLAPTPGVGANPALTNPALTSPTGATPGLTSPTGLDPALGGANEIPITTPVGLDPGADGTYPILGDPTLGTIPSSPATTSTGGGGLVNDVMQVANELGASQAIDLLKGVLMPSIMQAVQNGGAAAPAASPPVPPIPAAAAVPPTDPITVPVAGTHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDMVDAHRGGHPTPMSSTKATLRLAEATDSSGKITKRGADKLISTIDEFAKIAISSGCAELMAFATSAVRDAENSEDVLSRVRKETGVELQALRGEDESRLTFLAVRRWYGWSAGRILNLDIGGGSLEVSSGVDEEPEIALSLPLGAGRLTREWLPDDPPGRRRVAMLRDWLDAELAEPSVTVLEAGSPDLAVATSKTFRSLARLTGAAPSMAGPRVKRTLTANGLRQLIAFISRMTAVDRAELEGVSADRAPQIVAGALVAEASMRALSIEAVEICPWALREGLILRKLDSEADGTALIESSSVHTSVRAVGGQPADRNAANRSRGSKPST

SEQ ID NO: 33 - protein, 942 aa, Artificial Sequence
  Synthetic Construct
MTINYQFGDVDAHGAMIRAQAGSLEAEHQAIISDVLTASDFWGGAGSAACQGFITQLGRNFQVIYEQANAHGQKVQAAGNNMAQTDSAVGSSWAGTMGDLVSPGCAEYAAANPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASVTVTGQGNSLKVGNADVVCGGVSTANATVYMIDSVLMPPAGSVVDFGALPPEINSARMYAGPGSASLVAAAKMWDSVASDLFSAASAFQSVVWGLTVGSWIGSSAGLMAAAASPYVAWMSVTAGQAQLTAAQVRVAAAAYETAYRLTVPPPVIAENRTELMTLTATNLLGQNTPAIEANQAAYSQMWGQDAEAMYGYAATAATATEALLPFEDAPLITNPGGLLEQAVAVEEAIDTAAANQLMNNVPQALQQLAQPAQGVVPSSKLGGLWTAVSPHLSPLSNVSSIANNHMSMMGTGVSMTNTLHSMLKGLAPAAAQAVETAAENGVWAMSSLGSQLGSSLGSSGLGAGVAANLGRAASVGSLSVPPAWAAANQAVTPAARALPLTSLTSAAQTAPGHMLGGLPLGHSVNAGSGINNALRVPARAYAIPRTPAAGEFFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 34 - protein, 1082 aa, Artificial Sequence
  Synthetic Construct
GTHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDMNFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLFSTFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 35 - protein, 1166 aa, Artificial Sequence
  Synthetic Construct
DDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDDELSPCAYFLVYESTETTERPEHHEFKQAAVLTDLPGELMSALSQGLSQFGINIPPVPSLTGSGDASTGLTGPGLTSPGLTSPGLTSPGLTDPALTSPGLTPTLPGSLAAPGTTLAPTPGVGANPALTNPALTSPTGATPGLTSPTGLDPALGGANEIPITTPVGLDPGADGTYPILGDPTLGTIPSSPATTSTGGGGLVNDVMQVANELGASQAIDLLKGVLMPSIMQAVQNGGAAAPAASPPVPPIPAAAAVPPTDPITVPVAGTHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDINFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLFST

SEQ ID NO: 36 - protein, 1166 aa, Artificial Sequence
  Synthetic Construct
DDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDDELSPCAYFLVYESTETTERPEHHEFKQAAVLTDLPGELMSALSQGLSQFGINIPPVPSLTGSGDASTGLTGPGLTSPGLTSPGLTSPGLTDPALTSPGLTPTLPGSLAAPGTTLAPTPGVGANPALTNPALTSPTGATPGLTSPTGLDPALGGANEIPITTPVGLDPGADGTYPILGDPTLGTIPSSPATTSTGGGGLVNDVMQVANELGASQAIDLLKGVLMPSIMQAVQNGGAAAPAASPPVPPIPAAAAVPPTDPITVPVAGTHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDMNFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLFST

SEQ ID NO: 37 - protein, 1176 aa, Artificial Sequence
  Synthetic Construct
MTINYQFGDVDAHGAMIRAQAGSLEAEHQAIISDVLTASDFWGGAGSAACQGFITQLGRNFQVIYEQANAHGQKVQAAGNNMAQTDSAVGSSWAGTHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDINFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLFSTFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 38 - protein, 1176 aa, Artificial Sequence
  Synthetic Construct
MTINYQFGDVDAHGAMIRAQAGSLEAEHQAIISDVLTASDFWGGAGSAACQGFITQLGRNFQVIYEQANAHGQKVQAAGNNMAQTDSAVGSSWAGTHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDMNFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLFSTFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 39 - DNA, 981 bp, Mycobacterium tuberculosis
gtcgatgcccaccgcggcggccacccgaccccgatgagctcgacgaaggccacgctgcggctggccgaggccaccgacagctcgggcaagatcaccaagcgcggagccgacaagctgatttccaccatcgacgaattcgccaagattgccatcagctcgggctgtgccgagctgatggccttcgccacgtcggcggtccgcgacgccgagaattccgaggacgtcctgtcccgggtgcgcaaagagaccggtgtcgagttgcaggcgctgcgtggggaggacgagtcacggctgaccttcctggccgtgcgacgatggtacgggtggagcgctgggcgcatcctcaacctcgacatcggcggcggctcgctggaagtgtccagtggcgtggacgaggagcccgagattgcgttatcgctgcccctgggcgccggacggttgacccgagagtggctgcccgacgatccgccgggccggcgccgggtggcgatgctgcgagactggctggatgccgagctggccgagcccagtgtgaccgtcctggaagccggcagccccgacctggcggtcgcaacgtcgaagacgtttcgctcgttggcgcgactaaccggtgcggccccatccatggccgggccgcgggtgaagaggaccctaacggcaaatggtctgcggcaactcatcgcgtttatctctaggatgacggcggttgaccgtgcagaactggaaggggtaagcgccgaccgagcgccgcagattgtggccggcgccctggtggcagaggcgagcatgcgagcactgtcgatagaagcggtggaaatctgcccgtgggcgctgcgggaaggtctcatcttgcgcaaactcgacagcgaagccgacggaaccgccctcatcgagtcttcgtctgtgcacacttcggtgcgtgccgtcggaggtcagccagctgatcggaacgcggccaaccgatcgagaggcagcaaacca

SEQ ID NO: 40 - DNA, 332 bp, Mycobacterium tuberculosis
catctcgccaacggttcgatgtcggaagtcatgatgtcggaaattgccgggttgcctatccctccgattatccattacggggcgattgcctatgcccccagcggcgcgtcgggcaaagcgtggcaccagcgcacaccggcgcgagcagagcaagtcgcactagaaaagtgcggtgacaagacttgcaaagtggttagtcgcttcaccaggtgcggcgcggtcgcctacaacggctcgaaataccaaggcggaaccggactcacgcgccgcgcggcagaagacgacgccgtgaaccgactcgaaggcgggcggatcgtcaactgggcgtgcaa

SEQ ID NO: 41 - DNA, 855 bp, Mycobacterium tuberculosis
ttctcccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccttcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatggggcctagcctgatcggcctcgcgatgggtgacgccggcggttacaaggccgcagacatgtggggtccctcgagtgacccggcatgggagcgcaacgaccctacgcagcagatccccaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaacgccgcgggcgggcacaacgccgtgttcaacttcccgcccaacggcacgcacagctgggagtactggggcgctcagctcaacgccatgaagggtgacctgcagagttcgttaggcgccggc

SEQ ID NO: 42 - DNA, 1740 bp, Mycobacterium tuberculosis
atgaatttcgccgttttgccgccggaggtgaattcggcgcgcatattcgccggtgcgggcctgggcccaatgctggcggcggcgtcggcctgggacgggttggccgaggagttgcatgccgcggcgggctcgttcgcgtcggtgaccaccgggttggcgggcgacgcgtggcatggtccggcgtcgctggcgatgacccgcgcggccagcccgtatgtggggtggttgaacacggcggcgggtcaggccgcgcaggcggccggccaggcgcggctagcggcgagcgcgttcgaggcgacgctggcggccaccgtgtctccagcgatggtcgcggccaaccggacacggctggcgtcgctggtggcagccaacttgctgggccagaacgccccggcgatcgcggccgcggaggctgaatacgagcagatatgggcccaggacgtggccgcgatgttcggctatcactccgccgcgtcggcggtggccacgcagctggcgcctattcaagagggtttgcagcagcagctgcaaaacgtgctggcccagttggctagcgggaacctgggcagcggaaatgtgggcgtcggcaacatcggcaacgacaacattggcaacgcaaacatcggcttcggaaatcgaggcgacgccaacatcggcatcgggaatatcggcgacagaaacctcggcattgggaacaccggcaattggaatatcggcatcggcatcaccggcaacggacaaatcggcttcggcaagcctgccaaccccgacgtcttggtggtgggcaacggcggcccgggagtaaccgcgttggtcatgggcggcaccgacagcctactgccgctgcccaacatccccttactcgagtacgctgcgcggttcatcacccccgtgcatcccggatacaccgctacgttcctggaaacgccatcgcagtttttcccattcaccgggctgaatagcctgacctatgacgtctccgtggcccagggcgtaacgaatctgcacaccgcgatcatggcgcaactcgcggcgggaaacgaagtcgtcgtcttcggcacctcccaaagcgccacgatagccaccttcgaaatgcgctatctgcaatccctgccagcacacctgcgtccgggtctcgacgaattgtcctttacgttgaccggcaatcccaaccggcccgacggtggcattcttacgcgttttggcttctccataccgcagttgggtttcacattgtccggcgcgacgcccgccgacgcctaccccaccgtcgattacgcgttccagtacgacggcgtcaacgacttccccaaatacccgctgaatgtcttcgcgaccgccaacgcgatcgcgggcatccttttcctgcactccgggttgattgcgttgccgcccgatcttgcctcgggcgtggttcaaccggtgtcctcaccggacgtcctgaccacctacatcctgctgcccagccaagatctgccgctgctggtcccgctgcgtgctatccccctgctgggaaacccgcttgccgacctcatccagccggacttgcgggtgctcgtcgagttgggttatgaccgcaccgcccaccaggacgtgcccagcccgttcggactgtttccggacgtcgattgggccgaggtggccgcggacctgcagcaaggcgccgtgcaaggcgtcaacgacgccctgtccggactggggctgccgccgccgtggcagccggcgctaccccgacttttc

SEQ ID NO: 43 - DNA, 1737 bp, Mycobacterium tuberculosis
aatttcgccgttttgccgccggaggtgaattcggcgcgcatattcgccggtgcgggcctgggcccaatgctggcggcggcgtcggcctgggacgggttggccgaggagttgcatgccgcggcgggctcgttcgcgtcggtgaccaccgggttggcgggcgacgcgtggcatggtccggcgtcgctggcgatgacccgcgcggccagcccgtatgtggggtggttgaacacggcggcgggtcaggccgcgcaggcggccggccaggcgcggctagcggcgagcgcgttcgaggcgacgctggcggccaccgtgtctccagcgatggtcgcggccaaccggacacggctggcgtcgctggtggcagccaacttgctgggccagaacgccccggcgatcgcggccgcggaggctgaatacgagcagatatgggcccaggacgtggccgcgatgttcggctatcactccgccgcgtcggcggtggccacgcagctggcgcctattcaagagggtttgcagcagcagctgcaaaacgtgctggcccagttggctagcgggaacctgggcagcggaaatgtgggcgtcggcaacatcggcaacgacaacattggcaacgcaaacatcggcttcggaaatcgaggcgacgccaacatcggcatcgggaatatcggcgacagaaacctcggcattgggaacaccggcaattggaatatcggcatcggcatcaccggcaacggacaaatcggcttcggcaagcctgccaaccccgacgtcttggtggtgggcaacggcggcccgggagtaaccgcgttggtcatgggcggcaccgacagcctactgccgctgcccaacatccccttactcgagtacgctgcgcggttcatcacccccgtgcatcccggatacaccgctacgttcctggaaacgccatcgcagtttttcccattcaccgggctgaatagcctgacctatgacgtctccgtggcccagggcgtaacgaatctgcacaccgcgatcatggcgcaactcgcggcgggaaacgaagtcgtcgtcttcggcacctcccaaagcgccacgatagccaccttcgaaatgcgctatctgcaatccctgccagcacacctgcgtccgggtctcgacgaattgtcctttacgttgaccggcaatcccaaccggcccgacggtggcattcttacgcgttttggcttctccataccgcagttgggtttcacattgtccggcgcgacgcccgccgacgcctaccccaccgtcgattacgcgttccagtacgacggcgtcaacgacttccccaaatacccgctgaatgtcttcgcgaccgccaacgcgatcgcgggcatccttttcctgcactccgggttgattgcgttgccgcccgatcttgcctcgggcgtggttcaaccggtgtcctcaccggacgtcctgaccacctacatcctgctgcccagccaagatctgccgctgctggtcccgctgcgtgctatccccctgctgggaaacccgcttgccgacctcatccagccggacttgcgggtgctcgtcgagttgggttatgaccgcaccgcccaccaggacgtgcccagcccgttcggactgtttccggacgtcgattgggccgaggtggccgcggacctgcagcaaggcgccgtgcaaggcgtcaacgacgccctgtccggactggggctgccgccgccgtggcagccggcgctaccccgacttttc

SEQ ID NO: 44 - DNA, 489 bp, Mycobacterium tuberculosis
ggcgatctggtgagcccgggctgcgcggaatacgcggcagccaatcccactgggccggcctcggtgcagggaatgtcgcaggacccggtcgcggtggcggcctcgaacaatccggagttgacaacgctgacggctgcactgtcgggccagctcaatccgcaagtaaacctggtggacaccctcaacagcggtcagtacacggtgttcgcaccgaccaacgcggcatttagcaagctgccggcatccacgatcgacgagctcaagaccaattcgtcactgctgaccagcatcctgacctaccacgtagtggccggccaaaccagcccggccaacgtcgtcggcacccgtcagaccctccagggcgccagcgtgacggtgaccggtcagggtaacagcctcaaggtcggtaacgccgacgtcgtctgtggtggggtgtctaccgccaacgcgacggtgtacatgattgacagcgtgctaatgcctccggcg

SEQ ID NO: 45 - DNA, 282 bp, Mycobacterium tuberculosis
atgaccatcaactatcaattcggggacgtcgacgctcacggcgccatgatccgcgctcaggccgggtcgctggaggccgagcatcaggccatcatttctgatgtgttgaccgcgagtgacttttggggcggcgccggttcggcggcctgccaggggttcattacccagctgggccgtaacttccaggtgatctacgagcaggccaacgcccacgggcagaaggtgcaggctgccggcaacaacatggcacaaaccgacagcgccgtcggctccagctgggcc

SEQ ID NO: 46 - DNA, 294 bp, Mycobacterium tuberculosis
atgacctcgcgttttatgacggatccgcacgcgatgcgggacatggcgggccgttttgaggtgcacgcccagacggtggaggacgaggctcgccggatgtgggcgtccgcgcaaaacatttccggcgcgggctggagtggcatggccgaggcgacctcgctagacaccatgacccagatgaatcaggcgtttcgcaacatcgtgaacatgctgcacggggtgcgtgacgggctggttcgcgacgccaacaactacgaacagcaagagcaggcctcccagcagatcctcagcagc

SEQ ID NO: 47 - DNA, 786 bp, Mycobacterium tuberculosis
agtccttgtgcatattttcttgtctacgaatcaaccgaaacgaccgagcggcccgagcaccatgaattcaagcaggcggcggtgttgaccgacctgcccggcgagctgatgtccgcgctatcgcaggggttgtcccagttcgggatcaacataccgccggtgcccagcctgaccgggagcggcgatgccagcacgggtctaaccggtcctggcctgactagtccgggattgaccagcccgggattgaccagcccgggcctcaccgaccctgcccttaccagtccgggcctgacgccaaccctgcccggatcactcgccgcgcccggcaccaccctggcgccaacgcccggcgtgggggccaatccggcgctcaccaaccccgcgctgaccagcccgaccggggcgacgccgggattgaccagcccgacgggtttggatcccgcgctgggcggcgccaacgaaatcccgattacgacgccggtcggattggatcccggggctgacggcacctatccgatcctcggtgatccaacactggggaccataccgagcagccccgccaccacctccaccggcggcggcggtctcgtcaacgacgtgatgcaggtggccaacgagttgggcgccagtcaggctatcgacctgctaaaaggtgtgctaatgccgtcgatcatgcaggccgtccagaatggcggcgcggccgcgccggcagccagcccgccggtcccgcccatccccgcggccgcggcggtgccaccgacggacccaatcaccgtgccggtcgcc

SEQ ID NO: 48 - DNA, 1635 bp, Artificial Sequence
  Synthetic Construct
ggtacccatctcgccaacggttcgatgtcggaagtcatgatgtcggaaattgccgggttgcctatccctccgattatccattacggggcgattgcctatgcccccagcggcgcgtcgggcaaagcgtggcaccagcgcacaccggcgcgagcagagcaagtcgcactagaaaagtgcggtgacaagacttgcaaagtggttagtcgcttcaccaggtgcggcgcggtcgcctacaacggctcgaaataccaaggcggaaccggactcacgcgccgcgcggcagaagacgacgccgtgaaccgactcgaaggcgggcggatcgtcaactgggcgtgcaacgagctcatgacctcgcgttttatgacggatccgcacgcgatgcgggacatggcgggccgttttgaggtgcacgcccagacggtggaggacgaggctcgccggatgtgggcgtccgcgcaaaacatctcgggcgcgggctggagtggcatggccgaggcgacctcgctagacaccatgacccagatgaatcaggcgtttcgcaacatcgtgaacatgctgcacggggtgcgtgacgggctggttcgcgacgccaacaactacgaacagcaagagcaggcctcccagcagatcctcagcagcgtcgacgtggtcgatgcccaccgcggcggccacccgaccccgatgagctcgacgaaggccacgctgcggctggccgaggccaccgacagctcgggcaagatcaccaagcgcggagccgacaagctgatttccaccatcgacgaattcgccaagattgccatcagctcgggctgtgccgagctgatggccttcgccacgtcggcggtccgcgacgccgagaattccgaggacgtcctgtcccgggtgcgcaaagagaccggtgtcgagttgcaggcgctgcgtggggaggacgagtcacggctgaccttcctggccgtgcgacgatggtacgggtggagcgctgggcgcatcctcaacctcgacatcggcggcggctcgctggaagtgtccagtggcgtggacgaggagcccgagattgcgttatcgctgcccctgggcgccggacggttgacccgagagtggctgcccgacgatccgccgggccggcgccgggtggcgatgctgcgagactggctggatgccgagctggccgagcccagtgtgaccgtcctggaagccggcagccccgacctggcggtcgcaacgtcgaagacgtttcgctcgttggcgcgactaaccggtgcggccccatccatggccgggccgcgggtgaagaggaccctaacggcaaatggtctgcggcaactcatcgcgtttatctctaggatgacggcggttgaccgtgcagaactggaaggggtaagcgccgaccgagcgccgcagattgtggccggcgccctggtggcagaggcgagcatgcgagcactgtcgatagaagcggtggaaatctgcccgtgggcgctgcgggaaggtctcatcttgcgcaaactcgacagcgaagccgacggaaccgccctcatcgagtcttcgtctgtgcacacttcggtgcgtgccgtcggaggtcagccagctgatcggaacgcggccaaccgatcgagaggcagcaaaccaagtact

SEQ ID NO: 49 - DNA, 1950 bp, Artificial Sequence
  Synthetic Construct
gacgacatcgattgggacgccatcgcgcaatgcgaatccggcggcaattgggcggccaacaccggtaacgggttatacggtggtctgcagatcagccaggcgacgtgggattccaacggtggtgtcgggtcgccggcggccgcgagtccccagcaacagatcgaggtcgcagacaacattatgaaaacccaaggcccgggtgcgtggccgaaatgtagttcttgtagtcagggagacgcaccgctgggctcgctcacccacatcctgacgttcctcgcggccgagactggaggttgttcggggagcagggacgatggtacccatctcgccaacggttcgatgtcggaagtcatgatgtcggaaattgccgggttgcctatccctccgattatccattacggggcgattgcctatgcccccagcggcgcgtcgggcaaagcgtggcaccagcgcacaccggcgcgagcagagcaagtcgcactagaaaagtgcggtgacaagacttgcaaagtggttagtcgcttcaccaggtgcggcgcggtcgcctacaacggctcgaaataccaaggcggaaccggactcacgcgccgcgcggcagaagacgacgccgtgaaccgactcgaaggcgggcggatcgtcaactgggcgtgcaacgagctcatgacctcgcgttttatgacggatccgcacgcgatgcgggacatggcgggccgttttgaggtgcacgcccagacggtggaggacgaggctcgccggatgtgggcgtccgcgcaaaacatctcgggcgcgggctggagtggcatggccgaggcgacctcgctagacaccatgacccagatgaatcaggcgtttcgcaacatcgtgaacatgctgcacggggtgcgtgacgggctggttcgcgacgccaacaactacgaacagcaagagcaggcctcccagcagatcctcagcagcgtcgacatggtcgatgcccaccgcggcggccacccgaccccgatgagctcgacgaaggccacgctgcggctggccgaggccaccgacagctcgggcaagatcaccaagcgcggagccgacaagctgatttccaccatcgacgaattcgccaagattgccatcagctcgggctgtgccgagctgatggccttcgccacgtcggcggtccgcgacgccgagaattccgaggacgtcctgtcccgggtgcgcaaagagaccggtgtcgagttgcaggcgctgcgtggggaggacgagtcacggctgaccttcctggccgtgcgacgatggtacgggtggagcgctgggcgcatcctcaacctcgacatcggcggcggctcgctggaagtgtccagtggcgtggacgaggagcccgagattgcgttatcgctgcccctgggcgccggacggttgacccgagagtggctgcccgacgatccgccgggccggcgccgggtggcgatgctgcgagactggctggatgccgagctggccgagcccagtgtgaccgtcctggaagccggcagccccgacctggcggtcgcaacgtcgaagacgtttcgctcgttggcgcgactaaccggtgcggccccatccatggccgggccgcgggtgaagaggaccctaacggcaaatggtctgcggcaactcatcgcgtttatctctaggatgacggcggttgaccgtgcagaactggaaggggtaagcgccgaccgagcgccgcagattgtggccggcgccctggtggcagaggcgagcatgcgagcactgtcgatagaagcggtggaaatctgcccgtgggcgctgcgggaaggtctcatcttgcgcaaactcgacagcgaagccgacggaaccgccctcatcgagtcttcgtctgtgcacacttcggtgcgtgccgtcggaggtcagccagctgatcggaacgcggccaaccgatcgagaggcagcaaaccaagtact

SEQ ID NO: 50 - DNA, 2016 bp, Artificial Sequence
  Synthetic Construct
catatgatgaccatcaactatcaattcggggacgtcgacgctcacggcgccatgatccgcgctcaggccgggtcgctggaggccgagcatcaggccatcatttctgatgtgttgaccgcgagtgacttttggggcggcgccggttcggcggcctgccaggggttcattacccagctgggccgtaacttccaggtgatctacgagcaggccaacgcccacgggcagaaggtgcaggctgccggcaacaacatggcacaaaccgacagcgccgtcggctccagctgggccggtaccgacgacatcgattgggacgccatcgcgcaatgcgaatccggcggcaattgggcggccaacaccggtaacgggttatacggtggtctgcagatcagccaggcgacgtgggattccaacggtggtgtcgggtcgccggcggccgcgagtccccagcaacagatcgaggtcgcagacaacattatgaaaacccaaggcccgggtgcgtggccgaaatgtagttcttgtagtcagggagacgcaccgctgggctcgctcacccacatcctgacgttcctcgcggccgagactggaggttgttcggggagcagggacgatggatccgtggtggatttcggggcgttaccaccggagatcaactccgcgaggatgtacgccggcccgggttcggcctcgctggtggccgccgcgaagatgtgggacagcgtggcgagtgacctgttttcggccgcgtcggcgtttcagtcggtggtctggggtctgacggtggggtcgtggataggttcgtcggcgggtctgatggcggcggcggcctcgccgtatgtggcgtggatgagcgtcaccgcggggcaggcccagctgaccgccgcccaggtccgggttgctgcggcggcctacgagacagcgtataggctgacggtgcccccgccggtgatcgccgagaaccgtaccgaactgatgacgctgaccgcgaccaacctcttggggcaaaacacgccggcgatcgaggccaatcaggccgcatacagccagatgtggggccaagacgcggaggcgatgtatggctacgccgccacggcggcgacggcgaccgaggcgttgctgccgttcgaggacgccccactgatcaccaaccccggcggggaattcttctcccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccttcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatggggcctagcctgatcggcctcgcgatgggtgacgccggcggttacaaggccgcagacatgtggggtccctcgagtgacccggcatgggagcgcaacgaccctacgcagcagatccccaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaacgccgcgggcgggcacaacgccgtgttcaacttcccgcccaacggcacgcacagctgggagtactggggcgctcagctcaacgccatgaagggtgacctgcagagttcgttaggcgccggc

SEQ ID NO: 51 - DNA, 2392 bp, Artificial Sequence
  Synthetic Construct
  misc_feature 648: n = A,T,C or G
ggtacccatctcgccaacggttcgatgtcggaagtcatgatgtcggaaattgccgggttgcctatccctccgattatccattacggggcgattgcctatgcccccagcggcgcgtcgggcaaagcgtggcaccagcgcacaccggcgcgagcagagcaagtcgcactagaaaagtgcggtgacaagacttgcaaagtggttagtcgcttcaccaggtgcggcgcggtcgcctacaacggctcgaaataccaaggcggaaccggactcacgcgccgcgcggcagaagacgacgccgtgaaccgactcgaaggcgggcggatcgtcaactgggcgtgcaacgagctcatgacctcgcgttttatgacggatccgcacgcgatgcgggacatggcgggccgttttgaggtgcacgcccagacggtggaggacgaggctcgccggatgtgggcgtccgcgcaaaacatctcgggcgcgggctggagtggcatggccgaggcgacctcgctagacaccatgacccagatgaatcaggcgtttcgcaacatcgtgaacatgctgcacggggtgcgtgacgggctggttcgcgacgccaacaactacgaacagcaagagcaggcctcccagcagatcctcagcagcgtcgacatncaatttcgccgttttgccgccggaggtgaattcggcgcgcatattcgccggtgcgggcctgggcccaatgctggcggcggcgtcggcctgggacgggttggccgaggagttgcatgccgcggcgggctcgttcgcgtcggtgaccaccgggttggcgggcgacgcgtggcatggtccggcgtcgctggcgatgacccgcgcggccagcccgtatgtggggtggttgaacacggcggcgggtcaggccgcgcaggcggccggccaggcgcggctagcggcgagcgcgttcgaggcgacgctggcggccaccgtgtctccagcgatggtcgcggccaaccggacacggctggcgtcgctggtggcagccaacttgctgggccagaacgccccggcgatcgcggccgcggaggctgaatacgagcagatatgggcccaggacgtggccgcgatgttcggctatcactccgccgcgtcggcggtggccacgcagctggcgcctattcaagagggtttgcagcagcagctgcaaaacgtgctggcccagttggctagcgggaacctgggcagcggaaatgtgggcgtcggcaacatcggcaacgacaacattggcaacgcaaacatcggcttcggaaatcgaggcgacgccaacatcggcatcgggaatatcggcgacagaaacctcggcattgggaacaccggcaattggaatatcggcatcggcatcaccggcaacggacaaatcggcttcggcaagcctgccaaccccgacgtcttggtggtgggcaacggcggcccgggagtaaccgcgttggtcatgggcggcaccgacagcctactgccgctgcccaacatccccttactcgagtacgctgcgcggttcatcacccccgtgcatcccggatacaccgctacgttcctggaaacgccatcgcagtttttcccattcaccgggctgaatagcctgacctatgacgtctccgtggcccagggcgtaacgaatctgcacaccgcgatcatggcgcaactcgcggcgggaaacgaagtcgtcgtcttcggcacctcccaaagcgccacgatagccaccttcgaaatgcgctatctgcaatccctgccagcacacctgcgtccgggtctcgacgaattgtcctttacgttgaccggcaatcccaaccggcccgacggtggcattcttacgcgttttggcttctccataccgcagttgggtttcacattgtccggcgcgacgcccgccgacgcctaccccaccgtcgattacgcgttccagtacgacggcgtcaacgacttccccaaatacccgctgaatgtcttcgcgaccgccaacgcgatcgcgggcatccttttcctgcactccgggttgattgcgttgccgcccgatcttgcctcgggcgtggttcaaccggtgtcctcaccggacgtcctgaccacctacatcctgctgcccagccaagatctgccgctgctggtcccgctgcgtgctatccccctgctgggaaacccgcttgccgacctcatccagccggacttgcgggtgctcgtcgagttgggttatgaccgcaccgcccaccaggacgtgcccagcccgttcggactgtttccggacgtcgattgggccgaggtggccgcggacctgcagcaaggcgccgtgcaaggcgtcaacgacgccctgtccggactggggctgccgccgccgtggcagccggcgctaccccgacttttcagtact

SEQ ID NO: 52 - DNA, 2538 bp, Artificial Sequence
  Synthetic Construct
atgggcgatctggtgagcccgggctgcgcggaatacgcggcagccaatcccactgggccggcctcggtgcagggaatgtcgcaggacccggtcgcggtggcggcctcgaacaatccggagttgacaacgctgacggctgcactgtcgggccagctcaatccgcaagtaaacctggtggacaccctcaacagcggtcagtacacggtgttcgcaccgaccaacgcggcatttagcaagctgccggcatccacgatcgacgagctcaagaccaattcgtcactgctgaccagcatcctgacctaccacgtagtggccggccaaaccagcccggccaacgtcgtcggcacccgtcagaccctccagggcgccagcgtgacggtgaccggtcagggtaacagcctcaaggtcggtaacgccgacgtcgtctgtggtggggtgtctaccgccaacgcgacggtgtacatgattgacagcgtgctaatgcctccggcgggatccgtggtggatttcggggcgttaccaccggagatcaactccgcgaggatgtacgccggcccgggttcggcctcgctggtggccgccgcgaagatgtgggacagcgtggcgagtgacctgttttcggccgcgtcggcgtttcagtcggtggtctggggtctgacggtggggtcgtggataggttcgtcggcgggtctgatggcggcggcggcctcgccgtatgtggcgtggatgagcgtcaccgcggggcaggcccagctgaccgccgcccaggtccgggttgctgcggcggcctacgagacagcgtataggctgacggtgcccccgccggtgatcgccgagaaccgtaccgaactgatgacgctgaccgcgaccaacctcttggggcaaaacacgccggcgatcgaggccaatcaggccgcatacagccagatgtggggccaagacgcggaggcgatgtatggctacgccgccacggcggcgacggcgaccgaggcgttgctgccgttcgaggacgccccactgatcaccaaccccggcgggctccttgagcaggccgtcgcggtcgaggaggccatcgacaccgccgcggcgaaccagttgatgaacaatgtgccccaagcgctgcaacagctggcccagccagcgcagggcgtcgtaccttcttccaagctgggtgggctgtggacggcggtctcgccgcatctgtcgccgctcagcaacgtcagttcgatagccaacaaccacatgtcgatgatgggcacgggtgtgtcgatgaccaacaccttgcactcgatgttgaagggcttagctccggcggcggctcaggccgtggaaaccgcggcggaaaacggggtctgggcgatgagctcgctgggcagccagctgggttcgtcgctgggttcttcgggtctgggcgctggggtggccgccaacttgggtcgggcggcctcggtcggttcgttgtcggtgccgccagcatgggccgcggccaaccaggcggtcaccccggcggcgcgggcgctgccgctgaccagcctgaccagcgccgcccaaaccgcccccggacacatgctgggcgggctaccgctggggcactcggtcaacgccggcagcggtatcaacaatgcgctgcgggtgccggcacgggcctacgcgataccccgcacaccggccgccggagaattcttctcccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccttcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatggggcctagcctgatcggcctcgcgatgggtgacgccggcggttacaaggccgcagacatgtggggtccctcgagtgacccggcatgggagcgcaacgaccctacgcagcagatccccaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaacgccgcgggcgggcacaacgccgtgttcaacttcccgcccaacggcacgcacagctgggagtactggggcgctcagctcaacgccatgaagggtgacctgcagagttcgttaggcgccggc

SEQ ID NO: 53 - DNA, 2649 bp, Artificial Sequence
  Synthetic Construct
atgaccatcaactatcaattcggggacgtcgacgctcacggcgccatgatccgcgctcaggccgggtcgctggaggccgagcatcaggccatcatttctgatgtgttgaccgcgagtgacttttggggcggcgccggttcggcggcctgccaggggttcattacccagctgggccgtaacttccaggtgatctacgagcaggccaacgcccacgggcagaaggtgcaggctgccggcaacaacatggcacaaaccgacagcgccgtcggctccagctgggccggtaccgacgacatcgattgggacgccatcgcgcaatgcgaatccggcggcaattgggcggccaacaccggtaacgggttatacggtggtctgcagatcagccaggcgacgtgggattccaacggtggtgtcgggtcgccggcggccgcgagtccccagcaacagatcgaggtcgcagacaacattatgaaaacccaaggcccgggtgcgtggccgaaatgtagttcttgtagtcagggagacgcaccgctgggctcgctcacccacatcctgacgttcctcgcggccgagactggaggttgttcggggagcagggacgatggatccgtggtggatttcggggcgttaccaccggagatcaactccgcgaggatgtacgccggcccgggttcggcctcgctggtggccgccgcgaagatgtgggacagcgtggcgagtgacctgttttcggccgcgtcggcgtttcagtcggtggtctggggtctgacggtggggtcgtggataggttcgtcggcgggtctgatggcggcggcggcctcgccgtatgtggcgtggatgagcgtcaccgcggggcaggcccagctgaccgccgcccaggtccgggttgctgcggcggcctacgagacagcgtataggctgacggtgcccccgccggtgatcgccgagaaccgtaccgaactgatgacgctgaccgcgaccaacctcttggggcaaaacacgccggcgatcgaggccaatcaggccgcatacagccagatgtggggccaagacgcggaggcgatgtatggctacgccgccacggcggcgacggcgaccgaggcgttgctgccgttcgaggacgccccactgatcaccaaccccggcgggctccttgagcaggccgtcgcggtcgaggaggccatcgacaccgccgcggcgaaccagttgatgaacaatgtgccccaagcgctgcaacagctggcccagccagcgcagggcgtcgtaccttcttccaagctgggtgggctgtggacggcggtctcgccgcatctgtcgccgctcagcaacgtcagttcgatagccaacaaccacatgtcgatgatgggcacgggtgtgtcgatgaccaacaccttgcactcgatgttgaagggcttagctccggcggcggctcaggccgtggaaaccgcggcggaaaacggggtctgggcgatgagctcgctgggcagccagctgggttcgtcgctgggttcttcgggtctgggcgctggggtggccgccaacttgggtcgggcggcctcggtcggttcgttgtcggtgccgccagcatgggccgcggccaaccaggcggtcaccccggcggcgcgggcgctgccgctgaccagcctgaccagcgccgcccaaaccgcccccggacacatgctgggcgggctaccgctggggcactcggtcaacgccggcagcggtatcaacaatgcgctgcgggtgccggcacgggcctacgcgataccccgcacaccggccgccggagaattcttctcccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccttcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatggggcctagcctgatcggcctcgcgatgggtgacgccggcggttacaaggccgcagacatgtggggtccctcgagtgacccggcatgggagcgcaacgaccctacgcagcagatccccaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaacgccgcgggcgggcacaacgccgtgttcaacttcccgcccaacggcacgcacagctgggagtactggggcgctcagctcaacgccatgaagggtgacctgcagagttcgttaggcgccggc

SEQ ID NO: 54 - DNA, 2673 bp, Artificial Sequence
  Synthetic Construct
  misc_feature 930: n = A,T,C or G
atgaccatcaactatcaattcggggacgtcgacgctcacggcgccatgatccgcgctcaggccgggtcgctggaggccgagcatcaggccatcatttctgatgtgttgaccgcgagtgacttttggggcggcgccggttcggcggcctgccaggggttcattacccagctgggccgtaacttccaggtgatctacgagcaggccaacgcccacgggcagaaggtgcaggctgccggcaacaacatggcacaaaccgacagcgccgtcggctccagctgggccggtacccatctcgccaacggttcgatgtcggaagtcatgatgtcggaaattgccgggttgcctatccctccgattatccattacggggcgattgcctatgcccccagcggcgcgtcgggcaaagcgtggcaccagcgcacaccggcgcgagcagagcaagtcgcactagaaaagtgcggtgacaagacttgcaaagtggttagtcgcttcaccaggtgcggcgcggtcgcctacaacggctcgaaataccaaggcggaaccggactcacgcgccgcgcggcagaagacgacgccgtgaaccgactcgaaggcgggcggatcgtcaactgggcgtgcaacgagctcatgacctcgcgttttatgacggatccgcacgcgatgcgggacatggcgggccgttttgaggtgcacgcccagacggtggaggacgaggctcgccggatgtgggcgtccgcgcaaaacatctcgggcgcgggctggagtggcatggccgaggcgacctcgctagacaccatgacccagatgaatcaggcgtttcgcaacatcgtgaacatgctgcacggggtgcgtgacgggctggttcgcgacgccaacaactacgaacagcaagagcaggcctcccagcagatcctcagcagcgtcgacatnaatttcgccgttttgccgccggaggtgaattcggcgcgcatattcgccggtgcgggcctgggcccaatgctggcggcggcgtcggcctgggacgggttggccgaggagttgcatgccgcggcgggctcgttcgcgtcggtgaccaccgggttggcgggcgacgcgtggcatggtccggcgtcgctggcgatgacccgcgcggccagcccgtatgtggggtggttgaacacggcggcgggtcaggccgcgcaggcggccggccaggcgcggctagcggcgagcgcgttcgaggcgacgctggcggccaccgtgtctccagcgatggtcgcggccaaccggacacggctggcgtcgctggtggcagccaacttgctgggccagaacgccccggcgatcgcggccgcggaggctgaatacgagcagatatgggcccaggacgtggccgcgatgttcggctatcactccgccgcgtcggcggtggccacgcagctggcgcctattcaagagggtttgcagcagcagctgcaaaacgtgctggcccagttggctagcgggaacctgggcagcggaaatgtgggcgtcggcaacatcggcaacgacaacattggcaacgcaaacatcggcttcggaaatcgaggcgacgccaacatcggcatcgggaatatcggcgacagaaacctcggcattgggaacaccggcaattggaatatcggcatcggcatcaccggcaacggacaaatcggcttcggcaagcctgccaaccccgacgtcttggtggtgggcaacggcggcccgggagtaaccgcgttggtcatgggcggcaccgacagcctactgccgctgcccaacatccccttactcgagtacgctgcgcggttcatcacccccgtgcatcccggatacaccgctacgttcctggaaacgccatcgcagtttttcccattcaccgggctgaatagcctgacctatgacgtctccgtggcccagggcgtaacgaatctgcacaccgcgatcatggcgcaactcgcggcgggaaacgaagtcgtcgtcttcggcacctcccaaagcgccacgatagccaccttcgaaatgcgctatctgcaatccctgccagcacacctgcgtccgggtctcgacgaattgtcctttacgttgaccggcaatcccaaccggcccgacggtggcattcttacgcgttttggcttctccataccgcagttgggtttcacattgtccggcgcgacgcccgccgacgcctaccccaccgtcgattacgcgttccagtacgacggcgtcaacgacttccccaaatacccgctgaatgtcttcgcgaccgccaacgcgatcgcgggcatccttttcctgcactccgggttgattgcgttgccgcccgatcttgcctcgggcgtggttcaaccggtgtcctcaccggacgtcctgaccacctacatcctgctgcccagccaagatctgccgctgctggtcccgctgcgtgctatccccctgctgggaaacccgcttgccgacctcatccagccggacttgcgggtgctcgtcgagttgggttatgaccgcaccgcccaccaggacgtgcccagcccgttcggactgtttccggacgtcgattgggccgaggtggccgcggacctgcagcaaggcgccgtgcaaggcgtcaacgacgccctgtccggactggggctgccgccgccgtggcagccggcgctaccccgacttttcagtact

SEQ ID NO: 55 - DNA, 2707 bp, Artificial Sequence
  Synthetic Construct
  misc_feature 964: n = A,T,C or G
ggacgacatcgattgggacgccatcgcgcaatgcgaatccggcggcaattgggcggccaacaccggtaacgggttatacggtggtctgcagatcagccaggcgacgtgggattccaacggtggtgtcgggtcgccggcggccgcgagtccccagcaacagatcgaggtcgcagacaacattatgaaaacccaaggcccgggtgcgtggccgaaatgtagttcttgtagtcagggagacgcaccgctgggctcgctcacccacatcctgacgttcctcgcggccgagactggaggttgttcggggagcagggacgatggtacccatctcgccaacggttcgatgtcggaagtcatgatgtcggaaattgccgggttgcctatccctccgattatccattacggggcgattgcctatgcccccagcggcgcgtcgggcaaagcgtggcaccagcgcacaccggcgcgagcagagcaagtcgcactagaaaagtgcggtgacaagacttgcaaagtggttagtcgcttcaccaggtgcggcgcggtcgcctacaacggctcgaaataccaaggcggaaccggactcacgcgccgcgcggcagaagacgacgccgtgaaccgactcgaaggcgggcggatcgtcaactgggcgtgcaacgagctcatgacctcgcgttttatgacggatccgcacgcgatgcgggacatggcgggccgttttgaggtgcacgcccagacggtggaggacgaggctcgccggatgtgggcgtccgcgcaaaacatctcgggcgcgggctggagtggcatggccgaggcgacctcgctagacaccatgacccagatgaatcaggcgtttcgcaacatcgtgaacatgctgcacggggtgcgtgacgggctggttcgcgacgccaacaactacgaacagcaagagcaggcctcccagcagatcctcagcagcgtcgacatnaatttcgccgttttgccgccggaggtgaattcggcgcgcatattcgccggtgcgggcctgggcccaatgctggcggcggcgtcggcctgggacgggttggccgaggagttgcatgccgcggcgggctcgttcgcgtcggtgaccaccgggttggcgggcgacgcgtggcatggtccggcgtcgctggcgatgacccgcgcggccagcccgtatgtggggtggttgaacacggcggcgggtcaggccgcgcaggcggccggccaggcgcggctagcggcgagcgcgttcgaggcgacgctggcggccaccgtgtctccagcgatggtcgcggccaaccggacacggctggcgtcgctggtggcagccaacttgctgggccagaacgccccggcgatcgcggccgcggaggctgaatacgagcagatatgggcccaggacgtggccgcgatgttcggctatcactccgccgcgtcggcggtggccacgcagctggcgcctattcaagagggtttgcagcagcagctgcaaaacgtgctggcccagttggctagcgggaacctgggcagcggaaatgtgggcgtcggcaacatcggcaacgacaacattggcaacgcaaacatcggcttcggaaatcgaggcgacgccaacatcggcatcgggaatatcggcgacagaaacctcggcattgggaacaccggcaattggaatatcggcatcggcatcaccggcaacggacaaatcggcttcggcaagcctgccaaccccgacgtcttggtggtgggcaacggcggcccgggagtaaccgcgttggtcatgggcggcaccgacagcctactgccgctgcccaacatccccttactcgagtacgctgcgcggttcatcacccccgtgcatcccggatacaccgctacgttcctggaaacgccatcgcagtttttcccattcaccgggctgaatagcctgacctatgacgtctccgtggcccagggcgtaacgaatctgcacaccgcgatcatggcgcaactcgcggcgggaaacgaagtcgtcgtcttcggcacctcccaaagcgccacgatagccaccttcgaaatgcgctatctgcaatccctgccagcacacctgcgtccgggtctcgacgaattgtcctttacgttgaccggcaatcccaaccggcccgacggtggcattcttacgcgttttggcttctccataccgcagttgggtttcacattgtccggcgcgacgcccgccgacgcctaccccaccgtcgattacgcgttccagtacgacggcgtcaacgacttccccaaatacccgctgaatgtcttcgcgaccgccaacgcgatcgcgggcatccttttcctgcactccgggttgattgcgttgccgcccgatcttgcctcgggcgtggttcaaccggtgtcctcaccggacgtcctgaccacctacatcctgctgcccagccaagatctgccgctgctggtcccgctgcgtgctatccccctgctgggaaacccgcttgccgacctcatccagccggacttgcgggtgctcgtcgagttgggttatgaccgcaccgcccaccaggacgtgcccagcccgttcggactgtttccggacgtcgattgggccgaggtggccgcggacctgcagcaaggcgccgtgcaaggcgtcaacgacgccctgtccggactggggctgccgccgccgtggcagccggcgctaccccgacttttcagtact

SEQ ID NO: 56 - DNA, 2742 bp, Artificial Sequence
  Synthetic Construct
gacgacatcgattgggacgccatcgcgcaatgcgaatccggcggcaattgggcggccaacaccggtaacgggttatacggtggtctgcagatcagccaggcgacgtgggattccaacggtggtgtcgggtcgccggcggccgcgagtccccagcaacagatcgaggtcgcagacaacattatgaaaacccaaggcccgggtgcgtggccgaaatgtagttcttgtagtcagggagacgcaccgctgggctcgctcacccacatcctgacgttcctcgcggccgagactggaggttgttcggggagcagggacgatgagctcagtccttgtgcatattttcttgtctacgaatcaaccgaaacgaccgagcggcccgagcaccatgaattcaagcaggcggcggtgttgaccgacctgcccggcgagctgatgtccgcgctatcgcaggggttgtcccagttcgggatcaacataccgccggtgcccagcctgaccgggagcggcgatgccagcacgggtctaaccggtcctggcctgactagtccgggattgaccagcccgggattgaccagcccgggcctcaccgaccctgcccttaccagtccgggcctgacgccaaccctgcccggatcactcgccgcgcccggcaccaccctggcgccaacgcccggcgtgggggccaatccggcgctcaccaaccccgcgctgaccagcccgaccggggcgacgccgggattgaccagcccgacgggtttggatcccgcgctgggcggcgccaacgaaatcccgattacgacgccggtcggattggatcccggggctgacggcacctatccgatcctcggtgatccaacactggggaccataccgagcagccccgccaccacctccaccggcggcggcggtctcgtcaacgacgtgatgcaggtggccaacgagttgggcgccagtcaggctatcgacctgctaaaaggtgtgctaatgccgtcgatcatgcaggccgtccagaatggcggcgcggccgcgccggcagccagcccgccggtcccgcccatccccgcggccgcggcggtgccaccgacggacccaatcaccgtgccggtcgccggtacccatctcgccaacggttcgatgtcggaagtcatgatgtcggaaattgccgggttgcctatccctccgattatccattacggggcgattgcctatgcccccagcggcgcgtcgggcaaagcgtggcaccagcgcacaccggcgcgagcagagcaagtcgcactagaaaagtgcggtgacaagacttgcaaagtggttagtcgcttcaccaggtgcggcgcggtcgcctacaacggctcgaaataccaaggcggaaccggactcacgcgccgcgcggcagaagacgacgccgtgaaccgactcgaaggcgggcggatcgtcaactgggcgtgcaacgagctcatgacctcgcgttttatgacggatccgcacgcgatgcgggacatggcgggccgttttgaggtgcacgcccagacggtggaggacgaggctcgccggatgtgggcgtccgcgcaaaacatctcgggcgcgggctggagtggcatggccgaggcgacctcgctagacaccatgacccagatgaatcaggcgtttcgcaacatcgtgaacatgctgcacggggtgcgtgacgggctggttcgcgacgccaacaactacgaacagcaagagcaggcctcccagcagatcctcagcagcgtcgacatggtcgatgcccaccgcggcggccacccgaccccgatgagctcgacgaaggccacgctgcggctggccgaggccaccgacagctcgggcaagatcaccaagcgcggagccgacaagctgatttccaccatcgacgaattcgccaagattgccatcagctcgggctgtgccgagctgatggccttcgccacgtcggcggtccgcgacgccgagaattccgaggacgtcctgtcccgggtgcgcaaagagaccggtgtcgagttgcaggcgctgcgtggggaggacgagtcacggctgaccttcctggccgtgcgacgatggtacgggtggagcgctgggcgcatcctcaacctcgacatcggcggcggctcgctggaagtgtccagtggcgtggacgaggagcccgagattgcgttatcgctgcccctgggcgccggacggttgacccgagagtggctgcccgacgatccgccgggccggcgccgggtggcgatgctgcgagactggctggatgccgagctggccgagcccagtgtgaccgtcctggaagccggcagccccgacctggcggtcgcaacgtcgaagacgtttcgctcgttggcgcgactaaccggtgcggccccatccatggccgggccgcgggtgaagaggaccctaacggcaaatggtctgcggcaactcatcgcgtttatctctaggatgacggcggttgaccgtgcagaactggaaggggtaagcgccgaccgagcgccgcagattgtggccggcgccctggtggcagaggcgagcatgcgagcactgtcgatagaagcggtggaaatctgcccgtgggcgctgcgggaaggtctcatcttgcgcaaactcgacagcgaagccgacggaaccgccctcatcgagtcttcgtctgtgcacacttcggtgcgtgccgtcggaggtcagccagctgatcggaacgcggccaaccgatcgagaggcagcaaaccaagtact

SEQ ID NO: 57 - DNA, 2826 bp, Artificial Sequence
  Synthetic Construct
atgaccatcaactatcaattcggggacgtcgacgctcacggcgccatgatccgcgctcaggccgggtcgctggaggccgagcatcaggccatcatttctgatgtgttgaccgcgagtgacttttggggcggcgccggttcggcggcctgccaggggttcattacccagctgggccgtaacttccaggtgatctacgagcaggccaacgcccacgggcagaaggtgcaggctgccggcaacaacatggcacaaaccgacagcgccgtcggctccagctgggccggtaccatgggcgatctggtgagcccgggctgcgcggaatacgcggcagccaatcccactgggccggcctcggtgcagggaatgtcgcaggacccggtcgcggtggcggcctcgaacaatccggagttgacaacgctgacggctgcactgtcgggccagctcaatccgcaagtaaacctggtggacaccctcaacagcggtcagtacacggtgttcgcaccgaccaacgcggcatttagcaagctgccggcatccacgatcgacgagctcaagaccaattcgtcactgctgaccagcatcctgacctaccacgtagtggccggccaaaccagcccggccaacgtcgtcggcacccgtcagaccctccagggcgccagcgtgacggtgaccggtcagggtaacagcctcaaggtcggtaacgccgacgtcgtctgtggtggggtgtctaccgccaacgcgacggtgtacatgattgacagcgtgctaatgcctccggcgggatccgtggtggatttcggggcgttaccaccggagatcaactccgcgaggatgtacgccggcccgggttcggcctcgctggtggccgccgcgaagatgtgggacagcgtggcgagtgacctgttttcggccgcgtcggcgtttcagtcggtggtctggggtctgacggtggggtcgtggataggttcgtcggcgggtctgatggcggcggcggcctcgccgtatgtggcgtggatgagcgtcaccgcggggcaggcccagctgaccgccgcccaggtccgggttgctgcggcggcctacgagacagcgtataggctgacggtgcccccgccggtgatcgccgagaaccgtaccgaactgatgacgctgaccgcgaccaacctcttggggcaaaacacgccggcgatcgaggccaatcaggccgcatacagccagatgtggggccaagacgcggaggcgatgtatggctacgccgccacggcggcgacggcgaccgaggcgttgctgccgttcgaggacgccccactgatcaccaaccccggcgggctccttgagcaggccgtcgcggtcgaggaggccatcgacaccgccgcggcgaaccagttgatgaacaatgtgccccaagcgctgcaacagctggcccagccagcgcagggcgtcgtaccttcttccaagctgggtgggctgtggacggcggtctcgccgcatctgtcgccgctcagcaacgtcagttcgatagccaacaaccacatgtcgatgatgggcacgggtgtgtcgatgaccaacaccttgcactcgatgttgaagggcttagctccggcggcggctcaggccgtggaaaccgcggcggaaaacggggtctgggcgatgagctcgctgggcagccagctgggttcgtcgctgggttcttcgggtctgggcgctggggtggccgccaacttgggtcgggcggcctcggtcggttcgttgtcggtgccgccagcatgggccgcggccaaccaggcggtcaccccggcggcgcgggcgctgccgctgaccagcctgaccagcgccgcccaaaccgcccccggacacatgctgggcgggctaccgctggggcactcggtcaacgccggcagcggtatcaacaatgcgctgcgggtgccggcacgggcctacgcgataccccgcacaccggccgccggagaattcttctcccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccttcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatggggcctagcctgatcggcctcgcgatgggtgacgccggcggttacaaggccgcagacatgtggggtccctcgagtgacccggcatgggagcgcaacgaccctacgcagcagatccccaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaacgccgcgggcgggcacaacgccgtgttcaacttcccgcccaacggcacgcacagctgggagtactggggcgctcagctcaacgccatgaagggtgacctgcagagttcgttaggcgccggc

SEQ ID NO: 58 - DNA, 3246 bp, Artificial Sequence
  Synthetic Construct
ggtacccatctcgccaacggttcgatgtcggaagtcatgatgtcggaaattgccgggttgcctatccctccgattatccattacggggcgattgcctatgcccccagcggcgcgtcgggcaaagcgtggcaccagcgcacaccggcgcgagcagagcaagtcgcactagaaaagtgcggtgacaagacttgcaaagtggttagtcgcttcaccaggtgcggcgcggtcgcctacaacggctcgaaataccaaggcggaaccggactcacgcgccgcgcggcagaagacgacgccgtgaaccgactcgaaggcgggcggatcgtcaactgggcgtgcaacgagctcatgacctcgcgttttatgacggatccgcacgcgatgcgggacatggcgggccgttttgaggtgcacgcccagacggtggaggacgaggctcgccggatgtgggcgtccgcgcaaaacatctcgggcgcgggctggagtggcatggccgaggcgacctcgctagacaccatgacccagatgaatcaggcgtttcgcaacatcgtgaacatgctgcacggggtgcgtgacgggctggttcgcgacgccaacaactacgaacagcaagagcaggcctcccagcagatcctcagcagcgtcgacatcaatttcgccgttttgccgccggaggtgaattcggcgcgcatattcgccggtgcgggcctgggcccaatgctggcggcggcgtcggcctgggacgggttggccgaggagttgcatgccgcggcgggctcgttcgcgtcggtgaccaccgggttggcgggcgacgcgtggcatggtccggcgtcgctggcgatgacccgcgcggccagcccgtatgtggggtggttgaacacggcggcgggtcaggccgcgcaggcggccggccaggcgcggctagcggcgagcgcgttcgaggcgacgctggcggccaccgtgtctccagcgatggtcgcggccaaccggacacggctggcgtcgctggtggcagccaacttgctgggccagaacgccccggcgatcgcggccgcggaggctgaatacgagcagatatgggcccaggacgtggccgcgatgttcggctatcactccgccgcgtcggcggtggccacgcagctggcgcctattcaagagggtttgcagcagcagctgcaaaacgtgctggcccagttggctagcgggaacctgggcagcggaaatgtgggcgtcggcaacatcggcaacgacaacattggcaacgcaaacatcggcttcggaaatcgaggcgacgccaacatcggcatcgggaatatcggcgacagaaacctcggcattgggaacaccggcaattggaatatcggcatcggcatcaccggcaacggacaaatcggcttcggcaagcctgccaaccccgacgtcttggtggtgggcaacggcggcccgggagtaaccgcgttggtcatgggcggcaccgacagcctactgccgctgcccaacatccccttactcgagtacgctgcgcggttcatcacccccgtgcatcccggatacaccgctacgttcctggaaacgccatcgcagtttttcccattcaccgggctgaatagcctgacctatgacgtctccgtggcccagggcgtaacgaatctgcacaccgcgatcatggcgcaactcgcggcgggaaacgaagtcgtcgtcttcggcacctcccaaagcgccacgatagccaccttcgaaatgcgctatctgcaatccctgccagcacacctgcgtccgggtctcgacgaattgtcctttacgttgaccggcaatcccaaccggcccgacggtggcattcttacgcgttttggcttctccataccgcagttgggtttcacattgtccggcgcgacgcccgccgacgcctaccccaccgtcgattacgcgttccagtacgacggcgtcaacgacttccccaaatacccgctgaatgtcttcgcgaccgccaacgcgatcgcgggcatccttttcctgcactccgggttgattgcgttgccgcccgatcttgcctcgggcgtggttcaaccggtgtcctcaccggacgtcctgaccacctacatcctgctgcccagccaagatctgccgctgctggtcccgctgcgtgctatccccctgctgggaaacccgcttgccgacctcatccagccggacttgcgggtgctcgtcgagttgggttatgaccgcaccgcccaccaggacgtgcccagcccgttcggactgtttccggacgtcgattgggccgaggtggccgcggacctgcagcaaggcgccgtgcaaggcgtcaacgacgccctgtccggactggggctgccgccgccgtggcagccggcgctaccccgacttttcagtactttctcccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccttcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatggggcctagcctgatcggcctcgcgatgggtgacgccggcggttacaaggccgcagacatgtggggtccctcgagtgacccggcatgggagcgcaacgaccctacgcagcagatccccaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaacgccgcgggcgggcacaacgccgtgttcaacttcccgcccaacggcacgcacagctgggagtactggggcgctcagctcaacgccatgaagggtgacctgcagagttcgttaggcgccggc

SEQ ID NO: 59 - DNA, 3498 bp, Artificial Sequence
  Synthetic Construct
  misc_feature 1755: n = A,T,C or G
gacgacatcgattgggacgccatcgcgcaatgcgaatccggcggcaattgggcggccaacaccggtaacgggttatacggtggtctgcagatcagccaggcgacgtgggattccaacggtggtgtcgggtcgccggcggccgcgagtccccagcaacagatcgaggtcgcagacaacattatgaaaacccaaggcccgggtgcgtggccgaaatgtagttcttgtagtcagggagacgcaccgctgggctcgctcacccacatcctgacgttcctcgcggccgagactggaggttgttcggggagcagggacgatgagctcagtccttgtgcatattttcttgtctacgaatcaaccgaaacgaccgagcggcccgagcaccatgaattcaagcaggcggcggtgttgaccgacctgcccggcgagctgatgtccgcgctatcgcaggggttgtcccagttcgggatcaacataccgccggtgcccagcctgaccgggagcggcgatgccagcacgggtctaaccggtcctggcctgactagtccgggattgaccagcccgggattgaccagcccgggcctcaccgaccctgcccttaccagtccgggcctgacgccaaccctgcccggatcactcgccgcgcccggcaccaccctggcgccaacgcccggcgtgggggccaatccggcgctcaccaaccccgcgctgaccagcccgaccggggcgacgccgggattgaccagcccgacgggtttggatcccgcgctgggcggcgccaacgaaatcccgattacgacgccggtcggattggatcccggggctgacggcacctatccgatcctcggtgatccaacactggggaccataccgagcagccccgccaccacctccaccggcggcggcggtctcgtcaacgacgtgatgcaggtggccaacgagttgggcgccagtcaggctatcgacctgctaaaaggtgtgctaatgccgtcgatcatgcaggccgtccagaatggcggcgcggccgcgccggcagccagcccgccggtcccgcccatccccgcggccgcggcggtgccaccgacggacccaatcaccgtgccggtcgccggtacccatctcgccaacggttcgatgtcggaagtcatgatgtcggaaattgccgggttgcctatccctccgattatccattacggggcgattgcctatgcccccagcggcgcgtcgggcaaagcgtggcaccagcgcacaccggcgcgagcagagcaagtcgcactagaaaagtgcggtgacaagacttgcaaagtggttagtcgcttcaccaggtgcggcgcggtcgcctacaacggctcgaaataccaaggcggaaccggactcacgcgccgcgcggcagaagacgacgccgtgaaccgactcgaaggcgggcggatcgtcaactgggcgtgcaacgagctcatgacctcgcgttttatgacggatccgcacgcgatgcgggacatggcgggccgttttgaggtgcacgcccagacggtggaggacgaggctcgccggatgtgggcgtccgcgcaaaacatctcgggcgcgggctggagtggcatggccgaggcgacctcgctagacaccatgacccagatgaatcaggcgtttcgcaacatcgtgaacatgctgcacggggtgcgtgacgggctggttcgcgacgccaacaactacgaacagcaagagcaggcctcccagcagatcctcagcagcgtcgacatnaatttcgccgttttgccgccggaggtgaattcggcgcgcatattcgccggtgcgggcctgggcccaatgctggcggcggcgtcggcctgggacgggttggccgaggagttgcatgccgcggcgggctcgttcgcgtcggtgaccaccgggttggcgggcgacgcgtggcatggtccggcgtcgctggcgatgacccgcgcggccagcccgtatgtggggtggttgaacacggcggcgggtcaggccgcgcaggcggccggccaggcgcggctagcggcgagcgcgttcgaggcgacgctggcggccaccgtgtctccagcgatggtcgcggccaaccggacacggctggcgtcgctggtggcagccaacttgctgggccagaacgccccggcgatcgcggccgcggaggctgaatacgagcagatatgggcccaggacgtggccgcgatgttcggctatcactccgccgcgtcggcggtggccacgcagctggcgcctattcaagagggtttgcagcagcagctgcaaaacgtgctggcccagttggctagcgggaacctgggcagcggaaatgtgggcgtcggcaacatcggcaacgacaacattggcaacgcaaacatcggcttcggaaatcgaggcgacgccaacatcggcatcgggaatatcggcgacagaaacctcggcattgggaacaccggcaattggaatatcggcatcggcatcaccggcaacggacaaatcggcttcggcaagcctgccaaccccgacgtcttggtggtgggcaacggcggcccgggagtaaccgcgttggtcatgggcggcaccgacagcctactgccgctgcccaacatccccttactcgagtacgctgcgcggttcatcacccccgtgcatcccggatacaccgctacgttcctggaaacgccatcgcagtttttcccattcaccgggctgaatagcctgacctatgacgtctccgtggcccagggcgtaacgaatctgcacaccgcgatcatggcgcaactcgcggcgggaaacgaagtcgtcgtcttcggcacctcccaaagcgccacgatagccaccttcgaaatgcgctatctgcaatccctgccagcacacctgcgtccgggtctcgacgaattgtcctttacgttgaccggcaatcccaaccggcccgacggtggcattcttacgcgttttggcttctccataccgcagttgggtttcacattgtccggcgcgacgcccgccgacgcctaccccaccgtcgattacgcgttccagtacgacggcgtcaacgacttccccaaatacccgctgaatgtcttcgcgaccgccaacgcgatcgcgggcatccttttcctgcactccgggttgattgcgttgccgcccgatcttgcctcgggcgtggttcaaccggtgtcctcaccggacgtcctgaccacctacatcctgctgcccagccaagatctgccgctgctggtcccgctgcgtgctatccccctgctgggaaacccgcttgccgacctcatccagccggacttgcgggtgctcgtcgagttgggttatgaccgcaccgcccaccaggacgtgcccagcccgttcggactgtttccggacgtcgattgggccgaggtggccgcggacctgcagcaaggcgccgtgcaaggcgtcaacgacgccctgtccggactggggctgccgccgccgtggcagccggcgctaccccgacttttcagtact

SEQ ID NO: 60 - DNA, 3528 bp, Artificial Sequence
  Synthetic Construct
  misc_feature 930: n = A,T,C or G
atgaccatcaactatcaattcggggacgtcgacgctcacggcgccatgatccgcgctcaggccgggtcgctggaggccgagcatcaggccatcatttctgatgtgttgaccgcgagtgacttttggggcggcgccggttcggcggcctgccaggggttcattacccagctgggccgtaacttccaggtgatctacgagcaggccaacgcccacgggcagaaggtgcaggctgccggcaacaacatggcacaaaccgacagcgccgtcggctccagctgggccggtacccatctcgccaacggttcgatgtcggaagtcatgatgtcggaaattgccgggttgcctatccctccgattatccattacggggcgattgcctatgcccccagcggcgcgtcgggcaaagcgtggcaccagcgcacaccggcgcgagcagagcaagtcgcactagaaaagtgcggtgacaagacttgcaaagtggttagtcgcttcaccaggtgcggcgcggtcgcctacaacggctcgaaataccaaggcggaaccggactcacgcgccgcgcggcagaagacgacgccgtgaaccgactcgaaggcgggcggatcgtcaactgggcgtgcaacgagctcatgacctcgcgttttatgacggatccgcacgcgatgcgggacatggcgggccgttttgaggtgcacgcccagacggtggaggacgaggctcgccggatgtgggcgtccgcgcaaaacatctcgggcgcgggctggagtggcatggccgaggcgacctcgctagacaccatgacccagatgaatcaggcgtttcgcaacatcgtgaacatgctgcacggggtgcgtgacgggctggttcgcgacgccaacaactacgaacagcaagagcaggcctcccagcagatcctcagcagcgtcgacatnaatttcgccgttttgccgccggaggtgaattcggcgcgcatattcgccggtgcgggcctgggcccaatgctggcggcggcgtcggcctgggacgggttggccgaggagttgcatgccgcggcgggctcgttcgcgtcggtgaccaccgggttggcgggcgacgcgtggcatggtccggcgtcgctggcgatgacccgcgcggccagcccgtatgtggggtggttgaacacggcggcgggtcaggccgcgcaggcggccggccaggcgcggctagcggcgagcgcgttcgaggcgacgctggcggccaccgtgtctccagcgatggtcgcggccaaccggacacggctggcgtcgctggtggcagccaacttgctgggccagaacgccccggcgatcgcggccgcggaggctgaatacgagcagatatgggcccaggacgtggccgcgatgttcggctatcactccgccgcgtcggcggtggccacgcagctggcgcctattcaagagggtttgcagcagcagctgcaaaacgtgctggcccagttggctagcgggaacctgggcagcggaaatgtgggcgtcggcaacatcggcaacgacaacattggcaacgcaaacatcggcttcggaaatcgaggcgacgccaacatcggcatcgggaatatcggcgacagaaacctcggcattgggaacaccggcaattggaatatcggcatcggcatcaccggcaacggacaaatcggcttcggcaagcctgccaaccccgacgtcttggtggtgggcaacggcggcccgggagtaaccgcgttggtcatgggcggcaccgacagcctactgccgctgcccaacatccccttactcgagtacgctgcgcggttcatcacccccgtgcatcccggatacaccgctacgttcctggaaacgccatcgcagtttttcccattcaccgggctgaatagcctgacctatgacgtctccgtggcccagggcgtaacgaatctgcacaccgcgatcatggcgcaactcgcggcgggaaacgaagtcgtcgtcttcggcacctcccaaagcgccacgatagccaccttcgaaatgcgctatctgcaatccctgccagcacacctgcgtccgggtctcgacgaattgtcctttacgttgaccggcaatcccaaccggcccgacggtggcattcttacgcgttttggcttctccataccgcagttgggtttcacattgtccggcgcgacgcccgccgacgcctaccccaccgtcgattacgcgttccagtacgacggcgtcaacgacttccccaaatacccgctgaatgtcttcgcgaccgccaacgcgatcgcgggcatccttttcctgcactccgggttgattgcgttgccgcccgatcttgcctcgggcgtggttcaaccggtgtcctcaccggacgtcctgaccacctacatcctgctgcccagccaagatctgccgctgctggtcccgctgcgtgctatccccctgctgggaaacccgcttgccgacctcatccagccggacttgcgggtgctcgtcgagttgggttatgaccgcaccgcccaccaggacgtgcccagcccgttcggactgtttccggacgtcgattgggccgaggtggccgcggacctgcagcaaggcgccgtgcaaggcgtcaacgacgccctgtccggactggggctgccgccgccgtggcagccggcgctaccccgacttttcagtactttctcccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccttcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatggggcctagcctgatcggcctcgcgatgggtgacgccggcggttacaaggccgcagacatgtggggtccctcgagtgacccggcatgggagcgcaacgaccctacgcagcagatccccaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaacgccgcgggcgggcacaacgccgtgttcaacttcccgcccaacggcacgcacagctgggagtactggggcgctcagctcaacgccatgaagggtgacctgcagagttcgttaggcgccggc

SEQ ID NO: 61 - DNA, 39 bp, Artificial Sequence
  Synthetic Construct
caattacatatgggtacccatctcgccaacggttcgatg

SEQ ID NO: 62 - DNA, 33 bp, Artificial Sequence
  Synthetic Construct
caattagagctcgttgcacgcccagttgacgat

SEQ ID NO: 63 - DNA, 33 bp, Artificial Sequence
  Synthetic Construct
caattagagctcatgacctcgcgttttatgacg

SEQ ID NO: 64 - DNA, 33 bp, Artificial Sequence
  Synthetic Construct
caattagtcgacgctgctgaggatctgctggga

SEQ ID NO: 65 - DNA, 33 bp, Artificial Sequence
  Synthetic Construct
caattagtcgacatgaatttcgccgttttgccg

SEQ ID NO: 66 - DNA, 42 bp, Artificial Sequence
  Synthetic Construct
caattaaagcttttaagtactgaaaagtcggggtagcgccgg

SEQ ID NO: 67 - DNA, 30 bp, Artificial Sequence
  Synthetic Construct
caattacatatgaccatcaactatcaattc

SEQ ID NO: 68 - DNA, 33 bp, Artificial Sequence
  Synthetic Construct
caattaggtaccggcccagctggagccgacggc

(19) United States
(12) Patent Application Publication
(10) Pub. No.: US 2021/0253650 A1
(43) Pub. Date: Aug. 19, 2021

(54) COMPOSITIONS AND METHODS FOR TREATING SECONDARY TUBERCULOSIS AND NONTUBERCULOUS MYCOBACTERIUM INFECTIONS

(71) Applicant: Infectious Disease Research Institute, Seattle, WA (US)

(72) Inventor: Rhea N. COLER, Seattle, WA (US)

(21) Appl. No.: 17/241,494

(22) Filed: Apr. 27, 2021

Related U.S. Application Data
(62) Division of application No. 16/098,911, filed on Nov. 5, 2018, now abandoned.
(60) Provisional application No. 62/339,858, filed on May 21, 2016.

(51) Int. Cl. C07K 14/35; A61K 39/04; A61K 39/00

(52) U.S. Cl. CPC C07K 14/35; A61K 39/04; C07K 2319/40; A61K 2039/55561; C07K 2319/035

(57) ABSTRACT

Provided herein are fusion polypeptides comprising at least two Mycobacterial antigens, wherein one Mycobacterial antigen is a strong central memory T cell activator, and wherein one Mycobacterial antigen is a strong effector memory T cell activator. Also provided herein are methods of making and using such fusion polypeptides for the prevention or treatment of a secondary Mycobacterium tuberculosis infection as well as for the prevention or treatment of a nontuberculous Mycobacterium infection in a mammal.

### CROSS-REFERENCE TO RELATED APPLICATIONS

This application is a division of U.S. patent application Ser. No. 16/098,911, filed Nov. 5, 2018, which is a U.S. National Stage Application of No. PCT/US2017/033696, filed May 19, 2017 and claims the benefit of U.S. Provisional Application No. 62/339,858, filed May 21, 2016, the contents of the above referenced applications are hereby incorporated by reference in their entirety.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: 39-US-01_SEQLIST.TXT, date recorded: Mar. 19, 2021, size: 229 KB).

### BACKGROUND

Tuberculosis (TB) is a chronic infectious disease caused by infection with Mycobacterium tuberculosis (Mtb). TB is a major pandemic disease in developing countries, as well as an increasing problem in developed areas of the world, claiming between 1.7 and 2 million lives annually. Although infection may be asymptomatic for a considerable period of time, the disease is most commonly manifested as an acute inflammation of the lungs, resulting in fever and a nonproductive cough. If untreated, serious complications and death typically result. The increase of multidrug-resistant TB (MDR-TB) further heightens this threat (Dye, Nat Rev Microbiol 2009; 7:81-7).

Nontuberculous Mycobacterium (NTM) species cause a spectrum of disease including lung disease (TB-like), infections of the lymphatic system, skin, soft tissue, bone and systemic disease. There is a rise in NTM infections. Such infections, and especially such infections in immunocompromised patients, are creating an increasing reservoir for secondary infections in previously infected and drug treated Mtb infected individuals. There are currently over 150 different species of NTM but the more common infectious species are Mycobacterium avium complex (MAC), Mycobacterium kansasii, and Mycobacterium abscessus (reviewed in Nontuberculous mycobacterial pulmonary infections., Margaret M. Johnson and John A. Odell Journal of Thoracic Disease, Vol 6, No 3 Mar. 2014; The CDC (www.cdc.gov/nczved/divisions/dfbmd/diseases/nontb_mycobacterium/technical.html) notes that many NTM species that can be attributed to a variety of diseases including M. malmoense, M. simiae, M. szulgai, M. xenopi (associated with pneumonia); M. scrofulaceum (associated with lymphadenitis); and M. abscessus, M. chelonae, M. haemophilum, M. ulcerans (skin and soft tissue infections). In some tropical areas, Buruli ulcer disease caused by infection with M. ulcerans is a common cause of severe morbidity and disability.

The course of TB runs essentially through 3 phases. During the acute or active phase, the bacteria proliferate or actively multiply at an exponential, logarithmic, or semilogarithmic rate in the organs, until the immune response increases to the point at which it can control the infection whereupon the bacterial load peaks and starts declining. Although the mechanism is not fully understood, it is believed that sensitized CD4+ T cells in concert with interferon gamma (IFN-gamma, IFNγ) mediate control of the infection. Once the active immune response reduces the bacterial load and maintains it in check at a stable and low level, a latent phase is established. Previously, studies reported that during latency Mtb goes from active multiplication to dormancy, essentially becoming non-replicating and remaining inside the granuloma. However, recent studies have demonstrated that even in latency, at least part of the bacterial population remain in a state of active metabolism. (Talaat et al. 2007, J of Bact 189, 4265-74).

These bacteria therefore survive, maintain an active metabolism and minimally replicate in the face of a strong immune response. In the infected individual during latency there is therefore a balance between non-replicating bacteria (that may be very difficult for the immune system to detect as they are located intracellularly) and slowly replicating bacteria. In some cases, the latent infection enters reactivation, where the dormant bacteria start replicating again, albeit at rates somewhat lower than the initial infection. It has been suggested that the transition of Mtb from primary infection to latency is accompanied by changes in gene expression (Honerzu Bentrup, 2001). It is also likely that changes in the antigen-specificity of the immune response occur, as the bacterium modulates gene expression during its transition from active replication to dormancy. The full nature of the immune response that controls latent infection and the factors that lead to reactivation are largely unknown. However, there is some evidence for a shift in the dominant cell types responsible. While CD4+ T cells are essential and sufficient for control of infection during the acute phase, some studies suggest that CD8+ T cell responses are more important in the latent phase. Bacteria in this stage are typically not targeted by most of the preventive vaccines that are currently under development in the TB field, as exemplified by the lack of activity when classical preventive vaccines are given to latently infected experimental animals (Turner et al. 2000 Infect Immun. 68:6:3674-9.).

Unlike the diagnosis of TB caused by Mtb species, where isolation of the bacterium in a clinical specimen is diagnostic for disease, the presence of a NTM species in a clinical isolate does not correlate with disease. NTMs share many characteristics with the Mtb species that make the bacteria difficult to differentiate in resource-poor settings. The standard method for diagnosting TB is through microscopic examination of sputum smears, but when this approach is used, NTMs appear identical to Mtb. Without molecular methods, these organisms are difficult to distinguish. Patients are often assumed to have Mtb infections because the clinical manifestations of many NTMs can mimic those of TB. The American Thoracic Society (AT S) and the Infectious Disease Society of America (IDSA) jointly published guidelines in 2007 requires the presence of symptoms, radiologic abnormalities, and microbiologic cultures in conjunction with the exclusion of other potential etiologies in order to diagnose NTM pulmonary infection (M. Johnson and John A. Odell Journal of Thoracic Disease, Vol 6, No 3 Mar. 2014). Many NTM species are found in drinking water, household plumbing, peat rich soils, brackish marshes, drainage water, water systems in hospitals, hemodialysis centers, and dental offices making them particularly ubiquitous in the environment.

Although Mtb can generally be controlled using extended antibiotic therapy, such treatment is not sufficient to prevent the spread of the disease. Infected individuals may be asymptomatic, but contagious, for some time. Current clinical practice for latent TB (asymptomatic and non-contagious) is treatment with 6 to 9 months of isoniazid or other antibiotic or, alternatively, treatment with 4 months of rifampin. Active Mtb infection is treated with a combination of 4 medications for 6 to 8 weeks during which the majority of bacilli are thought to be killed, followed by two drugs for a total duration of 6 to 9 months. Duration of treatment depends on the number of doses given each week. In addition, although compliance with the treatment regimen is critical, patient behavior is difficult to monitor. Some patients do not complete the course of treatment either due to side effects or the extreme duration of treatment (6-9 months), which studies have shown can lead to ineffective treatment and the development of drug resistance. In addition, there is increasing concern that the rise antibiotic resistant strains, especially multidrug resistant (MDR) strains of Mtb species can lead to an increase in the emergence of drug resistant NTM species. Standard TB treatments are often ineffective against NTM infections. Anti-TB medications produce a response rate of approximately 50% in NTM-associated disease.

Regardless of the chronology of causality of secondary tuberculosis disease and NTM infection, the risk of the increasing incidence of TB disease and the emergence MDR strains of Mtb species and NTM species is a serious health concern for the developing and developed world. Thus, in order to decrease TB transmission globally, and decrease the emergence of drug resistant and multidrug resistant Mtb and NTM species, there is an urgent need for more effective prophylactic and therapeutic treatments of secondary Mtb infections, and infection with NTM species. The methods and compositions provided herein are useful for treating and preventing secondary Mtb infections, and for preventing and treating NTM infections.

### SUMMARY OF THE INVENTION

The present disclosure provides compositions and methods for preventing or treating secondary tuberculosis (TB) caused by Mtb in a subject as well as compositions and methods for preventing or treating infections caused by NTM in a subject, including the treatment of subjects with pre-existing structural pulmonary disease (e.g., subjects with a history of prior TB, chronic obstructive pulmonary disease or cystic fibrosis).

The compositions and methods described herein for treating TB are capable of eliciting both a strong central memory T cell response and a strong effector memory T cell response. Provided herein are methods of administering any one of the fusion polypeptides described herein. Such fusion polypeptpides comprise at least two Mycobacterial antigens, wherein one antigen is a strong central memory T cell activator, and wherein one antigen is a strong effector memory T cell activator.

In one aspect, provided herein are fusion polypeptides comprising at least two Mycobacterial antigens, wherein one antigen is a strong central memory T cell activator, and wherein one antigen is a strong effector memory T cell activator. In some embodiments, the strong central memory T cell activator antigen comprises a sequence having at least 90% sequence identity to Rv1813-b, Rv2608b, Rv2389-b, or Rv1886-b. In some embodiments, the strong central memory T cell activator antigen comprises the sequence of Rv1813-b, Rv2608b, Rv2389-b, or Rv1886-b. In some embodiments, the strong effector memory T cell activator antigen comprises a sequence having at least 90% sequence identity to Rv3619 or Rv3620. In some embodiments, the strong effector memory T cell activator antigen comprises the sequence of Rv3619 or Rv3620. In some embodiments, the fusion polypeptide further comprises a third antigen, wherein the third antigen is a strong central memory T cell activator. In some embodiments, the fusion polypeptide further comprises a third antigen, wherein the third antigen is a strong effector memory T cell activator. In some embodiments, the fusion polypeptide comprises antigens having at least 90% sequence identity to Rv3619, Rv3620, Rv2389-b, and Rv2608-b. In some embodiments the fusion polypeptide comprises Rv3619, Rv3620, Rv2389-b, and Rv2608-b. In some embodiments, the fusion polypeptide has at least a 90% sequence identity to ID93-1, ID93-2, ID83-1, ID83-2, or ID97. In some embodiments, the fusion polypeptide is ID93-1, ID93-2, ID83-1, ID83-2, or ID97. In some embodiments, the fusion polypeptide is ID91.

In another aspect provided herein are pharmaceutical compositions comprising any one of the fusion polypeptides provided herein, and a pharmaceutically acceptable carrier, excipient, or diluent.

In another aspect, provided herein is a method of activating a strong Mycobacterial central memory T cell response and a strong Mycobacterial effector memory T cell response in a subject comprising administering to a subject an effective amount of any one of the fusion polypeptides or pharmaceutical compositions comprising the fusion polypeptides provided herein. In some embodiments, the subject is Quantiferon positive. In some embodiments, the subject is Quantiferon negative.

In another aspect, provided herein is a method of preventing or treating secondary tuberculosis infection in a subject, comprising administering to a subject an effective amount of any one of the fusion polypeptides or pharmaceutical compositions comprising the fusion polypeptides provided herein. In some embodiments, the method is used for preventing secondary tuberculosis infection in a subject. In some embodiments, the method is used for treating secondary tuberculosis infection in a subject. In some embodiments, the tuberculosis infection is reactivation of a latent Mtb infection. In some embodiments, the lung infection is reactivation of a latent NTM infection. The subject can be Quantiferon positive or Quantiferon negative.

In another aspect, provided herein is a method of preventing or treating a nontuberculous Mycobacterium (NTM) infection in a subject, comprising administering to a subject an effective amount of any one of the fusion polypeptides or pharmaceutical compositions comprising the fusion polypeptides provided herein. In some embodiments, the method is used for preventing NTM infection in a subject. In some embodiments, the method is used for treating NTM infection in a subject. In some embodiments, the NTM infection is a primary infection. In some embodiments, the NTM infection is a secondary infection. The subject can be Quantiferon positive or Quantiferon negative.

In another aspect, provided herein is a method of reducing a sign or symptom of an active TB disease in a subject, comprising administering to a subject an effective amount of any one of the fusion polypeptides or pharmaceutical compositions comprising the fusion polypeptides provided herein. In some embodiments, the subject is Quantiferon positive. In some embodiments, the subject is Quantiferon negative.

In another aspect, provided herein is a method of preventing or treating a nontuberculous Mycobacterium (NTM) infection in a subject, comprising administering to a subject an effective amount of a fusion polypeptide that has at least a 90% sequence identity to ID93-1, ID93-2, ID83-1, ID83-2, ID97 or ID91 or that is ID93-1, ID93-2, ID83-1, ID83-2, ID97 or ID91.

In another aspect, provided herein is a method of reducing NTM bacterial burden in a subject comprising contacting a cell of the subject with (i) a TLR 4 agonist, (ii) a fusion polypeptide that has at least a 90% sequence identity to ID93-1, ID93-2, ID83-1, ID83-2, ID97 or ID91 or (iii) a combination thereof.

It is to be understood that one, some, or all of the properties of the various embodiments described herein may be combined to form other embodiments of the present invention. These and other aspects of the invention will become apparent to one of skill in the art.

### BRIEF DESCRIPTION OF THE DRAWINGS

FIG. 1 shows the kinetics of ID93 antigen-specific CD4⁺ T cells measured at baseline and 2 weeks after vaccination. Frequencies of CD4+ T cells positive for any antigen-specific marker (IFNg, TNF, IL-2, CD154, IL-22 and/or IL-17) as measured by intracellular cytokine staining of antigen (peptide pools)-stimulated PBMCs with unstimulated values subtracted. Vaccinations were administered on days 0, 28, and 56.

FIG. 2 shows the median total quantitative changes in CD4+ T cell responses of whole blood to stimulation with pools containing Rv1813 (either Rv1813-a or Rv1813-b), Rv2608 (either Rv2608-a or Rv2608-b), Rv3619 or Rv3620 peptides/antigens. Error bars represent inter-quartile ranges (IQR) for each stimulation. ID93-2 vaccinated and placebo subjects are stratified by Cohort, and responses stratified longitudinally by study day. Background values (unstimulated) were subtracted. Data demonstrates that immunization with ID93-2 generates an immune response in vaccinated subjects that peaks at day 14-42 days post immunization overall. Rv2608 (3^{rd} stacked bar from the top) and Rv3619 (second stacked bar from the top) generate the quantitatively highest immune response to the antigenic subunit proteins of ID93-2. The post ID93+GLA-SE vaccination magnitude and kinetics of responses to each specific antigen varied among the cohorts. Vaccination induced an Rv2608-specific CD4 T cell response that was higher than baseline in all ID93+GLA-SE vaccinated participants, irrespective of cohort. In all cohorts, maximal CD4 T cell responses to Rv1813 and Rv2608 were seen after two administrations of vaccine, irrespective of dosage. A third administration of vaccine did not appreciably boost responses above those seen by second administration. In Cohorts 2, 3 and 4, a single administration of ID93+GLA-SE rapidly induced a CD4 T cell response to Rv3619 and Rv3620, most likely as a boost effect to underlying latent M. tb infection. However, in the QFT-negative Cohort 1 participants, Rv3619 and Rv3620 responses post vaccination were not statistically different from baseline (e.g., Wilcoxon p values for Rv3619 and Rv3620 at Day 42, the peak measured response, were 0.9453 and 0.6875, respectively) or placebo (Mann-Whitney) responses, suggesting that responses to Rv3619 and Rv3620 were not inducible by ID93+GLA-SE in individuals not otherwise primed by natural infection with M. tb.

FIG. 3 depicts the general method for performing an FDS Analysis.

FIG. 4A-B shows the FDS qualitative analysis of CD4+ T cell populations in subjects vaccinated with ID93-2+GLA-SE from cohorts 2 and 4 of the clinical study in both QFT+(previously infected with a TB-causing pathogen subjects, left panel, Quantiferon positive) and QFT-(TB naïve, right panel, Quantiferon negative) subjects after intracellular staining analysis of PBMCs stimulated with the antigenic subunits proteins of ID93-2 (Rv1813 (a or b), Rv2608 (a or b), Rv3619, and Rv3620). The data show that Rv1813 and 2608 are strong central memory CD4+ antigens and that vaccination of naïve tuberculosis subjects with ID93-2 can drive differentiation of T cell profiles to strong central memory responses (FDS score 1 or less) to these antigens. Conversely the Rv3619 and Rv3620 are strong effector memory CD4+ antigens (FDS score 3 or greater) and that vaccination of naïve tuberculosis subjects with ID93-2 can drive differentiation of CD4+ T cell profiles to strong effector memory responses to these antigens.

FIG. 5A-B shows the FDS profiles 6 months after the final vaccination with ID93-2 in subjects immunized with ID93-2+GLA-SE from Cohorts 2 and 4 of the clinical trial. The data in FIG. 5A shows an analysis of the FDS score for the ID93-2 subunit proteins in different TB populations. Six months after vaccination with three doses of ID93-2+GLA-SE, in both QFT+ and QFT-subjects, the data show that Rv2608 and Rv1813 proteins of ID93-2 are strong CD4+ T cell central memory antigens and that Rv3619 and Rv3620 proteins of ID93-2 are strong CD4+ T cell effector memory antigens. FIG. 5B shows that overall, Rv2608 and Rv1813 are strong CD4+ T cell central memory antigens and Rv3619 and Rv3620 are both strong CD4+ T cell effector memory antigens, regardless of the population's tuberculosis status.

FIG. 6 shows Growth inhibition of the NTM M. Avium by GLA-AF and QS21. TLR4 formulations inhibit growth of the NTM M. Avium mycobacteria.

FIG. 7 shows growth inhibiton of of the NTM M. Avium by ID91-GLA-SE or ID91.

### DETAILED DESCRIPTION

The present disclosure provides compositions and methods for preventing or treating secondary tuberculosis (TB) caused by Mtb. The disclosure also provides compositions and methods for preventing or treating primary and secondary infections caused by NTM, including pulmonary infections that mimic TB. In exemplary embodiments, the compositions and methods for treating such TB and NTM infections are capable of eliciting both a strong central memory T cell response and a strong effector memory T cell response upon administration with any one of the fusion polypeptides provided herein comprising at least two Mycobacterial antigens, wherein one antigen is a strong central memory T cell activator, and wherein one antigen is a strong effector memory T cell activator.

The present disclosure is based, inter alia, on the surprising discovery that certain Mycobacterium antigens are capable of activating a strong Mycobacterial central memory T cell response and certain Mycobacterium antigens are capable of activating a strong Mycobacterial effector memory T cell response. Likewise, it was a surprising discovery administration of a fusion polypeptide comprising at least two Mycobacterial antigens, wherein one antigen is a strong central memory T cell activator and one antigen is a strong effector memory T cell activator to a subject elicited both a strong Mycobacterial central memory T cell response and a strong Mycobacterial effector memory T cell response.

The present disclosure is also based, inter alia, on the discovery that the described Mycobacterium antigens are capable of preventing or treating TB in a subject that has already had TB and been successfully treated for TB (e.g., previously infected subjects).

As described herein, the present disclosure relates generally to compositions and methods for preventing or treating secondary tuberculosis disease (TB) in a subject, and for preventing or treating a nontuberculous Mycobacterium (NTM) infection in a subject, the methods comprising administering to the subject an effective amount of a fusion polypeptide comprising at least two Mycobacterial antigens. In exemplary embodiments, one antigen is a strong central memory T cell activator and wherein one antigen is a strong effector memory T cell activator.

As described herein, TLR4 agonists can also be used to prevent or treat a nontuberculous Mycobacterium (NTM) infection in a subject. Provided herein are methods comprising administering to the subject an effective amount of TLR4 agonist for the treatment of NTM infection. Also provided are methods of reducing NTM bacterial burden in a subject comprising contacting a cell of the subject with (i) a TLR4 agonist (ii) any of the fusion polyeptides described herein or (iii) a combination thereof. The subject's cell can be in the subject and contacting is via administering the TRL4 agonist and/or any of the fusion polypeptides described herein to the subject.

### Definitions

In the present description, the terms “about” and “consisting essentially of” mean±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

An “individual” or a “subject” is a mammal, e.g., a human mammal or a non-human mammal. Non-human mammals include, but are not limited to, farm animals (such as cattle, pigs, horses), sport animals, pets (such as cats, dogs, horses), primates, mice and rats.

“M. tuberculosis” and “Mtb” refer to the bacterium of type, Mycobacterium tuberculosis, that can cause TB disease in a mammal.

“Nontuberculous Mycobacterium” or “NTM” as used interchangeably herein includes those bacterial species that can cause NTM related infections in mammals including pulmonary infection, e.g., pulmonary infection that mimics TB. NTMs are defined as any mycobacterial pathogen other than Mtb orMycobacterium leprae. NTMs cause a spectrum of disease that include pulmonary infection (e.g., TB-like lung disease), infections of the lymphatic system, skin, soft tissue, or bone, and systemic disaease. NTMs can infect, for example, subjects with no pre-existing disease, subjects with with pre-existing structural pulmonary disease (e.g. subjects with a history of prior TB, chronic obstructive pulmonary disease or cystic fibrosis) as well as immune compromised patients, such as patients with AIDS, and patients that have had a primary Mtb infection. NTMs include, but are not limited to, M. bovis, or M. africanum, BCG, M. avium, M. intracellulare, M. celatum, M. genavense, M. haemophilum, M. kansasii, M. abscessus, M. simiae, M. vaccae, M. fortuitum, and M. scrofulaceum species (see, e.g., Harrison's Principles of Internal Medicine, Chapter 150, pp. 953-966 (16th ed., Braunwald, et al., eds., 2005). Many NTM species are found in drinking water, household plumbing, peat rich soils, brackish marshes, drainage water, water systems in hospitals, hemodialysis centers, and dental offices making them particularly ubiquitous in the environment.

As used herein, a “Mycobacterial infection” or “infection with a Mycobacterium” refers to infection with a Mtb and/or a NTM.

As used herein, a “Mycobacterial antigen” refers to an antigen from Mtb or a NTM. As used herein, a “Mtb antigen” refers to an antigen from Mtb.

As used herein, a “NTM antigen” refers to an antigen from a NTM, for example an antigen from M. avium, M. kansasii, M. bovis, M. intracellulare, M. celatum, M. malmoense, M. simiae, M. szulgai, M. xenopi (associated with pneumonia); M. scrofulaceum (associated with lymphadenitis); and M. abscessus, M. chelonae, or M. haemophilum, or M. ulcerans.

“Primary Tuberculosis” or “primary TB” or a “primary TB infection” or a “primary Tuberculosis infection” or “primary infection” or a “primary Mycobacterial infection” as used herein refers to a TB disease that develops within the first several years after initial exposure to and infection with a Mycobacterium Tuberculosis, due to failure of the host immune system to adequately contain the initial infection. Some primary infections are never treated.

“Secondary Tuberculosis” or “secondary TB” or a “secondary TB infection” or a “secondary Tuberculosis infection” or a “secondary infection” a “secondary Mycobacterial infection” as used herein refers to (i) a TB disease that occurs due to reactivation of a latent strain from a primary Mtb infection, (ii) a TB disease that occurs due to a second subsequent reinfection with a second Mtb strain, wherein the strain responsible for the primary Mtb infection and the strain responsible for the secondary Mtb infection are not the same strains or (iii) A TB disease characterized both by reactivation of a latent strain from a primary Mtb infection and a second subsequent reinfection with second Mtb strain

Secondary TB includes infection of a host with a secondary Mycobacterial strain not identified in primary clinical isolates. Secondary TB also includes isolates present at an increased frequency in the secondary clinical isolate compared to the Primary TB isolates. Secondary TB can occur for example in a host that has a latent TB infection.

As used herein, a “NTM infection” refers to either a primary or a secondary infection with a NTM.

A “drug resistant” Mycobacterial infection refers to a Mtb infection or infection with a nontuberculous Mycobacterium (NTM) wherein the infecting strain is not held static or killed (is resistant to) one or more of so-called “frontline” chemotherapeutic agents effective in treating a Mtb or NTM infection (e.g., isoniazid, rifampin, ethambutol, streptomycin, and pyrazinamide).

A “multi-drug resistant” infection refers to a Mtb or NTM infection wherein the infecting strain is resistant to two or more of “front-line” chemotherapeutic agents effective in treating a Mtb or NTM infection. Multi-drug resistant infections as used herein also refer to “extensively drug-resistant tuberculosis” (“XDR-TB”) as defined by the World Health Global task Force in October 2006 as a multi-drug resistant TB with resistance to any one of the fluoroquinolones (FQs) and at least one of the injectable drugs such as kanamycin, amikacin, and capreomycin.

“Active Tuberculosis”, “Active TB”, “TB Disease”, “TB” or “Active TB Infection” as used herein refers to an illness, condition, or state in a mammal (e.g., a primate such as a human) in which Mtb bacteria are actively multiplying and invading organs of the mammal and causing symptoms or about to cause signs, symptoms or other clinical manifestations, most commonly in the lungs (pulmonary active TB). Clinical symptoms of active TB may include weakness, fatigue, fever, chills, weight loss, loss of appetite, anorexia, or night sweats. Pulmonary active TB symptoms include cough persisting for several weeks (e.g., at least 3 weeks), thick mucus, chest pain, and hemoptysis. “Reactivation tuberculosis” as used herein refers to active TB that develops in an individual having LTBI and in whom activation of dormant foci of infection results in actively multiplying Mtb bacteria. “Actively multiplying” as used herein refers to Mtb bacteria which proliferate, reproduce, expand or actively multiply at an exponential, logarithmic, or semilogarithmic rate in the organs of an infected host. In certain embodiments, an infected mammal (e.g., human) has a suppressed immune system. The immune suppression may be due to age (e.g., very young or older) or due to other factors (e.g., substance abuse, organ transplant) or other conditions such as another infection (e.g., HIV infection), diabetes (e.g., diabetes mellitus), silicosis, head and neck cancer, leukemia, Hodgkin's disease, kidney disease, low body weight, corticosteroid treatment, or treatments for arthritis (e.g., rheumatoid arthritis) or Crohn's disease, or the like.

Tests for determining the presence of lung disease caused by Mtb or NTM bacteria or condition caused by actively multiplying Mtb or NTM bacteria are known in the art and include but are not limited to Acid Fast Staining (AFS) and direct microscopic examination of sputum, bronchoalveolar lavage, pleural effusion, tissue biopsy, cerebrospinal fluid effusion; bacterial culture such as the BACTEC MGIT 960 (Becton Dickinson, Franklin Lakes, N.J., USA); IGR tests including the QFT®-Gold, or QFT®-Gold In-tube T SPOTT M. TB, skin testing such as the TST The Mantoux skin test (TST); and intracellular cytokine staining of whole blood or isolated PBMC following antigen stimulation. The American Thoracic Society (ATS) and the Infectious Disease Society of America (IDSA) jointly published guidelines in 2007 requires the presence of symptoms, radiologic abnormalities, and microbiologic cultures in conjunction with the exclusion of other potential etiologies in order to diagnose NTM pulmonary infection (M. Johnson and John A. Odell Journal of Thoracic Disease, Vol 6, No 3 Mar. 2014).

“Latent Infection”, “Latency”, or “Latent Disease”, “Dormant Infection”, as used herein refers to an infection with Mtb or NTM that has been contained by the host immune system resulting in a dormancy which is characterized by constant low bacterial numbers but may also contain at least a part of the bacterial population which remains in a state of active metabolism including reproduction at a steady maintenance state. Latent TB infection is determined clinically by a positive TST or IGRA without signs, symptoms or radiographic evidence of active TB disease. Latently infected mammals are not “contagious” and cannot spread disease due to the very low bacterial counts associated with latent infections. Latent tuberculosis infection (LTBI) is treated with a medication or medications to kill the dormant bacteria. Treating LTBI greatly reduces the risk of the infection progressing to active tuberculosis (TB) later in life (e.g., it is given to prevent reactivation).

A “method of prevention” or “method of preventing” as disclosed herein, refers generally to a method for preventing secondary TB or NTM infection in a mammal using a prophylactic composition (e.g., a prophylactic vaccine). Typically, the initial step of administering the prophylactic composition will occur before the subject is infected with Mtb or an NTM, and/or before the subject exhibits any clinical symptom or positive assay result associated with infection.

A “method of treatment” or “method of treating” as disclosed herein, refers generally to a method for treating secondary TB or NTM infection (primary NTM infection or secondary NTM infection) in a subject using a therapeutic composition (e.g. a therapeutic vaccine) either alone or in conjunction with a chemotherapeutic treatment regime. It will be understood in this and related methods of the disclosure that at least one step of administering the therapeutic composition, typically the initial step of administering the therapeutic composition will take place when the mammal is actively infected with Mtb or an NTM and/or exhibits at least one clinical symptom or positive assay result associated with active infection. It will also be understood that the methods of the present disclosure may further comprise additional steps of administering the same or another therapeutic composition of the present disclosure at one or more additional time points thereafter, irrespective of whether the active infection or symptoms thereof are still present in the subject, and irrespective of whether an assay result associated with active infection is still positive, in order to improve the efficacy of chemotherapy regimens. It will also be understood that the methods of the present disclosure may include the administration of the therapeutic composition either alone or in conjunction with other agents and, as such, the therapeutic composition may be one of a plurality of treatment components as part of a broader therapeutic treatment regime.

A “chemotherapeutic”, “chemotherapeutic agents” or “chemotherapy regime” is a drug or combination of drugs used to treat or in the treatment thereof of patients infected or exposed to any TB-causing Mycobacterium and includes, but is not limited to, amikacin, aminosalicylic acid, capreomycin, cycloserine, ethambutol, ethionamide, isoniazid (INH), kanamycin, pyrazinamide, rifamycins (i.e., rifampin, rifapentine and rifabutin), streptomycin, ofloxacin, ciprofloxacin, clarithromycin, azithromycin and fluoroquinolones and other derivatives analogs or biosimilars in the art. “First-line” chemotherapeutic agents are chemotherapeutic agents used to treat a Mycobacterium infection that is not drug resistant and include, but are not limited to, isoniazid, rifampin, ethambutol, streptomycin and pyrazinamide and other derivatives analogs or biosimilars in the art. “Second-line” chemotherapeutic agents used to treat a Mycobacterium infection that has demonstrated drug resistance to one or more “first-line” drugs include without limitation ofloxacin, ciprofloxacin, ethionamide, aminosalicylic acid, cycloserine, amikacin, kanamycin and capreomycin and other derivatives analogs or biosimilars in the art.

As used herein “improving the efficacy of chemotherapy regimens” refers to shortening the duration of therapy required to achieve a desirable clinical outcome, reducing the number of different chemotherapeutics required to achieve a desirable clinical outcome, reducing the dosage of chemotherapeutics required to achieve a desirable clinical outcome, decreasing the pathology of the host or host organs associated with an active clinical infection, improving the viability of the host or organs of a host treated by the methods, reducing the development or incidence of MDR-TB strains, and or increasing patient compliance with chemotherapy regimens.

Therapeutic TB compositions as provided herein refer to a composition(s) capable of eliciting a beneficial immune response to a Mycobacterium infection when administered to a host with an active TB infection. A “beneficial immune response” is one that lessens signs or symptoms of active TB disease, reduces bacillus counts, reduces pathology associated with active TB disease, elicits an appropriate cytokine profile associated with resolution of disease, expands antigen specific CD4⁺ and CD8⁺ T cells, or improves the efficacy of chemotherapy regimens. Therapeutic TB compositions as provided herein refer to a composition(s) capable of eliciting an immune response in a subject such as an increase in the overall quantitative number of antigen specific T cells or a qualitative change in the differentiation state of the T cells of a subject which can be measured empirically by the methods of the invention or by the generation of a beneficial immune response (e.g., reduction in signs of symptoms).

Therapeutic TB compositions of the disclosure include without limitation antigens, fusion polypeptides, and polynucleotides which encode antigens and fusion polypeptides which are delivered in a pharmaceutically acceptable formulation by methods known in the art.

As used herein “FDS” refers to a functional differentiation score. An FDS is calculating by the following formula: [% IFN-γ+CD4+ T cells/% IFN-γ-CD4+ T cells].

“IFN-γ+CD4+ T cells” are CD4+ T cells that produce IFN-γ. For example, such cells show intracellular staining of IFN-γ as measured by methods known in the art including Intracellular Cytokine Staining (ICS), or secrete IFN-γ as measured by methods known in the art including ELISAs.

“IFN-γ-CD4+ T cells” are CD4+ T cells that do not produce IFN-γ. For example, such cells do not show intracellular staining of IFN-γ, as measured by methods known in the art, including ICS, and do not secrete IFN-γ, as measure by methods known in the art including ELISAs.

An FDS can be used to: (1) to measure qualitative changes in the CD4+ T cell profile status of a subject to one or more antigens (e.g. a composition, formulation or vaccine comprising the antigen(s)); (2) to qualify the quantitative changes in the percent of CD4+ T cells at baseline (t=0) or following administration of one or more antigens (e.g. a composition, formulation or vaccine comprising the antigen(s)); and (3) to analyze the qualitative changes in CD4+ T cell profile status to one or more antigens (e.g. a composition, formulation or vaccine comprising the antigen(s)) in an overall population (regardless of TB status, e.g. such as individuals previously infected or exposed to TB-causing bacteria or naive individuals never infected with TB-causing bacteria; or for e.g. in a QFT− or QFT+ or mixed populations).

As used herein a “strong central memory T cell response” is elicited when the FDS of a subject is less than or equal to about 1.0, after one or more immunizations.

As used herein a “strong effector memory T cell activator response” is elicited when the FDS of a subject is more than or equal to about 3.0, after one or more immunizations.

A low FDS represents cells in early stages of T cell differentiation or expansion of central memory T cells, whereas a high FDS indicates greater differentiation or expansion of effector T cells.

Fusion Polypeptide Compositions

Provided herein are Mycobacterial antigens capable of eliciting strong central memory T cell responses and Mycobacterial antigens capable of eliciting strong effector memory T cell responses. Also provided herein are fusion polypeptides comprising at least two Mycobacterial antigens, wherein one antigen is a strong central memory T cell activator, and wherein one antigen is a strong effector memory T cell activator for treating secondary TB infections and NTM infections.

The fusion polypeptides provided herein may comprise at least two, at least three, at least four, at least five, at least six, at least seven, at least eight, at least nine, or even at least ten Mycobacterial antigens, wherein the fusion polypeptide is capable of eliciting strong central memory and effector memory T cell responses upon administration.

Fusion polypeptides and Mycobacterial antigens may be prepared using conventional recombinant and/or synthetic techniques.

Also provided herein are assays and methods for the screening of selection of Mycobacterial antigens capable of eliciting both a strong central memory T cell response, and a strong effector memory T cell response.

Provided herein are Mtb and NTM antigens and fusion polypeptides comprising at least two antigens. Fusion polypeptides to a polypeptide having at least two heterologous Mycobacterium antigens, such as Mtb antigens and/or NTM antigens. In the fusion polypeptides provided herein, the individual antigens may be covalently linked, either directly or indirectly via an amino acid linker. The linker may range from 1 amino acid in length to 100 amino acids in length. The individual antigens forming the fusion polypeptide are typically linked C-terminus to N-terminus, although they can also be linked C-terminus to C-terminus, N-terminus to N-terminus, or N-terminus to C-terminus. The antigens may be linked in any order, regardless of presentation or recitation.

The fusion polypeptides can also include conservatively modified variants, polymorphic variants, alleles, mutants, subsequences, interspecies homologs, and immunogenic fragments of the antigens that make up the fusion protein. Mtb antigens are described in Cole et al., Nature 393:537 (1998), which discloses the entire Mycobacterium tuberculosis genome. Antigens from other NTM species can be identified, e.g., using sequence comparison algorithms, as described herein, cross reactivity assays, or other methods known to those of skill in the art, e.g., hybridization assays and antibody binding assays.

The fusion polypeptides of the disclosure generally comprise at least two antigenic polypeptides as described herein, and may further comprise other unrelated sequences, such as a sequence that assists in providing T helper epitopes (an immunological fusion partner), T helper epitopes recognized by humans, or that assists in expressing the protein (an expression enhancer) at higher yields than the native recombinant protein. Certain exemplary fusion partners are both immunological and expression enhancing fusion partners. Other fusion partners may be selected so as to increase the solubility of the protein or to enable the protein to be targeted to desired intracellular compartments. Still further fusion partners include affinity tags, which facilitate purification of the protein.

Fusion proteins may generally be prepared using standard techniques. In some embodiments, a fusion protein is expressed as a recombinant protein. For example, DNA sequences encoding the polypeptide components of a desired fusion may be assembled separately and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and second antigen (or subsequent antigens) by a distance sufficient to ensure that each antigen folds into its secondary and tertiary structures, if desired. Such a peptide linker sequence is incorporated into the fusion protein using standard techniques well known in the art. Certain peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. In some embodiments, the peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39 46 (1985); Murphy et al., Proc. Natl. Acad. Sci. USA 83:8258 8262 (1986); U.S. Pat. Nos. 4,935,233 and 4,751,180. The linker sequence may generally be from 1 to about 100 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

Within some embodiments, an immunological fusion partner for use in a fusion polypeptide of the disclosure is derived from protein D, a surface protein of the gram-negative bacterium Haemophilus influenza B (WO 91/18926). In some embodiments, a protein D derivative comprises approximately the first third of the protein (e.g., the first N-terminal 100 110 amino acids), and a protein D derivative may be lipidated. Within certain some embodiments, the first 109 residues of a lipoprotein D fusion partner is included on the N-terminus to provide the fusion polypeptide with additional exogenous T cell epitopes and to increase the expression level in E. coli (thus functioning as an expression enhancer). The lipid tail ensures optimal presentation of the antigen to antigen presenting cells. Other fusion partners include the non-structural protein from influenza virus, NS 1 (hemaglutinin). Typically, the N-terminal 81 amino acids are used, although different fragments that include T-helper epitopes may be used.

In another embodiment, an immunological fusion partner comprises an amino acid sequence derived from the protein known as LYTA, or a portion thereof (for e.g., a C-terminal portion). LYTA is derived from Streptococcus pneumoniae, which synthesizes an N-acetyl-L-alanine amidase known as amidase LYTA (encoded by the LytA gene; Gene 43:265-292 (1986)). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798 (1992)). Within an exemplary embodiment, a repeat portion of LYTA may be incorporated into a fusion protein. A repeat portion is found in the C-terminal region starting at residue 178. An exemplary repeat portion incorporates residues 188-305.

In general, antigens and fusion polypeptides (as well as their encoding polynucleotides) are isolated. An “isolated” polypeptide or polynucleotide is one that is removed from its original environment. For example, a naturally-occurring protein is isolated if it is separated from some or all of the coexisting materials in the natural system. In some embodiments, such polypeptides are at least about 90% pure, at least about 95% pure or even about 99% pure. A polynucleotide is considered to be isolated if, for example, it is cloned into a vector that is not a part of the natural environment.

Sequences of exemplary Mycobacterial antigens are provided in Table 1. Sequences of exemplary fusion polypeptides are provided in Table 2. In some embodiments, the present disclosure provides variants of the sequences described herein. Polypeptide variants generally encompassed by the present disclosure will typically exhibit at least about 70%, 75%, 80%, 85%, 90%, 91%, 92%, 93%, 94%, 95%, 96%, 97%, 98%, or 99% or more sequence identity, along its length, to a polypeptide sequence set forth herein. A polypeptide “variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the disclosure and evaluating their immunogenic activity as described herein using any of a number of techniques well known in the art.

For example, certain illustrative variants of the polypeptides of the disclosure include those in which one or more portions, such as an N-terminal leader sequence or transmembrane domain, have been removed. Other illustrative variants include variants in which a small portion (e.g., about 1-30 amino acids) has been removed from the N- and/or C-terminal of a mature protein.

In many instances, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. For example, certain amino acids may be substituted for other amino acids in a protein structure without appreciable loss of interactive binding capacity. In making such changes, the hydropathic index of amino acids may be considered. Amino acid substitutions may further be made on the basis of similarity in polarity, charge, solubility, hydrophobicity, hydrophilicity and/or the amphipathic nature of the residues.

A variant may also, or alternatively, contain nonconservative changes. In an exemplary embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

As noted above, polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

When comparing polypeptide sequences, two sequences are said to be “identical” if the sequence of amino acids in the two sequences is the same when aligned for maximum correspondence, as described below.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Nat'l Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Nat'l Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the disclosure. Software for performing BLAST analyses is publicly available through the National Center for Biotechnology Information.

In one approach, the “percentage of sequence identity” is determined by comparing two optimally aligned sequences over a window of comparison of at least 20 positions, wherein the portion of the polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e., the window size) and multiplying the results by 100 to yield the percentage of sequence identity.

Exemplary Fusion Polypeptides

Provided herein, inter alia, are fusion polypeptides comprising at least two Mycobacterial antigens, wherein one antigen is a strong central memory T cell activator, and wherein one antigen is a strong effector memory T cell activator. In some embodiments, the fusion polypeptides further comprise additional Mycobacterial antigens, for example the fusion polypeptides comprise two, three, four, five, six, seven, eight, nine, or even ten Mycobacterial (either Mtb or NTM) antigens.

Exemplary Mycobacterial antigens are provided in Table 1. It is to be noted that throughout the entirety of the disclosure, including the Drawings, Examples and Claims, when referring to the antigens of the invention, if a specific suffix is not used, for example if simply “Rv1813” is referred to, such use refers to either or both 1813-a and 1813-b.

Exemplary fusion polypeptides are provided in Table 2. It is to be noted that throughout the entirety of the disclosure, including the Drawings, Examples and Claims, when referring to the fusion polypeptides of the invention, if a specific suffix is not used, for example if simply “ID93” is referred to, such use refers to either or both ID93-1 and ID93-2.

In some embodiments, the strong Mycobacterial central memory T cell activator antigen comprises a sequence that has cross reactivity with an NTM antigen.

In some embodiments, the strong Mycobacterial effector memory T cell activator antigen comprises a sequence that has cross reactivity with an NTM antigen.

In some embodiments, the strong Mycobacterial central memory T cell activator antigen comprises a sequence having at least 90% sequence identity to Rv1813-a, Rv1813-b, Rv1886-a, Rv1886-b, Rv2389-a, Rv2389-b, Rv2608-a, or Rv2608-b. In some embodiments, the strong Mycobacterial central memory T cell activator antigen comprises a sequence having at least 90% sequence identity to Rv1813-b, Rv2389-b, Rv1886b, or Rv2608-b. In some embodiments, the strong Mycobacterial central memory T cell activator antigen comprises a sequence having at least 90% sequence identity to Rv1813-a, Rv1813-b, Rv2608-a, or Rv2608-b. In some embodiments, the strong Mycobacterial central memory T cell activator antigen comprises a sequence having at least 90% sequence identity to Rv1813-b or Rv2608-b. In some embodiments, the strong Mycobacterial central memory T cell activator antigen comprises a sequence having at least 90% sequence identity to Rv1813-b. In some embodiments, the strong Mycobacterial central memory T cell activator antigen comprises a sequence having at least 90% sequence identity to Rv2608-b.

In some embodiments, the strong Mycobacterial central memory T cell activator antigen comprises the sequence of Rv1813-a, Rv1813-b, Rv1886-a, Rv1886-b, Rv2389-a, Rv2389-b, Rv2608-a, or Rv2608-b. In some embodiments, the strong Mycobacterial central memory T cell activator antigen comprises the sequence of Rv1813-b, Rv2389-b, Rv1886b, or Rv2608-b. In some embodiments, the strong Mycobacterial central memory T cell activator antigen comprises the sequence of Rv1813-a, Rv1813-b, Rv2608-a, or Rv2608-b. In some embodiments, the strong Mycobacterial central memory T cell activator antigen comprises the sequence of Rv1813-b or Rv2608-b. In some embodiments, the strong Mycobacterial central memory T cell activator antigen comprises the sequence of Rv1813-b. In some embodiments, the strong Mycobacterial central memory T cell activator antigen comprises the sequence of Rv2608-b.

In some embodiments, the strong Mycobacterial effector memory T cell activator antigen comprises a sequence having at least 90% sequence identity to Rv3619 or Rv3620. In some embodiments, the strong Mycobacterial effector memory T cell activator antigen comprises a sequence having at least 90% sequence identity to Rv3619. In some embodiments, the strong Mycobacterial effector memory T cell activator antigen comprises a sequence having at least 90% sequence identity to Rv3620.

In some embodiments, the strong Mycobacterial effector memory T cell activator antigen comprises the sequence of Rv3619 or Rv3620. In some embodiments, the strong Mycobacterial effector memory T cell activator antigen comprises the sequence of Rv3619. In some embodiments, the strong Mycobacterial effector memory T cell activator antigen comprises the sequence of Rv3620.

In some embodiments, the strong central memory T cell activator antigen comprises a sequence having at least 90% sequence identity to Rv1813-a, Rv1813-b, Rv2608-a, or Rv2608-b and the strong Mycobacterial effector memory T cell activator antigen comprises a sequence having at least 90% sequence identity to Rv3619 or Rv3620.

In some embodiments, the strong central memory T cell activator antigen comprises the sequence of Rv1813-a, Rv1813-b, Rv2608-a, or Rv2608-b and the strong Mycobacterial effector memory T cell activator antigen comprises the sequence of Rv3619 or Rv3620.

In some embodiments, the strong central memory T cell activator antigen comprises a sequence having at least 90% sequence identity to Rv1813-a, Rv1813-b, Rv2608-a, or Rv2608-b and the strong Mycobacterial effector memory T cell activator antigen comprises a sequence having at least 90% sequence identity to Rv3619. In some embodiments, the strong central memory T cell activator antigen comprises a sequence having at least 90% sequence identity to Rv1813-a, Rv1813-b, Rv2608-a, or Rv2608-b and the strong Mycobacterial effector memory T cell activator antigen comprises a sequence having at least 90% sequence identity to Rv3620.

In some embodiments, the strong central memory T cell activator antigen comprises the sequence of Rv1813-a, Rv1813-b, Rv2608-a, or Rv2608-b and the strong Mycobacterial effector memory T cell activator antigen comprises the sequence of Rv3619. In some embodiments, the strong central memory T cell activator antigen comprises the sequence of Rv1813-a, Rv1813-b, Rv2608-a, or Rv2608-b and the strong Mycobacterial effector memory T cell activator antigen comprises the sequence of Rv3620.

In some embodiments, the strong central memory T cell activator antigen comprises a sequence having at least 90% sequence identity to Rv1813-b or Rv2608-b and the strong Mycobacterial effector memory T cell activator antigen comprises a sequence having at least 90% sequence identity to Rv3619. In some embodiments, the strong central memory T cell activator antigen comprises a sequence having at least 90% sequence identity to Rv1813-b or Rv2608-b and the strong Mycobacterial effector memory T cell activator antigen comprises a sequence having at least 90% sequence identity to Rv3620.

In some embodiments, the strong central memory T cell activator antigen comprises the sequence of Rv1813-b or Rv2608-b and the strong Mycobacterial effector memory T cell activator antigen comprises the sequence of Rv3619. In some embodiments, the strong central memory T cell activator antigen comprises the sequence of Rv1813-b or Rv2608-b and the strong Mycobacterial effector memory T cell activator antigen comprises the sequence of Rv3620.

In some of the fusion polypeptides provided herein, the strong Mycobacterial central memory T cell activator antigen is a nontuberculous Mycobacterial (NTM) antigen.

In some of the fusion polypeptides provided herein, the strong Mycobacterial central memory T cell activator antigen is a Mycobacterium tuberculosis (Mtb) antigen.

In some of the fusion polypeptides provided herein, the strong Mycobacterial effector memory T cell activator antigen is a NTM antigen.

In some of the fusion polypeptides provided herein, the strong Mycobacterial effector memory T cell activator antigen is an Mtb antigen.

In some of the fusion polypeptides provided herein, the strong Mycobacterial central memory T cell activator antigen is a Mtb antigen and the strong Mycobacterial effector memory T cell activator antigen is a Mtb antigen.

In some of the fusion polypeptides provided herein, the strong Mycobacterial central memory T cell activator antigen is a NTM antigen and the strong Mycobacterial effector memory T cell activator antigen is an Mtb antigen.

In some of the fusion polypeptides provided herein, the strong Mycobacterial central memory T cell activator antigen is a Mtb antigen and the strong Mycobacterial effector memory T cell activator antigen is an NTM antigen.

In some of the fusion polypeptides provided herein, the strong Mycobacterial central memory T cell activator antigen is a NTM antigen and the strong Mycobacterial effector memory T cell activator antigen is a NTM antigen.

In some embodiments, the fusion polypeptide comprises antigens having at least 90% sequence identity to Rv3619, Rv3620, Rv2389-b, and Rv2608-b.

In some embodiments, the fusion polypeptide Rv3619, Rv3620, Rv2389-b, and Rv2608-b.

In some embodiments, the fusion polypeptide has at least 90% sequence identity to sequence of any of the fusion polypeptides provided in Table 2. In some embodiments, the fusion polypeptide is any one of the fusion polypeptides provided in Table 2.

In some embodiments, the fusion polypeptide has at least 90% sequence identity to ID93-1 or ID93-2. In some embodiments, the fusion polypeptide is ID93-1 or ID93-2.

In some embodiments, the fusion polypeptide has at least 90% sequence identity to ID93-1 or ID93-2. In some embodiments, the fusion polypeptide is ID93-1 or ID93-2.

In some embodiments, the fusion polypeptide has at least 90% sequence identity to ID93-1 or ID93-2. In some embodiments, the fusion polypeptide is ID93-1 or ID93-2.

In some embodiments, the fusion polypeptide has at least 90% sequence identity to ID83-1 or ID83-2. In some embodiments, the fusion polypeptide is ID83-1 or ID83-2.

In some embodiments, the fusion polypeptide has at least 90% sequence identity to ID97. In some embodiments, the fusion polypeptide is ID97.

TABLE 1
Exemplary Antigens

| | |
|---|---|
| Rv0496-a | Rv0496-a |
| | VVDAHRGGHPTPMSSTKATLRLAEATDSSGKITKRGADKLISTIDEFAKIA |
| | ISSGCAELMAFATSAVRDAENSEDVLSRVRKETGVELQALRGEDESRLTF |
| | LAVRRWYGWSAGRILNLDIGGGSLEVSSGVDEEPEIALSLPLGAGRLTRE |
| | WLPDDPPGRRRVAMLRDWLDAELAEPSVTVLEAGSPDLAVATSKTFRSL |
| | ARLTGAAPSMAGPRVKRTLTANGLRQLIAFISRMTAVDRAELEGVSADR |
| | APQIVAGALVAEASMRALSIEAVEICPWALREGLILRKLDSEADGTALIES |
| | SSVHTSVRAVGGQPADRNAANRSRGSKP |
| Rv0496-b | Rv0496-b |
| | VDAHRGGHPTPMSSTKATLRLAEATDSSGKITKRGADKLISTIDEFAKIAI |
| | SSGCAELMAFATSAVRDAENSEDVLSRVRKETGVELQALRGEDESRLTFL |
| | AVRRWYGWSAGRILNLDIGGGSLEVSSGVDEEPEIALSLPLGAGRLTREW |
| | LPDDPPGRRRVAMLRDWLDAELAEPSVTVLEAGSPDLAVATSKTFRSLA |
| | RLTGAAPSMAGPRVKRTLTANGLRQLIAFISRMTAVDRAELEGVSADRA |
| | PQIVAGALVAEASMRALSIEAVEICPWALREGLILRKLDSEADGTALIESS |
| | SVHTSVRAVGGQPADRNAANRSRGSKP |
| Rv1813-a | Rv1813-a |
| | MITNLRRRTAMAAAGLGAALGLGILLVPTVDAHLANGSMSEVMMSEIA |
| | GLPIPPIIHYGAIAYAPSGASGKAWHQRTPARAEQVALEKCGDKTCKVVS |
| | RFTRCGAVAYNGSKYQGGTGLTRRAAEDDAVNRLEGGRIVNWACN |
| Rv1813-b | Rv1813-b |
| | HLANGSMSEVMMSEIAGLPIPPIIHYGAIAYAPSGASGKAWHQRTPARAE |
| | QVALEKCGDKTCKVVSRFTRCGAVAYNGSKYQGGTGLTRRAAEDDAV |
| | NRLEGGRIVNWACN |
| Rv1886-a | Rv1886-a |
| | MTDVSRKIRAWGRRLMIGTAAAVVLPGLVGLAGGAATAGAFSRPGLPV |
| | EYLQVPSPSMGRDIKVQFQSGGNNSPAVYLLDGLRAQDDYNGWDINTPA |
| | FEWYYQSGLSIVMPVGGQSSFYSDWYSPACGKAGCQTYKWETFLTSELP |
| | QWLSANRAVKPTGSAAIGLSMAGSSAMILAAYHPQQFIYAGSLSALLDPS |
| | QGMGPSLIGLAMGDAGGYKAADMWGPSSDPAWERNDPTQQIPKLVAN |
| | NTRLWVYCGNGTPNELGGANIPAEFLENFVRSSNLKFQDAYNAAGGHN |
| | AVFNFPPNGTHSWEYWGAQLNAMKGDLQSSLGAG |
| Rv1886-b | Rv1886-b |
| | FSRPGLPVEYLQVPSPSMGRDIKVQFQSGGNNSPAVYLLDGLRAQDDYN |
| | GWDINTPAFEWYYQSGLSIVMPVGGQSSFYSDWYSPACGKAGCQTYKW |
| | ETFLTSELPQWLSANRAVKPTGSAAIGLSMAGSSAMILAAYHPQQFIYAG |
| | SLSALLDPSQGMGPSLIGLAMGDAGGYKAADMWGPSSDPAWERNDPTQ |
| | QIPKLVANNTRLWVYCGNGTPNELGGANIPAEFLENFVRSSNLKFQDAY |
| | NAAGGHNAVFNEPPNGTHSWEYWGAQLNAMKGDLQSSLGAG |
| Rv2389-a | Rv2389-a |
| | MTPGLLTTAGAGRPRDRCARIVCTVFIETAVVATMFVALLGLSTISSKAD |
| | DIDWDAIAQCESGGNWAANTGNGLYGGLQISQATWDSNGGVGSPAAAS |
| | PQQQIEVADNIMKTQGPGAWPKCSSCSQGDAPLGSLTHILTFLAAETGGC |
| | SGSRDD |
| Rv2389-b | Rv2389-b |
| | DDIDWDAIAQCESGGNWAANTGNGLYGGLQISQATWDSNGGVGSPAAA |
| | SPQQQIEVADNIMKTQGPGAWPKCSSCSQGDAPLGSLTHILTFLAAETGG |
| | CSGSRDD |
| Rv2608-a | Rv2608-a |
| | MNFAVLPPEVNSARIFAGAGLGPMLAAASAWDGLAEELHAAAGSFASVT |
| | TGLAGDAWHGPASLAMTRAASPYVGWLNTAAGQAAQAAGQARLAASA |
| | FEATLAATVSPAMVAANRTRLASLVAANLLGQNAPAIAAAEAEYEQIWA |
| | QDVAAMFGYHSAASAVATQLAPIQEGLQQQLQNVLAQLASGNLGSGNV |
| | GVGNIGNDNIGNANIGFGNRGDANIGIGNIGDRNLGIGNTGNWNIGIGITG |
| | NGQIGFGKPANPDVLVVGNGGPGVTALVMGGTDSLLPLPNIPLLEYAARF |
| | ITPVHPGYTATFLETPSQFFPFTGLNSLTYDVSVAQGVTNLHTAIMAQLAA |
| | GNEVVVFGTSQSATIATFEMRYLQSLPAHLRPGLDELSFTLTGNPNRPDG |
| | GILTRFGFSIPQLGFTLSGATPADAYPTVDYAFQYDGVNDFPKYPLNVFAT |
| | ANAIAGILFLHSGLIALPPDLASGVVQPVSSPDVLTTYILLPSQDLPLLVPL |
| | RAIPLLGNPLADLIQPDLRVLVELGYDRTAHQDVPSPFGLFPDVDWAEVA |
| | ADLQQGAVQGVNDALSGLGLPPPWQPALPRLF |
| Rv2608-b | Rv2608-b |
| | NFAVLPPEVNSARIFAGAGLGPMLAAASAWDGLAEELHAAAGSFASVTT |
| | GLAGDAWHGPASLAMTRAASPYVGWLNTAAGQAAQAAGQARLAASAF |
| | EATLAATVSPAMVAANRTRLASLVAANLLGQNAPAIAAAEAEYEQIWA |
| | QDVAAMFGYHSAASAVATQLAPIQEGLQQQLQNVLAQLASGNLGSGNV |
| | GVGNIGNDNIGNANIGFGNRGDANIGIGNIGDRNLGIGNTGNWNIGIGITG |
| | NGQIGFGKPANPDVLVVGNGGPGVTALVMGGTDSLLPLPNIPLLEYAARF |
| | ITPVHPGYTATFLETPSQFFPFTGLNSLTYDVSVAQGVTNLHTAIMAQLAA |
| | GNEVVVFGTSQSATIATFEMRYLQSLPAHLRPGLDELSFTLTGNPNRPDG |
| | GILTRFGFSIPQLGFTLSGATPADAYPTVDYAFQYDGVNDFPKYPLNVFAT |
| | ANAIAGILFLHSGLIALPPDLASGVVQPVSSPDVLTTYILLPSQDLPLLVPL |
| | RAIPLLGNPLADLIQPDLRVLVELGYDRTAHQDVPSPFGLFPDVDWAEVA |
| | ADLQQGAVQGVNDALSGLGLPPPWQPALPRLF |
| Rv2875-a | Rv2875-a |
| | MKVKNTIAATSFAAAGLAALAVAVSPPAAAGDLVGPGCAEYAAANPTG |
| | PASVQGMSQDPVAVAASNNPELTTLTAALSGQLNPQVNLVDTLNSGQYT |
| | VFAPTNAAFSKLPASTIDELKTNSSLLTSILTYHVVAGQTSPANVVGTRQT |
| | LQGASVTVTGQGNSLKVGNADVVCGGVSTANATVYMIDSVLMPPA |
| Rv2875-b | Rv2875-b |
| | GDLVGPGCAEYAAANPTGPASVQGMSQDPVAVAASNNPELTTLTAALS |
| | GQLNPQVNLVDTLNSGQYTVFAPTNAAFSKLPASTIDELKTNSSLLTSILT |
| | YHVVAGQTSPANVVGTRQTLQGASVTVTGQGNSLKVGNADVVCGGVST |
| | ANATVYMIDSVLMPPA |
| Rv2875-c | Rv2875-c |
| | MKVKNTIAATSFAAAGLAALAVAVSPPAAAGDLVSPGCAEYAAANPTGP |
| | ASVQGMSQDPVAVAASNNPELTTLTAALSGQLNPQVNLVDTLNSGQYT |
| | VFAPTNAAFSKLPASTIDELKTNSSLLTSILTYHVVAGQTSPANVVGTRQT |
| | LQGASVTVTGQGNSLKVGNADVVCGGVSTANATVYMIDSVLMPPA |
| Rv2875-d | Rv2875-d |
| | GDLVSPGCAEYAAANPTGPASVQGMSQDPVAVAASNNPELTTLTAALSG |
| | QLNPQVNLVDTLNSGQYTVFAPTNAAFSKLPASTIDELKTNSSLLTSILTY |
| | HVVAGQTSPANVVGTRQTLQGASVTVTGQGNSLKVGNADVVCGGVSTA |
| | NATVYMIDSVLMPPA |
| Rv3478-a | Rv3478-a |
| | VVDFGALPPEINSARMYAGPGSASLVAAAKMWDSVASDLFSAASAFQSV |
| | VWGLTVGSWIGSSAGLMAAAASPYVAWMSVTAGQAQLTAAQVRVAAA |
| | AYETAYRLTVPPPVIAENRTELMTLTATNLLGQNTPAIEANQAAYSQMW |
| | GQDAEAMYGYAATAATATEALLPFEDAPLITNPGGLLEQAVAVEEAIDT |
| | AAANQLMNNVPQALQQLAQPAQGVVPSSKLGGLWTAVSPHLSPLSNVS |
| | SIANNHMSMMGTGVSMTNTLHSMLKGLAPAAAQAVETAAENGVWAMS |
| | SLGSQLGSSLGSSGLGAGVAANLGRAASVGSLSVPPAWAAANQAVTPAA |
| | RALPLTSLTSAAQTAPGHMLGGLPLGHSVNAGSGINNALRVPARAYAIPR |
| | TPAAG |
| Rv3478-b | Rv3478-b |
| | VVDFGALPPEINSARMYAGPGSASLVAAAKMWDSVASDLFSAASAFQSV |
| | VWGLTVGSWIGSSAGLMAAAASPYVAWMSVTAGQAQLTAAQVRVAAA |
| | AYETAYRLTVPPPVIAENRTELMTLTATNLLGQNTPAIEANQAAYSQMW |
| | GQDAEAMYGYAATAATATEALLPFEDAPLITNPGG |
| Rv3619 | Rv3619 |
| | MTINYQFGDVDAHGAMIRAQAGSLEAEHQAIISDVLTASDFWGGAGSAA |
| | CQGFITQLGRNFQVIYEQANAHGQKVQAAGNNMAQTDSAVGSSWA |
| Rv3620 | Rv3620 |
| | MTSRFMTDPHAMRDMAGRFEVHAQTVEDEARRMWASAQNISGAGWSG |
| | MAEATSLDTMTQMNQAFRNIVNMLHGVRDGLVRDANNYEQQEQASQQ |
| | ILSS |
| Rv3810-a | Rv3810-a |
| | VPNRRRRKLSTAMSAVAALAVASPCAYFLVYESTETTERPEHHEFKQAA |
| | VLTDLPGELMSALSQGLSQFGINIPPVPSLTGSGDASTGLTGPGLTSPGLTS |
| | PGLTSPGLTDPALTSPGLTPTLPGSLAAPGTTLAPTPGVGANPALTNPALT |
| | SPTGATPGLTSPTGLDPALGGANEIPITTPVGLDPGADGTYPILGDPTLGTI |
| | PSSPATTSTGGGGLVNDVMQVANELGASQAIDLLKGVLMPSINIQAVQNG |
| | GAAAPAASPPVPPIPAAAAVPPTDPITVPVA |
| Rv3810-b | Rv3810-b |
| | SPCAYFLVYESTETTERPEHHEFKQAAVLTDLPGELMSALSQGLSQFGINI |
| | PPVPSLTGSGDASTGLTGPGLTSPGLTSPGLTSPGLTDPALTSPGLTPTLPG |
| | SLAAPGTTLAPTPGVGANPALTNPALTSPTGATPGLTSPTGLDPALGGAN |
| | EIPITTPVGLDPGADGTYPILGDPTLGTIPSSPATTSTGGGGLVNDVMQVA |
| | NELGASQAIDLLKGVLMPSINIQAVQNGGAAAPAASPPVPPIPAAAAVPPT |
| | DPITVPVA |

TABLE 2
Exemplary Fusion Polypeptides

| | |
|---|---|
| ID58 | ID58 |
| | HLANGSMSEVMMSEIAGLPIPPIIHYGAIAYAPSGASGKAWHQRTPARAE |
| | QVALEKCGDKTCKVVSRFTRCGAVAYNGSKYQGGTGLTRRAAEDDAV |
| | NRLEGGRIVNWACNELMTSRFMTDPHAMRDMAGRFEVHAQTVEDEAR |
| | RMWASAQNISGAGWSGMAEATSLDTMTQMNQAFRNIVNMLHGVRDGL |
| | VRDANNYEQQEQASQQILSSVDVVDAHRGGHPTPMSSTKATLRLAEATD |
| | SSGKITKRGADKLISTIDEFAKIAISSGCAELMAFATSAVRDAENSEDVLSR |
| | VRKETGVELQALRGEDESRLTFLAVRRWYGWSAGRILNLDIGGGSLEVS |
| | SGVDEEPEIALSLPLGAGRLTREWLPDDPPGRRRVAMLRDWLDAELAEPS |
| | VTVLEAGSPDLAVATSKTFRSLARLTGAAPSMAGPRVKRTLTANGLRQLI |
| | AFISRMTAVDRAELEGVSADRAPQIVAGALVAEASMRALSIEAVEICPWA |
| | LREGLILRKLDSEADGTALIESSSVHTSVRAVGGQPADRNAANRSRGSKP |
| | ST |
| ID69 | ID69 |
| | DDIDWDAIAQCESGGNWAANTGNGLYGGLQISQATWDSNGGVGSPAAA |
| | SPQQQIEVADNIMKTQGPGAWPKCSSCSQGDAPLGSLTHILTFLAAETGG |
| | CSGSRDDGTHLANGSMSEVMMSEIAGLPIPPIIHYGAIAYAPSGASGKAW |
| | HQRTPARAEQVALEKCGDKTCKVVSRFTRCGAVAYNGSKYQGGTGLTR |
| | RAAEDDAVNRLEGGRIVNWACNELMTSRFMTDPHAMRDMAGRFEVHA |
| | QTVEDEARRMWASAQNISGAGWSGMAEATSLDTMTQMNQAFRNIVNM |
| | LHGVRDGLVRDANNYEQQEQASQQILSSVDMVDAHRGGHPTPMSSTKA |
| | TLRLAEATDSSGKITKRGADKLISTIDEFAKIAISSGCAELMAFATSAVRD |
| | AENSEDVLSRVRKETGVELQALRGEDESRLTFLAVRRWYGWSAGRILNL |
| | DIGGGSLEVSSGVDEEPEIALSLPLGAGRLTREWLPDDPPGRRRVAMLRD |
| | WLDAELAEPSVTVLEAGSPDLAVATSKTFRSLARLTGAAPSMAGPRVKR |
| | TLTANGLRQLIAFISRMTAVDRAELEGVSADRAPQIVAGALVAEASMRAL |
| | SIEAVEICPWALREGLILRKLDSEADGTALIESSSVHTSVRAVGGQPADRN |
| | AANRSRGSKPST |
| ID71 | ID71 |
| | HMMTINYQFGDVDAHGAMIRAQAGSLEAEHQAIISDVLTASDFWGGAGS |
| | AACQGFITQLGRNFQVIYEQANAHGQKVQAAGNNMAQTDSAVGSSWA |
| | GTDDIDWDAIAQCESGGNWAANTGNGLYGGLQISQATWDSNGGVGSPA |
| | AASPQQQIEVADNIMKTQGPGAWPKCSSCSQGDAPLGSLTHILTFLAAET |
| | GGCSGSRDDGSVVDFGALPPEINSARMYAGPGSASLVAAAKMWDSVAS |
| | DLFSAASAFQSVVWGLTVGSWIGSSAGLMAAAASPYVAWMSVTAGQA |
| | QLTAAQVRVAAAAYETAYRLTVPPPVIAENRTELMTLTATNLLGQNTPAI |
| | EANQAAYSQMWGQDAEAMYGYAATAATATEALLPFEDAPLITNPGGEF |
| | FSRPGLPVEYLQVPSPSMGRDIKVQFQSGGNNSPAVYLLDGLRAQDDYN |
| | GWDINTPAFEWYYQSGLSIVMPVGGQSSFYSDWYSPACGKAGCQTYKW |
| | ETFLTSELPQWLSANRAVKPTGSAAIGLSMAGSSAMILAAYHPQQFIYAG |
| | SLSALLDPSQGMGPSLIGLAMGDAGGYKAADMWGPSSDPAWERNDPTQ |
| | QIPKLVANNTRLWVYCGNGTPNELGGANIPAEFLENFVRSSNLKFQDAY |
| | NAAGGHNAVFNFPPNGTHSWEYWGAQLNAMKGDLQSSLGAG |
| ID83-1 | ID83-1 |
| | HLANGSMSEVMMSEIAGLPIPPIIHYGAIAYAPSGASGKAWHQRTPARAE |
| | QVALEKCGDKTCKVVSRFTRCGAVAYNGSKYQGGTGLTRRAAEDDAV |
| | NRLEGGRIVNWACNELMTSRFMTDPHAMRDMAGRFEVHAQTVEDEAR |
| | RMWASAQNISGAGWSGMAEATSLDTMTQMNQAFRNIVNMLHGVRDGL |
| | VRDANNYEQQEQASQQILSSVDINFAVLPPEVNSARIFAGAGLGPMLAAA |
| | SAWDGLAEELHAAAGSFASVTTGLAGDAWHGPASLAMTRAASPYVGW |
| | LNTAAGQAAQAAGQARLAASAFEATLAATVSPAMVAANRTRLASLVAA |
| | NLLGQNAPAIAAAEAEYEQIWAQDVAAMFGYHSAASAVATQLAPIQEGL |
| | QQQLQNVLAQLASGNLGSGNVGVGNIGNDNIGNANIGFGNRGDANIGIG |
| | NIGDRNLGIGNTGNWNIGIGITGNGQIGFGKPANPDVLVVGNGGPGVTAL |
| | VMGGTDSLLPLPNIPLLEYAARFITPVHPGYTATFLETPSQFFPFTGLNSLT |
| | YDVSVAQGVTNLHTAIMAQLAAGNEVVVFGTSQSATIATFEMRYLQSLP |
| | AHLRPGLDELSFTLTGNPNRPDGGILTRFGFSIPQLGFTLSGATPADAYPT |
| | VDYAFQYDGVNDFPKYPLNVFATANAIAGILFLHSGLIALPPDLASGVVQ |
| | PVSSPDVLTTYILLPSQDLPLLVPLRAIPLLGNPLADLIQPDLRVLVELGYD |
| | RTAHQDVPSPFGLFPDVDWAEVAADLQQGAVQGVNDALSGLGLPPPWQ |
| | PALPRLFST |
| ID83-2 | ID83-2 |
| | HLANGSMSEVMMSEIAGLPIPPIIHYGAIAYAPSGASGKAWHQRTPARAE |
| | QVALEKCGDKTCKVVSRFTRCGAVAYNGSKYQGGTGLTRRAAEDDAV |
| | NRLEGGRIVNWACNELMTSRFMTDPHAMRDMAGRFEVHAQTVEDEAR |
| | RMWASAQNISGAGWSGMAEATSLDTMTQMNQAFRNIVNMLHGVRDGL |
| | VRDANNYEQQEQASQQILSSVDMNFAVLPPEVNSARIFAGAGLGPMLAA |
| | ASAWDGLAEELHAAAGSFASVTTGLAGDAWHGPASLAMTRAASPYVG |
| | WLNTAAGQAAQAAGQARLAASAFEATLAATVSPAMVAANRTRLASLV |
| | AANLLGQNAPAIAAAEAEYEQIWAQDVAAMFGYHSAASAVATQLAPIQ |
| | EGLQQQLQNVLAQLASGNLGSGNVGVGNIGNDNIGNANIGFGNRGDANI |
| | GIGNIGDRNLGIGNTGNWNIGIGITGNGQIGFGKPANPDVLVVGNGGPGV |
| | TALVMGGTDSLLPLPNIPLLEYAARFITPVHPGYTATFLETPSQFFPFTGLN |
| | SLTYDVSVAQGVTNLHTAIMAQLAAGNEVVVFGTSQSATIATFEMRYLQ |
| | SLPAHLRPGLDELSFTLTGNPNRPDGGILTRFGFSIPQLGFTLSGATPADAY |
| | PTVDYAFQYDGVNDFPKYPLNVFATANAIAGILFLHSGLIALPPDLASGV |
| | VQPVSSPDVLTTYILLPSQDLPLLVPLRAIPLLGNPLADLIQPDLRVLVELG |
| | YDRTAHQDVPSPFGLFPDVDWAEVAADLQQGAVQGVNDALSGLGLPPP |
| | WQPALPRLFST |
| ID87 | ID87 |
| | MGDLVSPGCAEYAAANPTGPASVQGMSQDPVAVAASNNPELTTLTAAL |
| | SGQLNPQVNLVDTLNSGQYTVFAPTNAAFSKLPASTIDELKTNSSLLTSIL |
| | TYHVVAGQTSPANVVGTRQTLQGASVTVTGQGNSLKVGNADVVCGGVS |
| | TANATVYMIDSVLMPPAGSVVDFGALPPEINSARMYAGPGSASLVAAAK |
| | MWDSVASDLFSAASAFQSVVWGLTVGSWIGSSAGLMAAAASPYVAWM |
| | SVTAGQAQLTAAQVRVAAAAYETAYRLTVPPPVIAENRTELMTLTATNL |
| | LGQNTPAIEANQAAYSQMWGQDAEAMYGYAATAATATEALLPFEDAPL |
| | ITNPGGLLEQAVAVEEAIDTAAANQLMNNVPQALQQLAQPAQGVVPSSK |
| | LGGLWTAVSPHLSPLSNVSSIANNHMSMMGTGVSMTNTLHSMLKGLAP |
| | AAAQAVETAAENGVWAMSSLGSQLGSSLGSSGLGAGVAANLGRAASVG |
| | SLSVPPAWAAANQAVTPAARALPLTSLTSAAQTAPGHMLGGLPLGHSVN |
| | AGSGINNALRVPARAYAIPRTPAAGEFFSRPGLPVEYLQVPSPSMGRDIKV |
| | QFQSGGNNSPAVYLLDGLRAQDDYNGWDINTPAFEWYYQSGLSIVMPV |
| | GGQSSFYSDWYSPACGKAGCQTYKWETFLTSELPQWLSANRAVKPTGS |
| | AAIGLSMAGSSAMILAAYHPQQFIYAGSLSALLDPSQGMGPSLIGLAMGD |
| | AGGYKAADMWGPSSDPAWERNDPTQQIPKLVANNTRLWVYCGNGTPN |
| | ELGGANIPAEFLENFVRSSNLKFQDAYNAAGGHNAVFNFPPNGTHSWEY |
| | WGAQLNAMKGDLQSSLGAG |
| ID91 | ID91 |
| | MTINYQFGDVDAHGAMIRAQAGSLEAEHQAIISDVLTASDFWGGAGSAA |
| | CQGFITQLGRNFQVIYEQANAHGQKVQAAGNNMAQTDSAVGSSWAGTD |
| | DIDWDAIAQCESGGNWAANTGNGLYGGLQISQATWDSNGGVGSPAAAS |
| | PQQQIEVADNIMKTQGPGAWPKCSSCSQGDAPLGSLTHILTFLAAETGGC |
| | SGSRDDGSVVDFGALPPEINSARMYAGPGSASLVAAAKMWDSVASDLFS |
| | AASAFQSVVWGLTVGSWIGSSAGLMAAAASPYVAWMSVTAGQAQLTA |
| | AQVRVAAAAYETAYRLTVPPPVIAENRTELMTLTATNLLGQNTPAIEAN |
| | QAAYSQMWGQDAEAMYGYAATAATATEALLPFEDAPLITNPGGLLEQA |
| | VAVEEAIDTAAANQLMNNVPQALQQLAQPAQGVVPSSKLGGLWTAVSP |
| | HLSPLSNVSSIANNHMSMMGTGVSMTNTLHSMLKGLAPAAAQAVETAA |
| | ENGVWAMSSLGSQLGSSLGSSGLGAGVAANLGRAASVGSLSVPPAWAA |
| | ANQAVTPAARALPLTSLTSAAQTAPGHMLGGLPLGHSVNAGSGINNALR |
| | VPARAYAIPRTPAAGEFFSRPGLPVEYLQVPSPSMGRDIKVQFQSGGNNSP |
| | AVYLLDGLRAQDDYNGWDINTPAFEWYYQSGLSIVMPVGGQSSFYSDW |
| | YSPACGKAGCQTYKWETFLTSELPQWLSANRAVKPTGSAAIGLSMAGSS |
| | AMILAAYHPQQFIYAGSLSALLDPSQGMGPSLIGLAMGDAGGYKAADM |
| | WGPSSDPAWERNDPTQQIPKLVANNTRLWVYCGNGTPNELGGANIPAEF |
| | LENFVRSSNLKFQDAYNAAGGHNAVFNFPPNGTHSWEYWGAQLNAMK |
| | GDLQSSLGAG |
| ID93-1 | ID93-1 |
| | MTINYQFGDVDAHGAMIRAQAGSLEAEHQAIISDVLTASDFWGGAGSAA |
| | CQGFITQLGRNFQVIYEQANAHGQKVQAAGNNMAQTDSAVGSSWAGTH |
| | LANGSMSEVMMSEIAGLPIPPIIHYGAIAYAPSGASGKAWHQRTPARAEQ |
| | VALEKCGDKTCKVVSRFTRCGAVAYNGSKYQGGTGLTRRAAEDDAVNR |
| | LEGGRIVNWACNELMTSRFMTDPHAMRDMAGRFEVHAQTVEDEARRM |
| | WASAQNISGAGWSGMAEATSLDTMTQMNQAFRNIVNMLHGVRDGLVR |
| | DANNYEQQEQASQQILSSVDINFAVLPPEVNSARIFAGAGLGPMLAAASA |
| | WDGLAEELHAAAGSFASVTTGLAGDAWHGPASLAMTRAASPYVGWLN |
| | TAAGQAAQAAGQARLAASAFEATLAATVSPAMVAANRTRLASLVAANL |
| | LGQNAPAIAAAEAEYEQIWAQDVAAMFGYHSAASAVATQLAPIQEGLQ |
| | QQLQNVLAQLASGNLGSGNVGVGNIGNDNIGNANIGFGNRGDANIGIGNI |
| | GDRNLGIGNTGNWNIGIGITGNGQIGFGKPANPDVLVVGNGGPGVTALV |
| | MGGTDSLLPLPNIPLLEYAARFITPVHPGYTATFLETPSQFFPFTGLNSLTY |
| | DVSVAQGVTNLHTAIMAQLAAGNEVVVFGTSQSATIATFEMRYLQSLPA |
| | HLRPGLDELSFTLTGNPNRPDGGILTRFGFSIPQLGFTLSGATPADAYPTV |
| | DYAFQYDGVNDFPKYPLNVFATANAIAGILFLHSGLIALPPDLASGVVQP |
| | VSSPDVLTTYILLPSQDLPLLVPLRAIPLLGNPLADLIQPDLRVLVELGYDR |
| | TAHQDVPSPFGLFPDVDWAEVAADLQQGAVQGVNDALSGLGLPPPWQP |
| | ALPRLFST |
| ID93-2 | ID93-2 |
| | MTINYQFGDVDAHGAMIRAQAGSLEAEHQAIISDVLTASDFWGGAGSAA |
| | CQGFITQLGRNFQVIYEQANAHGQKVQAAGNNMAQTDSAVGSSWAGTH |
| | LANGSMSEVMMSEIAGLPIPPIIHYGAIAYAPSGASGKAWHQRTPARAEQ |
| | VALEKCGDKTCKVVSRFTRCGAVAYNGSKYQGGTGLTRRAAEDDAVNR |
| | LEGGRIVNWACNELMTSRFMTDPHAMRDMAGRFEVHAQTVEDEARRM |
| | WASAQNISGAGWSGMAEATSLDTMTQMNQAFRNIVNMLHGVRDGLVR |
| | DANNYEQQEQASQQILSSVDMNFAVLPPEVNSARIFAGAGLGPMLAAAS |
| | AWDGLAEELHAAAGSFASVTTGLAGDAWHGPASLAMTRAASPYVGWL |
| | NTAAGQAAQAAGQARLAASAFEATLAATVSPAMVAANRTRLASLVAAN |
| | LLGQNAPAIAAAEAEYEQIWAQDVAAMFGYHSAASAVATQLAPIQEGLQ |
| | QQLQNVLAQLASGNLGSGNVGVGNIGNDNIGNANIGFGNRGDANIGIGNI |
| | GDRNLGIGNTGNWNIGIGITGNGQIGFGKPANPDVLVVGNGGPGVTALV |
| | MGGTDSLLPLPNIPLLEYAARFITPVHPGYTATFLETPSQFFPFTGLNSLTY |
| | DVSVAQGVTNLHTAIMAQLAAGNEVVVFGTSQSATIATFEMRYLQSLPA |
| | HLRPGLDELSFTLTGNPNRPDGGILTRFGFSIPQLGFTLSGATPADAYPTV |
| | DYAFQYDGVNDFPKYPLNVFATANAIAGILFLHSGLIALPPDLASGVVQP |
| | VSSPDVLTTYILLPSQDLPLLVPLRAIPLLGNPLADLIQPDLRVLVELGYDR |
| | TAHQDVPSPFGLFPDVDWAEVAADLQQGAVQGVNDALSGLGLPPPWQP |
| | ALPRLFST |
| ID94-1 | ID94-1 |
| | DDIDWDAIAQCESGGNWAANTGNGLYGGLQISQATWDSNGGVGSPAAA |
| | SPQQQIEVADNIMKTQGPGAWPKCSSCSQGDAPLGSLTHILTFLAAETGG |
| | CSGSRDDGTHLANGSMSEVMMSEIAGLPIPPIIHYGAIAYAPSGASGKAW |
| | HQRTPARAEQVALEKCGDKTCKVVSRFTRCGAVAYNGSKYQGGTGLTR |
| | RAAEDDAVNRLEGGRIVNWACNELMTSRFMTDPHAMRDMAGRFEVHA |
| | QTVEDEARRMWASAQNISGAGWSGMAEATSLDTMTQMNQAFRNIVNM |
| | LHGVRDGLVRDANNYEQQEQASQQILSSVDINFAVLPPEVNSARIFAGAG |
| | LGPMLAAASAWDGLAEELHAAAGSFASVTTGLAGDAWHGPASLAMTR |
| | AASPYVGWLNTAAGQAAQAAGQARLAASAFEATLAATVSPAMVAANR |
| | TRLASLVAANLLGQNAPAIAAAEAEYEQIWAQDVAAMFGYHSAASAVA |
| | TQLAPIQEGLQQQLQNVLAQLASGNLGSGNVGVGNIGNDNIGNANIGFG |
| | NRGDANIGIGNIGDRNLGIGNTGNWNIGIGITGNGQIGFGKPANPDVLVV |
| | GNGGPGVTALVMGGTDSLLPLPNIPLLEYAARFITPVHPGYTATFLETPSQ |
| | FFPFTGLNSLTYDVSVAQGVTNLHTAIMAQLAAGNEVVVFGTSQSATIAT |
| | FEMRYLQSLPAHLRPGLDELSFTLTGNPNRPDGGILTRFGFSIPQLGFTLSG |
| | ATPADAYPTVDYAFQYDGVNDFPKYPLNVFATANAIAGILFLHSGLIALP |
| | PDLASGVVQPVSSPDVLTTYILLPSQDLPLLVPLRAIPLLGNPLADLIQPDL |
| | RVLVELGYDRTAHQDVPSPFGLFPDVDWAEVAADLQQGAVQGVNDALS |
| | GLGLPPPWQPALPRLFST |
| ID94-2 | ID94-2 |
| | DDIDWDAIAQCESGGNWAANTGNGLYGGLQISQATWDSNGGVGSPAAA |
| | SPQQQIEVADNIMKTQGPGAWPKCSSCSQGDAPLGSLTHILTFLAAETGG |
| | CSGSRDDGTHLANGSMSEVMMSEIAGLPIPPIIHYGAIAYAPSGASGKAW |
| | HQRTPARAEQVALEKCGDKTCKVVSRFTRCGAVAYNGSKYQGGTGLTR |
| | RAAEDDAVNRLEGGRIVNWACNELMTSRFMTDPHAMRDMAGRFEVHA |
| | QTVEDEARRMWASAQNISGAGWSGMAEATSLDTMTQMNQAFRNIVNM |
| | LHGVRDGLVRDANNYEQQEQASQQILSSVDMNFAVLPPEVNSARIFAGA |
| | GLGPMLAAASAWDGLAEELHAAAGSFASVTTGLAGDAWHGPASLAMT |
| | RAASPYVGWLNTAAGQAAQAAGQARLAASAFEATLAATVSPAMVAAN |
| | RTRLASLVAANLLGQNAPAIAAAEAEYEQIWAQDVAAMFGYHSAASAV |
| | ATQLAPIQEGLQQQLQNVLAQLASGNLGSGNVGVGNIGNDNIGNANIGF |
| | GNRGDANIGIGNIGDRNLGIGNTGNWNIGIGITGNGQIGFGKPANPDVLV |
| | VGNGGPGVTALVMGGTDSLLPLPNIPLLEYAARFITPVHPGYTATFLETPS |
| | QFFPFTGLNSLTYDVSVAQGVTNLHTAIMAQLAAGNEVVVFGTSQSATIA |
| | TFEMRYLQSLPAHLRPGLDELSFTLTGNPNRPDGGILTRFGFSIPQLGFTLS |
| | GATPADAYPTVDYAFQYDGVNDFPKYPLNVFATANAIAGILFLHSGLIAL |
| | PPDLASGVVQPVSSPDVLTTYILLPSQDLPLLVPLRAIPLLGNPLADLIQPD |
| | LRVLVELGYDRTAHQDVPSPFGLFPDVDWAEVAADLQQGAVQGVNDAL |
| | SGLGLPPPWQPALPRLFST |
| ID95 | ID95 |
| | DDIDWDAIAQCESGGNWAANTGNGLYGGLQISQATWDSNGGVGSPAAA |
| | SPQQQIEVADNIMKTQGPGAWPKCSSCSQGDAPLGSLTHILTFLAAETGG |
| | CSGSRDDELSPCAYFLVYESTETTERPEHHEFKQAAVLTDLPGELMSALS |
| | QGLSQFGINIPPVPSLTGSGDASTGLTGPGLTSPGLTSPGLTSPGLTDPALT |
| | SPGLTPTLPGSLAAPGTTLAPTPGVGANPALTNPALTSPTGATPGLTSPTG |
| | LDPALGGANEIPITTPVGLDPGADGTYPILGDPTLGTIPSSPATTSTGGGGL |
| | VNDVMQVANELGASQAIDLLKGVLMPSIIVIQAVQNGGAAAPAASPPVPPI |
| | PAAAAVPPTDPITVPVAGTHLANGSMSEVMMSEIAGLPIPPIIHYGAIAYA |
| | PSGASGKAWHQRTPARAEQVALEKCGDKTCKVVSRFTRCGAVAYNGSK |
| | YQGGTGLTRRAAEDDAVNRLEGGRIVNWACNELMTSRFMTDPHAMRD |
| | MAGRFEVHAQTVEDEARRMWASAQNISGAGWSGMAEATSLDTMTQMN |
| | QAFRNIVNMLHGVRDGLVRDANNYEQQEQASQQILSSVDMVDAHRGGH |
| | PTPMSSTKATLRLAEATDSSGKITKRGADKLISTIDEFAKIAISSGCAELMA |
| | FATSAVRDAENSEDVLSRVRKETGVELQALRGEDESRLTFLAVRRWYG |
| | WSAGRILNLDIGGGSLEVSSGVDEEPEIALSLPLGAGRLTREWLPDDPPGR |
| | RRVAMLRDWLDAELAEPSVTVLEAGSPDLAVATSKTFRSLARLTGAAPS |
| | MAGPRVKRTLTANGLRQLIAFISRMTAVDRAELEGVSADRAPQIVAGAL |
| | VAEASMRALSIEAVEICPWALREGLILRKLDSEADGTALIESSSVHTSVRA |
| | VGGQPADRNAANRSRGSKPST |
| ID97 | ID97 |
| | MTINYQFGDVDAHGAMIRAQAGSLEAEHQAIISDVLTASDFWGGAGSAA |
| | CQGFITQLGRNFQVIYEQANAHGQKVQAAGNNMAQTDSAVGSSWAGT |
| | MGDLVSPGCAEYAAANPTGPASVQGMSQDPVAVAASNNPELTTLTAAL |
| | SGQLNPQVNLVDTLNSGQYTVFAPTNAAFSKLPASTIDELKTNSSLLTSIL |
| | TYHVVAGQTSPANVVGTRQTLQGASVTVTGQGNSLKVGNADVVCGGVS |
| | TANATVYMIDSVLMPPAGSVVDFGALPPEINSARMYAGPGSASLVAAAK |
| | MWDSVASDLFSAASAFQSVVWGLTVGSWIGSSAGLMAAAASPYVAWM |
| | SVTAGQAQLTAAQVRVAAAAYETAYRLTVPPPVIAENRTELMTLTATNL |
| | LGQNTPAIEANQAAYSQMWGQDAEAMYGYAATAATATEALLPFEDAPL |
| | ITNPGGLLEQAVAVEEAIDTAAANQLMNNVPQALQQLAQPAQGVVPSSK |
| | LGGLWTAVSPHLSPLSNVSSIANNHMSMMGTGVSMTNTLHSMLKGLAP |
| | AAAQAVETAAENGVWAMSSLGSQLGSSLGSSGLGAGVAANLGRAASVG |
| | SLSVPPAWAAANQAVTPAARALPLTSLTSAAQTAPGHMLGGLPLGHSVN |
| | AGSGINNALRVPARAYAIPRTPAAGEFFSRPGLPVEYLQVPSPSMGRDIKV |
| | QFQSGGNNSPAVYLLDGLRAQDDYNGWDINTPAFEWYYQSGLSIVMPV |
| | GGQSSFYSDWYSPACGKAGCQTYKWETFLTSELPQWLSANRAVKPTGS |
| | AAIGLSMAGSSAMILAAYHPQQFIYAGSLSALLDPSQGMGPSLIGLAMGD |
| | AGGYKAADMWGPSSDPAWERNDPTQQIPKLVANNTRLWVYCGNGTPN |
| | ELGGANIPAEFLENFVRSSNLKFQDAYNAAGGHNAVFNFPPNGTHSWEY |
| | WGAQLNAMKGDLQSSLGAG |
| ID114 | ID114 |
| | GTHLANGSMSEVMMSEIAGLPIPPIIHYGAIAYAPSGASGKAWHQRTPAR |
| | AEQVALEKCGDKTCKVVSRFTRCGAVAYNGSKYQGGTGLTRRAAEDDA |
| | VNRLEGGRIVNWACNELMTSRFMTDPHAMRDMAGRFEVHAQTVEDEA |
| | RRMWASAQNISGAGWSGMAEATSLDTMTQMNQAFRNIVNMLHGVRDG |
| | LVRDANNYEQQEQASQQILSSVDMNFAVLPPEVNSARIFAGAGLGPMLA |
| | AASAWDGLAEELHAAAGSFASVTTGLAGDAWHGPASLAMTRAASPYV |
| | GWLNTAAGQAAQAAGQARLAASAFEATLAATVSPAMVAANRTRLASL |
| | VAANLLGQNAPAIAAAEAEYEQIWAQDVAAMFGYHSAASAVATQLAPI |
| | QEGLQQQLQNVLAQLASGNLGSGNVGVGNIGNDNIGNANIGFGNRGDA |
| | NIGIGNIGDRNLGIGNTGNWNIGIGITGNGQIGFGKPANPDVLVVGNGGPG |
| | VTALVMGGTDSLLPLPNIPLLEYAARFITPVHPGYTATFLETPSQFFPFTGL |
| | NSLTYDVSVAQGVTNLHTAIMAQLAAGNEVVVFGTSQSATIATFEMRYL |
| | QSLPAHLRPGLDELSFTLTGNPNRPDGGILTRFGFSIPQLGFTLSGATPADA |
| | YPTVDYAFQYDGVNDFPKYPLNVFATANAIAGILFLHSGLIALPPDLASG |
| | VVQPVSSPDVLTTYILLPSQDLPLLVPLRAIPLLGNPLADLIQPDLRVLVEL |
| | GYDRTAHQDVPSPFGLFPDVDWAEVAADLQQGAVQGVNDALSGLGLPP |
| | PWQPALPRLFSTFSRPGLPVEYLQVPSPSMGRDIKVQFQSGGNNSPAVYL |
| | LDGLRAQDDYNGWDINTPAFEWYYQSGLSIVMPVGGQSSFYSDWYSPA |
| | CGKAGCQTYKWETFLTSELPQWLSANRAVKPTGSAAIGLSMAGSSAMIL |
| | AAYHPQQFIYAGSLSALLDPSQGMGPSLIGLAMGDAGGYKAADMWGPS |
| | SDPAWERNDPTQQIPKLVANNTRLWVYCGNGTPNELGGANIPAEFLENF |
| | VRSSNLKFQDAYNAAGGHNAVFNFPPNGTHSWEYWGAQLNAMKGDLQ |
| | SSLGAG |
| ID120-1 | ID120-1 |
| | DDIDWDAIAQCESGGNWAANTGNGLYGGLQISQATWDSNGGVGSPAAA |
| | SPQQQIEVADNIMKTQGPGAWPKCSSCSQGDAPLGSLTHILTFLAAETGG |
| | CSGSRDDELSPCAYFLVYESTETTERPEHHEFKQAAVLTDLPGELMSALS |
| | QGLSQFGINIPPVPSLTGSGDASTGLTGPGLTSPGLTSPGLTSPGLTDPALT |
| | SPGLTPTLPGSLAAPGTTLAPTPGVGANPALTNPALTSPTGATPGLTSPTG |
| | LDPALGGANEIPITTPVGLDPGADGTYPILGDPTLGTIPSSPATTSTGGGGL |
| | VNDVMQVANELGASQAIDLLKGVLMPSINIQAVQNGGAAAPAASPPVPPI |
| | PAAAAVPPTDPITVPVAGTHLANGSMSEVMMSEIAGLPIPPIIHYGAIAYA |
| | PSGASGKAWHQRTPARAEQVALEKCGDKTCKVVSRFTRCGAVAYNGSK |
| | YQGGTGLTRRAAEDDAVNRLEGGRIVNWACNELMTSRFMTDPHAMRD |
| | MAGRFEVHAQTVEDEARRMWASAQNISGAGWSGMAEATSLDTMTQMN |
| | QAFRNIVNMLHGVRDGLVRDANNYEQQEQASQQILSSVDINFAVLPPEV |
| | NSARIFAGAGLGPMLAAASAWDGLAEELHAAAGSFASVTTGLAGDAWH |
| | GPASLAMTRAASPYVGWLNTAAGQAAQAAGQARLAASAFEATLAATVS |
| | PAMVAANRTRLASLVAANLLGQNAPAIAAAEAEYEQIWAQDVAAMFGY |
| | HSAASAVATQLAPIQEGLQQQLQNVLAQLASGNLGSGNVGVGNIGNDNI |
| | GNANIGFGNRGDANIGIGNIGDRNLGIGNTGNWNIGIGITGNGQIGFGKPA |
| | NPDVLVVGNGGPGVTALVMGGTDSLLPLPNIPLLEYAARFITPVHPGYTA |
| | TFLETPSQFFPFTGLNSLTYDVSVAQGVTNLHTAIMAQLAAGNEVVVFGT |
| | SQSATIATFEMRYLQSLPAHLRPGLDELSFTLTGNPNRPDGGILTRFGFSIP |
| | QLGFTLSGATPADAYPTVDYAFQYDGVNDFPKYPLNVFATANAIAGILFL |
| | HSGLIALPPDLASGVVQPVSSPDVLTTYILLPSQDLPLLVPLRAIPLLGNPL |
| | ADLIQPDLRVLVELGYDRTAHQDVPSPFGLFPDVDWAEVAADLQQGAV |
| | QGVNDALSGLGLPPPWQPALPRLFST |
| ID120-2 | ID120-2 |
| | DDIDWDAIAQCESGGNWAANTGNGLYGGLQISQATWDSNGGVGSPAAA |
| | SPQQQIEVADNIMKTQGPGAWPKCSSCSQGDAPLGSLTHILTFLAAETGG |
| | CSGSRDDELSPCAYFLVYESTETTERPEHHEFKQAAVLTDLPGELMSALS |
| | QGLSQFGINIPPVPSLTGSGDASTGLTGPGLTSPGLTSPGLTSPGLTDPALT |
| | SPGLTPTLPGSLAAPGTTLAPTPGVGANPALTNPALTSPTGATPGLTSPTG |
| | LDPALGGANEIPITTPVGLDPGADGTYPILGDPTLGTIPSSPATTSTGGGGL |
| | VNDVMQVANELGASQAIDLLKGVLMPSIIVIQAVQNGGAAAPAASPPVPPI |
| | PAAAAVPPTDPITVPVAGTHLANGSMSEVMMSEIAGLPIPPIIHYGAIAYA |
| | PSGASGKAWHQRTPARAEQVALEKCGDKTCKVVSRFTRCGAVAYNGSK |
| | YQGGTGLTRRAAEDDAVNRLEGGRIVNWACNELMTSRFMTDPHAMRD |
| | MAGRFEVHAQTVEDEARRMWASAQNISGAGWSGMAEATSLDTMTQMN |
| | QAFRNIVNMLHGVRDGLVRDANNYEQQEQASQQILSSVDMNFAVLPPEV |
| | NSARIFAGAGLGPMLAAASAWDGLAEELHAAAGSFASVTTGLAGDAWH |
| | GPASLAMTRAASPYVGWLNTAAGQAAQAAGQARLAASAFEATLAATVS |
| | PAMVAANRTRLASLVAANLLGQNAPAIAAAEAEYEQIWAQDVAAMFGY |
| | HSAASAVATQLAPIQEGLQQQLQNVLAQLASGNLGSGNVGVGNIGNDNI |
| | GNANIGFGNRGDANIGIGNIGDRNLGIGNTGNWNIGIGITGNGQIGFGKPA |
| | NPDVLVVGNGGPGVTALVMGGTDSLLPLPNIPLLEYAARFITPVHPGYTA |
| | TFLETPSQFFPFTGLNSLTYDVSVAQGVTNLHTAIMAQLAAGNEVVVFGT |
| | SQSATIATFEMRYLQSLPAHLRPGLDELSFTLTGNPNRPDGGILTRFGFSIP |
| | QLGFTLSGATPADAYPTVDYAFQYDGVNDFPKYPLNVFATANAIAGILFL |
| | HSGLIALPPDLASGVVQPVSSPDVLTTYILLPSQDLPLLVPLRAIPLLGNPL |
| | ADLIQPDLRVLVELGYDRTAHQDVPSPFGLFPDVDWAEVAADLQQGAV |
| | QGVNDALSGLGLPPPWQPALPRLFST |
| ID125-1 | ID125-1 |
| | MTINYQFGDVDAHGAMIRAQAGSLEAEHQAIISDVLTASDFWGGAGSAA |
| | CQGFITQLGRNFQVIYEQANAHGQKVQAAGNNMAQTDSAVGSSWAGTH |
| | LANGSMSEVMMSEIAGLPIPPIIHYGAIAYAPSGASGKAWHQRTPARAEQ |
| | VALEKCGDKTCKVVSRFTRCGAVAYNGSKYQGGTGLTRRAAEDDAVNR |
| | LEGGRIVNWACNELMTSRFMTDPHAMRDMAGRFEVHAQTVEDEARRM |
| | WASAQNISGAGWSGMAEATSLDTMTQMNQAFRNIVNMLHGVRDGLVR |
| | DANNYEQQEQASQQILSSVDINFAVLPPEVNSARIFAGAGLGPMLAAASA |
| | WDGLAEELHAAAGSFASVTTGLAGDAWHGPASLAMTRAASPYVGWLN |
| | TAAGQAAQAAGQARLAASAFEATLAATVSPAMVAANRTRLASLVAANL |
| | LGQNAPAIAAAEAEYEQIWAQDVAAMFGYHSAASAVATQLAPIQEGLQ |
| | QQLQNVLAQLASGNLGSGNVGVGNIGNDNIGNANIGFGNRGDANIGIGNI |
| | GDRNLGIGNTGNWNIGIGITGNGQIGFGKPANPDVLVVGNGGPGVTALV |
| | MGGTDSLLPLPNIPLLEYAARFITPVHPGYTATFLETPSQFFPFTGLNSLTY |
| | DVSVAQGVTNLHTAIMAQLAAGNEVVVFGTSQSATIATFEMRYLQSLPA |
| | HLRPGLDELSFTLTGNPNRPDGGILTRFGFSIPQLGFTLSGATPADAYPTV |
| | DYAFQYDGVNDFPKYPLNVFATANAIAGILFLHSGLIALPPDLASGVVQP |
| | VSSPDVLTTYILLPSQDLPLLVPLRAIPLLGNPLADLIQPDLRVLVELGYDR |
| | TAHQDVPSPFGLFPDVDWAEVAADLQQGAVQGVNDALSGLGLPPPWQP |
| | ALPRLFSTFSRPGLPVEYLQVPSPSMGRDIKVQFQSGGNNSPAVYLLDGL |
| | RAQDDYNGWDINTPAFEWYYQSGLSIVMPVGGQSSFYSDWYSPACGKA |
| | GCQTYKWETFLTSELPQWLSANRAVKPTGSAAIGLSMAGSSAMILAAYH |
| | PQQFIYAGSLSALLDPSQGMGPSLIGLAMGDAGGYKAADMWGPSSDPA |
| | WERNDPTQQIPKLVANNTRLWVYCGNGTPNELGGANIPAEFLENFVRSS |
| | NLKFQDAYNAAGGHNAVFNFPPNGTHSWEYWGAQLNAMKGDLQSSLG |
| | AG |
| ID125-2 | ID125-2 |
| | MTINYQFGDVDAHGAMIRAQAGSLEAEHQAIISDVLTASDFWGGAGSAA |
| | CQGFITQLGRNFQVIYEQANAHGQKVQAAGNNMAQTDSAVGSSWAGTH |
| | LANGSMSEVMMSEIAGLPIPPIIHYGAIAYAPSGASGKAWHQRTPARAEQ |
| | VALEKCGDKTCKVVSRFTRCGAVAYNGSKYQGGTGLTRRAAEDDAVNR |
| | LEGGRIVNWACNELMTSRFMTDPHAMRDMAGRFEVHAQTVEDEARRM |
| | WASAQNISGAGWSGMAEATSLDTMTQMNQAFRNIVNMLHGVRDGLVR |
| | DANNYEQQEQASQQILSSVDMNFAVLPPEVNSARIFAGAGLGPMLAAAS |
| | AWDGLAEELHAAAGSFASVTTGLAGDAWHGPASLAMTRAASPYVGWL |
| | NTAAGQAAQAAGQARLAASAFEATLAATVSPAMVAANRTRLASLVAAN |
| | LLGQNAPAIAAAEAEYEQIWAQDVAAMFGYHSAASAVATQLAPIQEGLQ |
| | QQLQNVLAQLASGNLGSGNVGVGNIGNDNIGNANIGFGNRGDANIGIGNI |
| | GDRNLGIGNTGNWNIGIGITGNGQIGFGKPANPDVLVVGNGGPGVTALV |
| | MGGTDSLLPLPNIPLLEYAARFITPVHPGYTATFLETPSQFFPFTGLNSLTY |
| | DVSVAQGVTNLHTAIMAQLAAGNEVVVFGTSQSATIATFEMRYLQSLPA |
| | HLRPGLDELSFTLTGNPNRPDGGILTRFGFSIPQLGFTLSGATPADAYPTV |
| | DYAFQYDGVNDFPKYPLNVFATANAIAGILFLHSGLIALPPDLASGVVQP |
| | VSSPDVLTTYILLPSQDLPLLVPLRAIPLLGNPLADLIQPDLRVLVELGYDR |
| | TAHQDVPSPFGLFPDVDWAEVAADLQQGAVQGVNDALSGLGLPPPWQP |
| | ALPRLFSTFSRPGLPVEYLQVPSPSMGRDIKVQFQSGGNNSPAVYLLDGL |
| | RAQDDYNGWDINTPAFEWYYQSGLSIVMPVGGQSSFYSDWYSPACGKA |
| | GCQTYKWETFLTSELPQWLSANRAVKPTGSAAIGLSMAGSSAMILAAYH |
| | PQQFIYAGSLSALLDPSQGMGPSLIGLAMGDAGGYKAADMWGPSSDPA |
| | WERNDPTQQIPKLVANNTRLWVYCGNGTPNELGGANIPAEFLENFVRSS |
| | NLKFQDAYNAAGGHNAVFNFPPNGTHSWEYWGAQLNAMKGDLQSSLG |
| | AG |

Polynucleotide Compositions

The present disclosure, in another aspect, also provides isolated polynucleotides, encoding the fusion polypeptides provided herein.

As used herein, the terms “DNA” and “polynucleotide” and “nucleic acid” refer to a DNA molecule that has been isolated free of total genomic DNA of a particular species. Therefore, a DNA segment encoding a polypeptide refers to a DNA segment that contains one or more coding sequences, yet is substantially isolated away from, or purified free from, total genomic DNA of the species from which the DNA segment is obtained. Included within the terms “DNA segment” and “polynucleotide” are DNA segments and smaller fragments of such segments, and also recombinant vectors, including, for example, plasmids, cosmids, phagemids, phage, viruses, and the like.

As will be understood by those skilled in the art, the polynucleotide sequences of this disclosure can include genomic sequences, extra-genomic and plasmid-encoded sequences and smaller engineered gene segments that express, or may be adapted to express, proteins, polypeptides, peptides, and the like. Such segments may be naturally isolated or modified synthetically by the hand of man.

As will be recognized by the skilled artisan, polynucleotides may be single-stranded (coding or antisense) or double-stranded, and may be DNA (genomic, cDNA or synthetic) or RNA molecules. Additional coding or non-coding sequences may, but need not, be present within a polynucleotide of the present disclosure, and a polynucleotide may, but need not, be linked to other molecules and/or support materials. Polynucleotides may comprise a native sequence (i.e., an endogenous sequence that encodes a Mtb antigen, a NTM antigen, or a portion thereof) or may comprise a variant, or a biological or antigenic functional equivalent of such a sequence. Polynucleotide variants may contain one or more substitutions, additions, deletions and/or insertions, as further described below, such that the immunogenicity of the encoded polypeptide is not diminished, relative to the native protein. The effect on the immunogenicity of the encoded polypeptide may generally be assessed as described herein. The term “variants” also encompasses homologous genes of xenogenic origin.

In additional embodiments, the present disclosure provides isolated polynucleotides comprising various lengths of contiguous stretches of sequence identical to or complementary to one or more of the sequences disclosed herein. For example, polynucleotides are provided by this disclosure that comprise at least about 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200 500; 500 1,000, and the like.

The polynucleotides of the present disclosure, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a polynucleotide fragment of almost any length may be employed, where the total length may be limited by the ease of preparation and use in the intended recombinant DNA protocol.

Moreover, it will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present disclosure, for example polynucleotides that are optimized for human and/or primate codon selection. Further, alleles of the genes comprising the polynucleotide sequences provided herein are within the scope of the present disclosure. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Polynucleotides encoding Mtb antigens and NTM antigens; and polynucleotides encoding the fusion polypeptides provided herein may be prepared, manipulated and/or expressed using any of a variety of well-established techniques known and available in the art.

For example, polynucleotide sequences or fragments thereof which encode the fusion polypeptides provided herein, or functional equivalents thereof, may be used in recombinant DNA molecules to direct expression of a polypeptide in appropriate host cells. Due to the inherent degeneracy of the genetic code, other DNA sequences that encode substantially the same or a functionally equivalent amino acid sequence may be produced, and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

Moreover, the polynucleotide sequences of the present disclosure can be engineered using methods generally known in the art in order to alter polypeptide encoding sequences for a variety of reasons, including but not limited to, alterations which modify the cloning, processing, expression and/or immunogenicity of the gene product.

In order to express a desired polypeptide, a nucleotide sequence encoding the polypeptide, or a functional equivalent, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989). A variety of expression vector/host systems are known and may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses can be used. If it is necessary to generate a cell line that contains multiple copies of the sequence encoding a polypeptide, vectors based on SV40 or EBV may be advantageously used with an appropriate selectable marker.

In bacterial systems, a number of expression vectors may be selected depending upon the use intended for the expressed polypeptide. For example, when large quantities are needed, vectors which direct high-level expression of fusion proteins that are readily purified may be used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of (3-galactosidase so that a hybrid protein is produced; pIN vectors (Van Heeke & Schuster, J. Biol. Chem. 264:5503 5509 (1989)); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al., Methods Enzymol. 153:516-544 (1987).

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, EMBO J. 6:307-311 (1987)). Alternatively, plant promoters such as the small subunit of RUBISCO or heat shock promoters may be used (Coruzzi et al., EMBO J. 3:1671-1680 (1984); Broglie et al., Science 224:838-843 (1984); and Winter et al., Results Probl. Cell Differ. 17:85-105 (1991)). These constructs can be introduced into plant cells by direct DNA transformation or pathogen-mediated transfection. Such techniques are described in a number of generally available reviews (see, e.g., Hobbs in McGraw Hill, Yearbook of Science and Technology, pp. 191-196 (1992)).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard et al., Proc. Natl. Acad. Sci. U.S.A. 91:3224-3227 (1994)).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a nonessential E1 or E3 region of the viral genome may be used to obtain a viable virus which is capable of expressing the polypeptide in infected host cells (Logan & Shenk, Proc. Natl. Acad. Sci. U.S.A. 81:3655-3659 (1984)). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf. et al., Results Probl. Cell Differ. 20:125-162 (1994)).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, HeLa, MDCK, HEK293, and W138, which have specific cellular machinery and characteristic mechanisms for such post-translational activities, may be chosen to ensure the correct modification and processing of the foreign protein.

For long-term, high-yield production of recombinant proteins, stable expression is often desired. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler et al., Cell 11:223-232 (1977)) and adenine phosphoribosyltransferase (Lowy et al., Cell 22:817-823 (1990)) genes which can be employed in tk- or aprt-cells, respectively. Also, antimetabolite, antibiotic or herbicide resistance can be used as the basis for selection; for example, dhfr which confers resistance to methotrexate (Wigler et al., Proc. Natl. Acad. Sci. U.S.A. 77:3567-70 (1980)); npt, which confers resistance to the aminoglycosides, neomycin and G-418 (ColbereGarapin et al., J. Mol. Biol. 150:1-14 (1981)); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. Sci. U.S.A. 85:8047-51 (1988)). The use of visible markers has gained popularity with such markers as anthocyanins, (3-glucuronidase and its substrate GUS, and luciferase and its substrate luciferin, being widely used not only to identify transformants, but also to quantify the amount of transient or stable protein expression attributable to a specific vector system (Rhodes et al., Methods Mol. Biol. 55:121-131 (1995)).

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (MA), and fluorescence activated cell sorting (FACS). These and other assays are described, among other places, in Hampton et al., Serological Methods, a Laboratory Manual (1990) and Maddox et al., J. Exp. Med. 158:1211-1216 (1983).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the disclosure may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification of soluble proteins.

In addition to recombinant production methods, polypeptides of the disclosure, and fragments thereof, may be produced by direct peptide synthesis using solid-phase techniques (Merrifield, J. Am. Chem. Soc. 85:2149-2154 (1963)). Protein synthesis may be performed using manual techniques or by automation. Automated synthesis may be achieved, for example, using Applied Biosystems 43 1 A Peptide Synthesizer (Perkin Elmer). Alternatively, various fragments may be chemically synthesized separately and combined using chemical methods to produce the full-length molecule.

Table 3 provides exemplary nucleotide sequences encoding for exemplary Mtb antigens used to construct the fusion polypeptides provided herein. Likewise, Table 4 provides exemplary nucleotide sequences encoding for exemplary fusion polypeptides of the present invention.

TABLE 3
Exemplary Nucleotide Sequences Encoding Antigens

| | |
|---|---|
| Rv0496-b | Rv0496-b |
| | GTCG ATGCCCACCG CGGCGGCCAC CCGACCCCGA TGAGCTCGAC |
| | GAAGGCCACGCTGCGGCTGG CCGAGGCCAC CGACAGCTCG GGCAAGATCA CCAAGCGCGG |
| | AGCCGACAAGCTGATTTCCA CCATCGACGA ATTCGCCAAG ATTGCCATCA GCTCGGGCTG |
| | TGCCGAGCTGATGGCCTTCG CCACGTCGGC GGTCCGCGAC GCCGAGAATT CCGAGGACGT |
| | CCTGTCCCGGGTGCGCAAAG AGACCGGTGT CGAGTTGCAG GCGCTGCGTG GGGAGGACGA |
| | GTCACGGCTGACCTTCCTGG CCGTGCGACG ATGGTACGGG TGGAGCGCTG GGCGCATCCT |
| | CAACCTCGACATCGGCGGCG GCTCGCTGGA AGTGTCCAGT GGCGTGGACG AGGAGCCCGA |
| | GATTGCGTTATCGCTGCCCC TGGGCGCCGG ACGGTTGACC CGAGAGTGGC TGCCCGACGA |
| | TCCGCCGGGCCGGCGCCGGG TGGCGATGCT GCGAGACTGG CTGGATGCCG AGCTGGCCGA |
| | GCCCAGTGTGACCGTCCTGG AAGCCGGCAG CCCCGACCTG GCGGTCGCAA CGTCGAAGAC |
| | GTTTCGCTCGTTGGCGCGAC TAACCGGTGC GGCCCCATCC ATGGCCGGGC CGCGGGTGAA |
| | GAGGACCCTAACGGCAAATG GTCTGCGGCA ACTCATCGCG TTTATCTCTA GGATGACGGC |
| | GGTTGACCGTGCAGAACTGG AAGGGGTAAG CGCCGACCGA GCGCCGCAGA TTGTGGCCGG |
| | CGCCCTGGTGGCAGAGGCGA GCATGCGAGC ACTGTCGATA GAAGCGGTGG AAATCTGCCC |
| | GTGGGCGCTGCGGGAAGGTC TCATCTTGCG CAAACTCGAC AGCGAAGCCG ACGGAACCGC |
| | CCTCATCGAGTCTTCGTCTG TGCACACTTC GGTGCGTGCC GTCGGAGGTC AGCCAGCTGA |
| | TCGGAACGCGGCCAACCGAT CGAGAGGCAG CAAACCA |
| Rv1813-b | Rv1813-b |
| | CATCTCGCCA ACGGTTCGAT GTCGGAAGTC ATGATGTCGG AAATTGCCGG GTTGCCTATC |
| | CCTCCGATTA TCCATTACGG GGCGATTGCC TATGCCCCCA GCGGCGCGTC GGGCAAAGCG |
| | TGGCACCAGC GCACACCGGC GCGAGCAGAG CAAGTCGCAC TAGAAAAGTG |
| | CGGTGACAAG ACTTGCAAAG TGGTTAGTCG CTTCACCAGG TGCGGCGCGG TCGCCTACAA |
| | CGGCTCGAAA TACCAAGGCG GAACCGGACT CACGCGCCGC GCGGCAGAAG |
| | ACGACGCCGT GAACCGACTC GAAGGCGGGC GGATCGTCAA CTGGGCGTGC AA |
| Rv1886-b | Rv1886-b |
| | TTCTCCCGGCCGGGGCTGCCGGTCGAGTACCTGCAGGTGCCGTCGCCGTCGATGGGCCGCGA |
| | CATCAAGGTTCAGTTCCAGAGCGGTGGGAACAACTCACCTGCGGTTTATCTGCTCGACGGCC |
| | TGCGCGCCCAAGACGACTACAACGGCTGGGATATCAACACCCCGGCGTTCGAGTGGTACTAC |
| | CAGTCGGGACTGTCGATAGTCATGCCGGTCGGCGGGCAGTCCAGCTTCTACAGCGACTGGTA |
| | CAGCCCGGCCTGCGGTAAGGCTGGCTGCCAGACTTACAAGTGGGAAACCTTCCTGACCAGCG |
| | AGCTGCCGCAATGGTTGTCCGCCAACAGGGCCGTGAAGCCCACCGGCAGCGCTGCAATCGGC |
| | TTGTCGATGGCCGGCTCGTCGGCAATGATCTTGGCCGCCTACCACCCCCAGCAGTTCATCTA |
| | CGCCGGCTCGCTGTCGGCCCTGCTGGACCCCTCTCAGGGGATGGGGCCTAGCCTGATCGGCC |
| | TCGCGATGGGTGACGCCGGCGGTTACAAGGCCGCAGACATGTGGGGTCCCTCGAGTGACCC |
| | GGCATGGGAGCGCAACGACCCTACGCAGCAGATCCCCAAGCTGGTCGCAAACAACACCCGG |
| | CTATGGGTTTATTGCGGGAACGGCACCCCGAACGAGTTGGGCGGTGCCAACATACCCGCCG |
| | AGTTCTTGGAGAACTTCGTTCGTAGCAGCAACCTGAAGTTCCAGGATGCGTACAACGCCGC |
| | GGGCGGGCACAACGCCGTGTTCAACTTCCCGCCCAACGGCACGCAC AGCTGGGAGT |
| | ACTGGGGCGC TCAGCTCAACGCCATGAAGG GTGACCTGCA GAGTTCGTTA GGCGCCGGC |
| Rv2608-a | Rv2608-a |
| | atgaatt tcgccgtttt gccgccggag gtgaattcgg cgcgcatatt cgccggtgcg |
| | ggcctgggcc caatgctggc ggcggcgtcg gcctgggacg ggttggccga ggagttgcat |
| | gccgcggcgg gctcgttcgc gtcggtgacc accgggttgg cgggcgacgc gtggcatggt |
| | ccggcgtcgc tggcgatgac ccgcgcggcc agcccgtatg tggggtggtt gaacacggcg |
| | gcgggtcagg ccgcgcaggc ggccggccag gcgcggctag cggcgagcgc gttcgaggcg |
| | acgctggcgg ccaccgtgtc tccagcgatg gtcgcggcca accggacacg gctggcgtcg |
| | ctggtggcag ccaacttgct gggccagaac gccccggcga tcgcggccgc ggaggctgaa |
| | tacgagcaga tatgggccca ggacgtggcc gcgatgttcg gctatcactc cgccgcgtcg |
| | gcggtggcca cgcagctggc gcctattcaa gagggtttgc agcagcagct gcaaaacgtg |
| | ctggcccagt tggctagcgg gaacctgggc agcggaaatg tgggcgtcgg caacatcggc |
| | aacgacaaca ttggcaacgc aaacatcggc ttcggaaatc gaggcgacgc caacatcggc |
| | atcgggaata tcggcgacag aaacctcggc attgggaaca ccggcaattg gaatatcggc |
| | atcggcatca ccggcaacgg acaaatcggc ttcggcaagc ctgccaaccc cgacgtcttg |
| | gtggtgggca acggcggccc gggagtaacc gcgttggtca tgggcggcac cgacagccta |
| | ctgccgctgc ccaacatccc cttactcgag tacgctgcgc ggttcatcac ccccgtgcat |
| | cccggataca ccgctacgtt cctggaaacg ccatcgcagt ttttcccatt caccgggctg |
| | aatagcctga cctatgacgt ctccgtggcc cagggcgtaa cgaatctgca caccgcgatc |
| | atggcgcaac tcgcggcggg aaacgaagtc gtcgtcttcg gcacctccca aagcgccacg |
| | atagccacct tcgaaatgcg ctatctgcaa tccctgccag cacacctgcg tccgggtctc |
| | gacgaattgt cctttacgtt gaccggcaat cccaaccggc ccgacggtgg cattcttacg |
| | cgttttggct tctccatacc gcagttgggt ttcacattgt ccggcgcgac gcccgccgac |
| | gcctacccca ccgtcgatta cgcgttccag tacgacggcg tcaacgactt ccccaaatac |
| | ccgctgaatg tcttcgcgac cgccaacgcg atcgcgggca tccttttcct gcactccggg |
| | ttgattgcgt tgccgcccga tcttgcctcg ggcgtggttc aaccggtgtc ctcaccggac |
| | gtcctgacca cctacatcct gctgcccagc caagatctgc cgctgctggt cccgctgcgt |
| | gctatccccc tgctgggaaa cccgcttgcc gacctcatcc agccggactt gcgggtgctc |
| | gtcgagttgg gttatgaccg caccgcccac caggacgtgc ccagcccgtt cggactgttt |
| | ccggacgtcg attgggccga ggtggccgcg gacctgcagc aaggcgccgt gcaaggcgtc |
| | aacgacgccc tgtccggact ggggctgccg ccgccgtggc agccggcgct accccgactt |
| | ttc |
| Rv2608-b | Rv2608-b |
| | aatt tcgccgtttt gccgccggag gtgaattcgg cgcgcatatt cgccggtgcg |
| | ggcctgggcc caatgctggc ggcggcgtcg gcctgggacg ggttggccga ggagttgcat |
| | gccgcggcgg gctcgttcgc gtcggtgacc accgggttgg cgggcgacgc gtggcatggt |
| | ccggcgtcgc tggcgatgac ccgcgcggcc agcccgtatg tggggtggtt gaacacggcg |
| | gcgggtcagg ccgcgcaggc ggccggccag gcgcggctag cggcgagcgc gttcgaggcg |
| | acgctggcgg ccaccgtgtc tccagcgatg gtcgcggcca accggacacg gctggcgtcg |
| | ctggtggcag ccaacttgct gggccagaac gccccggcga tcgcggccgc ggaggctgaa |
| | tacgagcaga tatgggccca ggacgtggcc gcgatgttcg gctatcactc cgccgcgtcg |
| | gcggtggcca cgcagctggc gcctattcaa gagggtttgc agcagcagct gcaaaacgtg |
| | ctggcccagt tggctagcgg gaacctgggc agcggaaatg tgggcgtcgg caacatcggc |
| | aacgacaaca ttggcaacgc aaacatcggc ttcggaaatc gaggcgacgc caacatcggc |
| | atcgggaata tcggcgacag aaacctcggc attgggaaca ccggcaattg gaatatcggc |
| | atcggcatca ccggcaacgg acaaatcggc ttcggcaagc ctgccaaccc cgacgtcttg |
| | gtggtgggca acggcggccc gggagtaacc gcgttggtca tgggcggcac cgacagccta |
| | ctgccgctgc ccaacatccc cttactcgag tacgctgcgc ggttcatcac ccccgtgcat |
| | cccggataca ccgctacgtt cctggaaacg ccatcgcagt ttttcccatt caccgggctg |
| | aatagcctga cctatgacgt ctccgtggcc cagggcgtaa cgaatctgca caccgcgatc |
| | atggcgcaac tcgcggcggg aaacgaagtc gtcgtcttcg gcacctccca aagcgccacg |
| | atagccacct tcgaaatgcg ctatctgcaa tccctgccag cacacctgcg tccgggtctc |
| | gacgaattgt cctttacgtt gaccggcaat cccaaccggc ccgacggtgg cattcttacg |
| | cgttttggct tctccatacc gcagttgggt ttcacattgt ccggcgcgac gcccgccgac |
| | gcctacccca ccgtcgatta cgcgttccag tacgacggcg tcaacgactt ccccaaatac |
| | ccgctgaatg tcttcgcgac cgccaacgcg atcgcgggca tccttttcct gcactccggg |
| | ttgattgcgt tgccgcccga tcttgcctcg ggcgtggttc aaccggtgtc ctcaccggac |
| | gtcctgacca cctacatcct gctgcccagc caagatctgc cgctgctggt cccgctgcgt |
| | gctatccccc tgctgggaaa cccgcttgcc gacctcatcc agccggactt gcgggtgctc |
| | gtcgagttgg gttatgaccg caccgcccac caggacgtgc ccagcccgtt cggactgttt |
| | ccggacgtcg attgggccga ggtggccgcg gacctgcagc aaggcgccgt gcaaggcgtc |
| | aacgacgccc tgtccggact ggggctgccg ccgccgtggc agccggcgct accccgactt |
| | TTC |
| Rv2875-d | Rv2875-d |
| | ggcg atctggtgag cccgggctgc gcggaatacg cggcagccaa tcccactggg |
| | ccggcctcgg tgcagggaat gtcgcaggac ccggtcgcgg tggcggcctc gaacaatccg |
| | gagttgacaa cgctgacggc tgcactgtcg ggccagctca atccgcaagt aaacctggtg |
| | gacaccctca acagcggtca gtacacggtg ttcgcaccga ccaacgcggc atttagcaag |
| | ctgccggcat ccacgatcga cgagctcaag accaattcgt cactgctgac cagcatcctg |
| | acctaccacg tagtggccgg ccaaaccagc ccggccaacg tcgtcggcac ccgtcagacc |
| | ctccagggcg ccagcgtgac ggtgaccggt cagggtaaca gcctcaaggt cggtaacgcc |
| | gacgtcgtct gtggtggggt gtctaccgcc aacgcgacgg tgtacatgat tgacagcgtg |
| | ctaatgcctc cggcg |
| Rv3619 | Rv3619 |
| | atgacca tcaactatca attcggggac gtcgacgctc acggcgccat gatccgcgct |
| | caggccgggt cgctggaggc cgagcatcag gccatcattt ctgatgtgtt gaccgcgagt |
| | gacttttggg gcggcgccgg ttcggcggcc tgccaggggt tcattaccca gctgggccgt |
| | aacttccagg tgatctacga gcaggccaac gcccacgggc agaaggtgca ggctgccggc |
| | aacaacatgg cacaaaccga cagcgccgtc ggctccagct gggcc |
| Rv3620 | Rv3620 |
| | atgacct cgcgttttat gacggatccg cacgcgatgc gggacatggc gggccgtttt |
| | gaggtgcacg cccagacggt ggaggacgag gctcgccgga tgtgggcgtc cgcgcaaaac |
| | atttccggcg cgggctggag tggcatggcc gaggcgacct cgctagacac catgacccag |
| | atgaatcagg cgtttcgcaa catcgtgaac atgctgcacg gggtgcgtga cgggctggtt |
| | cgcgacgcca acaactacga acagcaagag caggcctccc agcagatcct cagcagc |
| Rv3810-b | Rv3810-b |
| | agtcct tgtgcatatt ttcttgtcta cgaatcaacc |
| | gaaacgaccg agcggcccga gcaccatgaa ttcaagcagg cggcggtgtt gaccgacctg |
| | cccggcgagc tgatgtccgc gctatcgcag gggttgtccc agttcgggat caacataccg |
| | ccggtgccca gcctgaccgg gagcggcgat gccagcacgg gtctaaccgg tcctggcctg |
| | actagtccgg gattgaccag cccgggattg accagcccgg gcctcaccga ccctgccctt |
| | accagtccgg gcctgacgcc aaccctgccc ggatcactcg ccgcgcccgg caccaccctg |
| | gcgccaacgc ccggcgtggg ggccaatccg gcgctcacca accccgcgct gaccagcccg |
| | accggggcga cgccgggatt gaccagcccg acgggtttgg atcccgcgct gggcggcgcc |
| | aacgaaatcc cgattacgac gccggtcgga ttggatcccg gggctgacgg cacctatccg |
| | atcctcggtg atccaacact ggggaccata ccgagcagcc ccgccaccac ctccaccggc |
| | ggcggcggtc tcgtcaacga cgtgatgcag gtggccaacg agttgggcgc cagtcaggct |
| | atcgacctgc taaaaggtgt gctaatgccg tcgatcatgc aggccgtcca gaatggcggc |
| | gcggccgcgc cggcagccag cccgccggtc ccgcccatcc ccgcggccgc ggcggtgcca |
| | ccgacggacc caatcaccgt gccggtcgcc |

TABLE 4
Exemplary Nucleotide Sequences Encoding Fusion Polypeptides

| | |
|---|---|
| ID58 | ID58 |
| | ggtaccc atctcgccaa cggttcgatg tcggaagtca tgatgtcgga aattgccggg |
| | ttgcctatcc ctccgattat ccattacggg gcgattgcct atgcccccag cggcgcgtcg |
| | ggcaaagcgt ggcaccagcg cacaccggcg cgagcagagc aagtcgcact agaaaagtgc |
| | ggtgacaaga cttgcaaagt ggttagtcgc ttcaccaggt gcggcgcggt cgcctacaac |
| | ggctcgaaat accaaggcgg aaccggactc acgcgccgcg cggcagaaga cgacgccgtg |
| | aaccgactcg aaggcgggcg gatcgtcaac tgggcgtgca acgagctcat gacctcgcgt |
| | tttatgacgg atccgcacgc gatgcgggac atggcgggcc gttttgaggt gcacgcccag |
| | acggtggagg acgaggctcg ccggatgtgg gcgtccgcgc aaaacatctc gggcgcgggc |
| | tggagtggca tggccgaggc gacctcgcta gacaccatga cccagatgaa tcaggcgttt |
| | cgcaacatcg tgaacatgct gcacggggtg cgtgacgggc tggttcgcga cgccaacaac |
| | tacgaacagc aagagcaggc ctcccagcag atcctcagca gcgtcgacgt ggtcgatgcc |
| | caccgcggcg gccacccgac cccgatgagc tcgacgaagg ccacgctgcg gctggccgag |
| | gccaccgaca gctcgggcaa gatcaccaag cgcggagccg acaagctgat ttccaccatc |
| | gacgaattcg ccaagattgc catcagctcg ggctgtgccg agctgatggc cttcgccacg |
| | tcggcggtcc gcgacgccga gaattccgag gacgtcctgt cccgggtgcg caaagagacc |
| | ggtgtcgagt tgcaggcgct gcgtggggag gacgagtcac ggctgacctt cctggccgtg |
| | cgacgatggt acgggtggag cgctgggcgc atcctcaacc tcgacatcgg cggcggctcg |
| | ctggaagtgt ccagtggcgt ggacgaggag cccgagattg cgttatcgct gcccctgggc |
| | gccggacggt tgacccgaga gtggctgccc gacgatccgc cgggccggcg ccgggtggcg |
| | atgctgcgag actggctgga tgccgagctg gccgagccca gtgtgaccgt cctggaagcc |
| | ggcagccccg acctggcggt cgcaacgtcg aagacgtttc gctcgttggc gcgactaacc |
| | ggtgcggccc catccatggc cgggccgcgg gtgaagagga ccctaacggc aaatggtctg |
| | cggcaactca tcgcgtttat ctctaggatg acggcggttg accgtgcaga actggaaggg |
| | gtaagcgccg accgagcgcc gcagattgtg gccggcgccc tggtggcaga ggcgagcatg |
| | cgagcactgt cgatagaagc ggtggaaatc tgcccgtggg cgctgcggga aggtctcatc |
| | ttgcgcaaac tcgacagcga agccgacgga accgccctca tcgagtcttc gtctgtgcac |
| | acttcggtgc gtgccgtcgg aggtcagcca gctgatcgga acgcggccaa ccgatcgaga |
| | ggcagcaaac caagtact |
| ID69 | ID69 |
| | gacgaca tcgattggga cgccatcgcg caatgcgaat ccggcggcaa ttgggcggcc |
| | aacaccggta acgggttata cggtggtctg cagatcagcc aggcgacgtg ggattccaac |
| | ggtggtgtcg ggtcgccggc ggccgcgagt ccccagcaac agatcgaggt cgcagacaac |
| | attatgaaaa cccaaggccc gggtgcgtgg ccgaaatgta gttcttgtag tcagggagac |
| | gcaccgctgg gctcgctcac ccacatcctg acgttcctcg cggccgagac tggaggttgt |
| | tcggggagca gggacgatgg tacccatctc gccaacggtt cgatgtcgga agtcatgatg |
| | tcggaaattg ccgggttgcc tatccctccg attatccatt acggggcgat tgcctatgcc |
| | cccagcggcg cgtcgggcaa agcgtggcac cagcgcacac cggcgcgagc agagcaagtc |
| | gcactagaaa agtgcggtga caagacttgc aaagtggtta gtcgcttcac caggtgcggc |
| | gcggtcgcct acaacggctc gaaataccaa ggcggaaccg gactcacgcg ccgcgcggca |
| | gaagacgacg ccgtgaaccg actcgaaggc gggcggatcg tcaactgggc gtgcaacgag |
| | ctcatgacct cgcgttttat gacggatccg cacgcgatgc gggacatggc gggccgtttt |
| | gaggtgcacg cccagacggt ggaggacgag gctcgccgga tgtgggcgtc cgcgcaaaac |
| | atctcgggcg cgggctggag tggcatggcc gaggcgacct cgctagacac catgacccag |
| | atgaatcagg cgtttcgcaa catcgtgaac atgctgcacg gggtgcgtga cgggctggtt |
| | cgcgacgcca acaactacga acagcaagag caggcctccc agcagatcct cagcagcgtc |
| | gacatggtcg atgcccaccg cggcggccac ccgaccccga tgagctcgac gaaggccacg |
| | ctgcggctgg ccgaggccac cgacagctcg ggcaagatca ccaagcgcgg agccgacaag |
| | ctgatttcca ccatcgacga attcgccaag attgccatca gctcgggctg tgccgagctg |
| | atggccttcg ccacgtcggc ggtccgcgac gccgagaatt ccgaggacgt cctgtcccgg |
| | gtgcgcaaag agaccggtgt cgagttgcag gcgctgcgtg gggaggacga gtcacggctg |
| | accttcctgg ccgtgcgacg atggtacggg tggagcgctg ggcgcatcct caacctcgac |
| | atcggcggcg gctcgctgga agtgtccagt ggcgtggacg aggagcccga gattgcgtta |
| | tcgctgcccc tgggcgccgg acggttgacc cgagagtggc tgcccgacga tccgccgggc |
| | cggcgccggg tggcgatgct gcgagactgg ctggatgccg agctggccga gcccagtgtg |
| | accgtcctgg aagccggcag ccccgacctg gcggtcgcaa cgtcgaagac gtttcgctcg |
| | ttggcgcgac taaccggtgc ggccccatcc atggccgggc cgcgggtgaa gaggacccta |
| | acggcaaatg gtctgcggca actcatcgcg tttatctcta ggatgacggc ggttgaccgt |
| | gcagaactgg aaggggtaag cgccgaccga gcgccgcaga ttgtggccgg cgccctggtg |
| | gcagaggcga gcatgcgagc actgtcgata gaagcggtgg aaatctgccc gtgggcgctg |
| | cgggaaggtc tcatcttgcg caaactcgac agcgaagccg acggaaccgc cctcatcgag |
| | tcttcgtctg tgcacacttc ggtgcgtgcc gtcggaggtc agccagctga tcggaacgcg |
| | gccaaccgat cgagaggcag caaaccaagt act |
| ID71 | ID71 |
| | catatgatga ccatcaacta tcaattcggg gacgtcgacg ctcacggcgc catgatccgc |
| | gctcaggccg ggtcgctgga ggccgagcat caggccatca tttctgatgt gttgaccgcg |
| | agtgactttt ggggcggcgc cggttcggcg gcctgccagg ggttcattac ccagctgggc |
| | cgtaacttcc aggtgatcta cgagcaggcc aacgcccacg ggcagaaggt gcaggctgcc |
| | ggcaacaaca tggcacaaac cgacagcgcc gtcggctcca gctgggccgg taccgacgac |
| | atcgattggg acgccatcgc gcaatgcgaa tccggcggca attgggcggc caacaccggt |
| | aacgggttat acggtggtct gcagatcagc caggcgacgt gggattccaa cggtggtgtc |
| | gggtcgccgg cggccgcgag tccccagcaa cagatcgagg tcgcagacaa cattatgaaa |
| | acccaaggcc cgggtgcgtg gccgaaatgt agttcttgta gtcagggaga cgcaccgctg |
| | ggctcgctca cccacatcct gacgttcctc gcggccgaga ctggaggttg ttcggggagc |
| | agggacgatg gatccgtggt ggatttcggg gcgttaccac cggagatcaa ctccgcgagg |
| | atgtacgccg gcccgggttc ggcctcgctg gtggccgccg cgaagatgtg ggacagcgtg |
| | gcgagtgacc tgttttcggc cgcgtcggcg tttcagtcgg tggtctgggg tctgacggtg |
| | gggtcgtgga taggttcgtc ggcgggtctg atggcggcgg cggcctcgcc gtatgtggcg |
| | tggatgagcg tcaccgcggg gcaggcccag ctgaccgccg cccaggtccg ggttgctgcg |
| | gcggcctacg agacagcgta taggctgacg gtgcccccgc cggtgatcgc cgagaaccgt |
| | accgaactga tgacgctgac cgcgaccaac ctcttggggc aaaacacgcc ggcgatcgag |
| | gccaatcagg ccgcatacag ccagatgtgg ggccaagacg cggaggcgat gtatggctac |
| | gccgccacgg cggcgacggc gaccgaggcg ttgctgccgt tcgaggacgc cccactgatc |
| | accaaccccg gcggggaatt cttctcccgg ccggggctgc cggtcgagta cctgcaggtg |
| | ccgtcgccgt cgatgggccg cgacatcaag gttcagttcc agagcggtgg gaacaactca |
| | cctgcggttt atctgctcga cggcctgcgc gcccaagacg actacaacgg ctgggatatc |
| | aacaccccgg cgttcgagtg gtactaccag tcgggactgt cgatagtcat gccggtcggc |
| | gggcagtcca gcttctacag cgactggtac agcccggcct gcggtaaggc tggctgccag |
| | acttacaagt gggaaacctt cctgaccagc gagctgccgc aatggttgtc cgccaacagg |
| | gccgtgaagc ccaccggcag cgctgcaatc ggcttgtcga tggccggctc gtcggcaatg |
| | atcttggccg cctaccaccc ccagcagttc atctacgccg gctcgctgtc ggccctgctg |
| | gacccctctc aggggatggg gcctagcctg atcggcctcg cgatgggtga cgccggcggt |
| | tacaaggccg cagacatgtg gggtccctcg agtgacccgg catgggagcg caacgaccct |
| | acgcagcaga tccccaagct ggtcgcaaac aacacccggc tatgggttta ttgcgggaac |
| | ggcaccccga acgagttggg cggtgccaac atacccgccg agttcttgga gaacttcgtt |
| | cgtagcagca acctgaagtt ccaggatgcg tacaacgccg cgggcgggca caacgccgtg |
| | ttcaacttcc cgcccaacgg cacgcacagc tgggagtact ggggcgctca gctcaacgcc |
| | atgaagggtg acctgcagag ttcgttaggc gccggc |
| ID83-1 | ID83-1 and ID83-2 |
| and | ggtaccc atctcgccaa cggttcgatg tcggaagtca tgatgtcgga aattgccggg |
| ID83-2 | ttgcctatcc ctccgattat ccattacggg gcgattgcct atgcccccag cggcgcgtcg |
| | ggcaaagcgt ggcaccagcg cacaccggcg cgagcagagc aagtcgcact agaaaagtgc |
| | ggtgacaaga cttgcaaagt ggttagtcgc ttcaccaggt gcggcgcggt cgcctacaac |
| | ggctcgaaat accaaggcgg aaccggactc acgcgccgcg cggcagaaga cgacgccgtg |
| | aaccgactcg aaggcgggcg gatcgtcaac tgggcgtgca acgagctcat gacctcgcgt |
| | tttatgacgg atccgcacgc gatgcgggac atggcgggcc gttttgaggt gcacgcccag |
| | acggtggagg acgaggctcg ccggatgtgg gcgtccgcgc aaaacatctc gggcgcgggc |
| | tggagtggca tggccgaggc gacctcgcta gacaccatga cccagatgaa tcaggcgttt |
| | cgcaacatcg tgaacatgct gcacggggtg cgtgacgggc tggttcgcga cgccaacaac |
| | tacgaacagc aagagcaggc ctcccagcag atcctcagca gcgtcgac [symbol] caatttcgcc |
| | gttttgccgc cggaggtgaa ttcggcgcgc atattcgccg gtgcgggcct gggcccaatg |
| | ctggcggcgg cgtcggcctg ggacgggttg gccgaggagt tgcatgccgc ggcgggctcg |
| | ttcgcgtcgg tgaccaccgg gttggcgggc gacgcgtggc atggtccggc gtcgctggcg |
| | atgacccgcg cggccagccc gtatgtgggg tggttgaaca cggcggcggg tcaggccgcg |
| | caggcggccg gccaggcgcg gctagcggcg agcgcgttcg aggcgacgct ggcggccacc |
| | gtgtctccag cgatggtcgc ggccaaccgg acacggctgg cgtcgctggt ggcagccaac |
| | ttgctgggcc agaacgcccc ggcgatcgcg gccgcggagg ctgaatacga gcagatatgg |
| | gcccaggacg tggccgcgat gttcggctat cactccgccg cgtcggcggt ggccacgcag |
| | ctggcgccta ttcaagaggg tttgcagcag cagctgcaaa acgtgctggc ccagttggct |
| | agcgggaacc tgggcagcgg aaatgtgggc gtcggcaaca tcggcaacga caacattggc |
| | aacgcaaaca tcggcttcgg aaatcgaggc gacgccaaca tcggcatcgg gaatatcggc |
| | gacagaaacc tcggcattgg gaacaccggc aattggaata tcggcatcgg catcaccggc |
| | aacggacaaa tcggcttcgg caagcctgcc aaccccgacg tcttggtggt gggcaacggc |
| | ggcccgggag taaccgcgtt ggtcatgggc ggcaccgaca gcctactgcc gctgcccaac |
| | atccccttac tcgagtacgc tgcgcggttc atcacccccg tgcatcccgg atacaccgct |
| | acgttcctgg aaacgccatc gcagtttttc ccattcaccg ggctgaatag cctgacctat |
| | gacgtctccg tggcccaggg cgtaacgaat ctgcacaccg cgatcatggc gcaactcgcg |
| | gcgggaaacg aagtcgtcgt cttcggcacc tcccaaagcg ccacgatagc caccttcgaa |
| | atgcgctatc tgcaatccct gccagcacac ctgcgtccgg gtctcgacga attgtccttt |
| | acgttgaccg gcaatcccaa ccggcccgac ggtggcattc ttacgcgttt tggcttctcc |
| | ataccgcagt tgggtttcac attgtccggc gcgacgcccg ccgacgccta ccccaccgtc |
| | gattacgcgt tccagtacga cggcgtcaac gacttcccca aatacccgct gaatgtcttc |
| | gcgaccgcca acgcgatcgc gggcatcctt ttcctgcact ccgggttgat tgcgttgccg |
| | cccgatcttg cctcgggcgt ggttcaaccg gtgtcctcac cggacgtcct gaccacctac |
| | atcctgctgc ccagccaaga tctgccgctg ctggtcccgc tgcgtgctat ccccctgctg |
| | ggaaacccgc ttgccgacct catccagccg gacttgcggg tgctcgtcga gttgggttat |
| | gaccgcaccg cccaccagga cgtgcccagc ccgttcggac tgtttccgga cgtcgattgg |
| | gccgaggtgg ccgcggacct gcagcaaggc gccgtgcaag gcgtcaacga cgccctgtcc |
| | ggactggggc tgccgccgcc gtggcagccg gcgctacccc gacttttcag tact |
| | [symbol] can encode I or M |
| ID87 | ID87 |
| | atgggcgatctggtgagcccgggctgcgcggaatacg |
| | cggcagccaatcccactgggccggcctcggtgcaggg |
| | aatgtcgcaggacccggtcgcggtggcggcctcgaac |
| | aatccggagttgacaacgctgacggctgcactgtcgg |
| | gccagctcaatccgcaagtaaacctggtggacaccct |
| | caacagcggtcagtacacggtgttcgcaccgaccaac |
| | gcggcatttagcaagctgccggcatccacgatcgacg |
| | agctcaagaccaattcgtcactgctgaccagcatcct |
| | gacctaccacgtagtggccggccaaaccagcccggcc |
| | aacgtcgtcggcacccgtcagaccctccagggcgcca |
| | gcgtgacggtgaccggtcagggtaacagcctcaaggt |
| | cggtaacgccgacgtcgtctgtggtggggtgtctacc |
| | gccaacgcgacggtgtacatgattgacagcgtgctaa |
| | tgcctccggcgggatccgtggtggatttcggggcgtt |
| | accaccggagatcaactccgcgaggatgtacgccggc |
| | ccgggttcggcctcgctggtggccgccgcgaagatgt |
| | gggacagcgtggcgagtgacctgttttcggccgcgtc |
| | ggcgtttcagtcggtggtctggggtctgacggtgggg |
| | tcgtggataggttcgtcggcgggtctgatggcggcgg |
| | cggcctcgccgtatgtggcgtggatgagcgtcaccgc |
| | ggggcaggcccagctgaccgccgcccaggtccgggtt |
| | gctgcggcggcctacgagacagcgtataggctgacgg |
| | tgcccccgccggtgatcgccgagaaccgtaccgaact |
| | gatgacgctgaccgcgaccaacctcttggggcaaaac |
| | acgccggcgatcgaggccaatcaggccgcatacagcc |
| | agatgtggggccaagacgcggaggcgatgtatggcta |
| | cgccgccacggcggcgacggcgaccgaggcgttgctg |
| | ccgttcgaggacgccccactgatcaccaaccccggcg |
| | ggctccttgagcaggccgtcgcggtcgaggaggccat |
| | cgacaccgccgcggcgaaccagttgatgaacaatgtg |
| | ccccaagcgctgcaacagctggcccagccagcgcagg |
| | gcgtcgtaccttcttccaagctgggtgggctgtggac |
| | ggcggtctcgccgcatctgtcgccgctcagcaacgtc |
| | agttcgatagccaacaaccacatgtcgatgatgggca |
| | cgggtgtgtcgatgaccaacaccttgcactcgatgtt |
| | gaagggcttagctccggcggcggctcaggccgtggaa |
| | accgcggcggaaaacggggtctgggcgatgagctcgc |
| | tgggcagccagctgggttcgtcgctgggttcttcggg |
| | tctgggcgctggggtggccgccaacttgggtcgggcg |
| | gcctcggtcggttcgttgtcggtgccgccagcatggg |
| | ccgcggccaaccaggcggtcaccccggcggcgcgggc |
| | gctgccgctgaccagcctgaccagcgccgcccaaacc |
| | gcccccggacacatgctgggcgggctaccgctggggc |
| | actcggtcaacgccggcagcggtatcaacaatgcgct |
| | gcgggtgccggcacgggcctacgcgataccccgcaca |
| | ccggccgccggagaattcttctcccggccggggctgc |
| | cggtcgagtacctgcaggtgccgtcgccgtcgatggg |
| | ccgcgacatcaaggttcagttccagagcggtgggaac |
| | aactcacctgcggtttatctgctcgacggcctgcgcg |
| | cccaagacgactacaacggctgggatatcaacacccc |
| | ggcgttcgagtggtactaccagtcgggactgtcgata |
| | gtcatgccggtcggcgggcagtccagcttctacagcg |
| | actggtacagcccggcctgcggtaaggctggctgcca |
| | gacttacaagtgggaaaccttcctgaccagcgagctg |
| | ccgcaatggttgtccgccaacagggccgtgaagccca |
| | ccggcagcgctgcaatcggcttgtcgatggccggctc |
| | gtcggcaatgatcttggccgcctaccacccccagcag |
| | ttcatctacgccggctcgctgtcggccctgctggacc |
| | cctctcaggggatggggcctagcctgatcggcctcgc |
| | gatgggtgacgccggcggttacaaggccgcagacatg |
| | tggggtccctcgagtgacccggcatgggagcgcaacg |
| | accctacgcagcagatccccaagctggtcgcaaacaa |
| | cacccggctatgggtttattgcgggaacggcaccccg |
| | aacgagttgggcggtgccaacatacccgccgagttct |
| | tggagaacttcgttcgtagcagcaacctgaagttcca |
| | ggatgcgtacaacgccgcgggcgggcacaacgccgtg |
| | ttcaacttcccgcccaacggcacgcacagctgggagt |
| | actggggcgctcagctcaacgccatgaagggtgacct |
| | gcagagttcgttaggcgccggc |
| ID91 | ID91 |
| | atga ccatcaacta tcaattcggg gacgtcgacg ctcacggcgc catgatccgc |
| | gctcaggccg ggtcgctgga ggccgagcat caggccatca tttctgatgt gttgaccgcg |
| | agtgactttt ggggcggcgc cggttcggcg gcctgccagg ggttcattac ccagctgggc |
| | cgtaacttcc aggtgatcta cgagcaggcc aacgcccacg ggcagaaggt gcaggctgcc |
| | ggcaacaaca tggcacaaac cgacagcgcc gtcggctcca gctgggccgg taccgacgac |
| | atcgattggg acgccatcgc gcaatgcgaa tccggcggca attgggcggc caacaccggt |
| | aacgggttat acggtggtct gcagatcagc caggcgacgt gggattccaa cggtggtgtc |
| | gggtcgccgg cggccgcgag tccccagcaa cagatcgagg tcgcagacaa cattatgaaa |
| | acccaaggcc cgggtgcgtg gccgaaatgt agttcttgta gtcagggaga cgcaccgctg |
| | ggctcgctca cccacatcct gacgttcctc gcggccgaga ctggaggttg ttcggggagc |
| | agggacgatg gatccgtggt ggatttcggg gcgttaccac cggagatcaa ctccgcgagg |
| | atgtacgccg gcccgggttc ggcctcgctg gtggccgccg cgaagatgtg ggacagcgtg |
| | gcgagtgacc tgttttcggc cgcgtcggcg tttcagtcgg tggtctgggg tctgacggtg |
| | gggtcgtgga taggttcgtc ggcgggtctg atggcggcgg cggcctcgcc gtatgtggcg |
| | tggatgagcg tcaccgcggg gcaggcccag ctgaccgccg cccaggtccg ggttgctgcg |
| | gcggcctacg agacagcgta taggctgacg gtgcccccgc cggtgatcgc cgagaaccgt |
| | accgaactga tgacgctgac cgcgaccaac ctcttggggc aaaacacgcc ggcgatcgag |
| | gccaatcagg ccgcatacag ccagatgtgg ggccaagacg cggaggcgat gtatggctac |
| | gccgccacgg cggcgacggc gaccgaggcg ttgctgccgt tcgaggacgc cccactgatc |
| | accaaccccg gcgggctcct tgagcaggcc gtcgcggtcg aggaggccat cgacaccgcc |
| | gcggcgaacc agttgatgaa caatgtgccc caagcgctgc aacagctggc ccagccagcg |
| | cagggcgtcg taccttcttc caagctgggt gggctgtgga cggcggtctc gccgcatctg |
| | tcgccgctca gcaacgtcag ttcgatagcc aacaaccaca tgtcgatgat gggcacgggt |
| | gtgtcgatga ccaacacctt gcactcgatg ttgaagggct tagctccggc ggcggctcag |
| | gccgtggaaa ccgcggcgga aaacggggtc tgggcgatga gctcgctggg cagccagctg |
| | ggttcgtcgc tgggttcttc gggtctgggc gctggggtgg ccgccaactt gggtcgggcg |
| | gcctcggtcg gttcgttgtc ggtgccgcca gcatgggccg cggccaacca ggcggtcacc |
| | ccggcggcgc gggcgctgcc gctgaccagc ctgaccagcg ccgcccaaac cgcccccgga |
| | cacatgctgg gcgggctacc gctggggcac tcggtcaacg ccggcagcgg tatcaacaat |
| | gcgctgcggg tgccggcacg ggcctacgcg ataccccgca caccggccgc cggagaattc |
| | ttctcccggc cggggctgcc ggtcgagtac ctgcaggtgc cgtcgccgtc gatgggccgc |
| | gacatcaagg ttcagttcca gagcggtggg aacaactcac ctgcggttta tctgctcgac |
| | ggcctgcgcg cccaagacga ctacaacggc tgggatatca acaccccggc gttcgagtgg |
| | tactaccagt cgggactgtc gatagtcatg ccggtcggcg ggcagtccag cttctacagc |
| | gactggtaca gcccggcctg cggtaaggct ggctgccaga cttacaagtg ggaaaccttc |
| | ctgaccagcg agctgccgca atggttgtcc gccaacaggg ccgtgaagcc caccggcagc |
| | gctgcaatcg gcttgtcgat ggccggctcg tcggcaatga tcttggccgc ctaccacccc |
| | cagcagttca tctacgccgg ctcgctgtcg gccctgctgg acccctctca ggggatgggg |
| | cctagcctga tcggcctcgc gatgggtgac gccggcggtt acaaggccgc agacatgtgg |
| | ggtccctcga gtgacccggc atgggagcgc aacgacccta cgcagcagat ccccaagctg |
| | gtcgcaaaca acacccggct atgggtttat tgcgggaacg gcaccccgaa cgagttgggc |
| | ggtgccaaca tacccgccga gttcttggag aacttcgttc gtagcagcaa cctgaagttc |
| | caggatgcgt acaacgccgc gggcgggcac aacgccgtgt tcaacttccc gcccaacggc |
| | acgcacagct gggagtactg gggcgctcag ctcaacgcca tgaagggtga cctgcagagt |
| | tcgttaggcg ccggc |
| ID93-1 | ID93-1 and ID93-2 |
| and | atgaccatca actatcaatt cggggacgtc gacgctcacg gcgccatgat ccgcgctcag |
| ID93-2 | gccgggtcgc tggaggccga gcatcaggcc atcatttctg atgtgttgac cgcgagtgac |
| | ttttggggcg gcgccggttc ggcggcctgc caggggttca ttacccagct gggccgtaac |
| | ttccaggtga tctacgagca ggccaacgcc cacgggcaga aggtgcaggc tgccggcaac |
| | aacatggcac aaaccgacag cgccgtcggc tccagctggg ccggtaccca tctcgccaac |
| | ggttcgatgt cggaagtcat gatgtcggaa attgccgggt tgcctatccc tccgattatc |
| | cattacgggg cgattgccta tgcccccagc ggcgcgtcgg gcaaagcgtg gcaccagcgc |
| | acaccggcgc gagcagagca agtcgcacta gaaaagtgcg gtgacaagac ttgcaaagtg |
| | gttagtcgct tcaccaggtg cggcgcggtc gcctacaacg gctcgaaata ccaaggcgga |
| | accggactca cgcgccgcgc ggcagaagac gacgccgtga accgactcga aggcgggcgg |
| | atcgtcaact gggcgtgcaa cgagctcatg acctcgcgtt ttatgacgga tccgcacgcg |
| | atgcgggaca tggcgggccg ttttgaggtg cacgcccaga cggtggagga cgaggctcgc |
| | cggatgtggg cgtccgcgca aaacatctcg ggcgcgggct ggagtggcat ggccgaggcg |
| | acctcgctag acaccatgac ccagatgaat caggcgtttc gcaacatcgt gaacatgctg |
| | cacggggtgc gtgacgggct ggttcgcgac gccaacaact acgaacagca agagcaggcc |
| | tcccagcaga tcctcagcag cgtcgac [symbol] aatttcgccg ttttgccgcc ggaggtgaat |
| | tcggcgcgca tattcgccgg tgcgggcctg ggcccaatgc tggcggcggc gtcggcctgg |
| | gacgggttgg ccgaggagtt gcatgccgcg gcgggctcgt tcgcgtcggt gaccaccggg |
| | ttggcgggcg acgcgtggca tggtccggcg tcgctggcga tgacccgcgc ggccagcccg |
| | tatgtggggt ggttgaacac ggcggcgggt caggccgcgc aggcggccgg ccaggcgcgg |
| | ctagcggcga gcgcgttcga ggcgacgctg gcggccaccg tgtctccagc gatggtcgcg |
| | gccaaccgga cacggctggc gtcgctggtg gcagccaact tgctgggcca gaacgccccg |
| | gcgatcgcgg ccgcggaggc tgaatacgag cagatatggg cccaggacgt ggccgcgatg |
| | ttcggctatc actccgccgc gtcggcggtg gccacgcagc tggcgcctat tcaagagggt |
| | ttgcagcagc agctgcaaaa cgtgctggcc cagttggcta gcgggaacct gggcagcgga |
| | aatgtgggcg tcggcaacat cggcaacgac aacattggca acgcaaacat cggcttcgga |
| | aatcgaggcg acgccaacat cggcatcggg aatatcggcg acagaaacct cggcattggg |
| | aacaccggca attggaatat cggcatcggc atcaccggca acggacaaat cggcttcggc |
| | aagcctgcca accccgacgt cttggtggtg ggcaacggcg gcccgggagt aaccgcgttg |
| | gtcatgggcg gcaccgacag cctactgccg ctgcccaaca tccccttact cgagtacgct |
| | gcgcggttca tcacccccgt gcatcccgga tacaccgcta cgttcctgga aacgccatcg |
| | cagtttttcc cattcaccgg gctgaatagc ctgacctatg acgtctccgt ggcccagggc |
| | gtaacgaatc tgcacaccgc gatcatggcg caactcgcgg cgggaaacga agtcgtcgtc |
| | ttcggcacct cccaaagcgc cacgatagcc accttcgaaa tgcgctatct gcaatccctg |
| | ccagcacacc tgcgtccggg tctcgacgaa ttgtccttta cgttgaccgg caatcccaac |
| | cggcccgacg gtggcattct tacgcgtttt ggcttctcca taccgcagtt gggtttcaca |
| | ttgtccggcg cgacgcccgc cgacgcctac cccaccgtcg attacgcgtt ccagtacgac |
| | ggcgtcaacg acttccccaa atacccgctg aatgtcttcg cgaccgccaa cgcgatcgcg |
| | ggcatccttt tcctgcactc cgggttgatt gcgttgccgc ccgatcttgc ctcgggcgtg |
| | gttcaaccgg tgtcctcacc ggacgtcctg accacctaca tcctgctgcc cagccaagat |
| | ctgccgctgc tggtcccgct gcgtgctatc cccctgctgg gaaacccgct tgccgacctc |
| | atccagccgg acttgcgggt gctcgtcgag ttgggttatg accgcaccgc ccaccaggac |
| | gtgcccagcc cgttcggact gtttccggac gtcgattggg ccgaggtggc cgcggacctg |
| | cagcaaggcg ccgtgcaagg cgtcaacgac gccctgtccg gactggggct gccgccgccg |
| | tggcagccgg cgctaccccg acttttcagt act |
| | [symbol] can encode I or M |
| ID94-1 | ID94-1 and ID94-2 |
| and | ggacgaca tcgattggga cgccatcgcg caatgcgaat ccggcggcaa ttgggcggcc |
| ID94-2 | aacaccggta acgggttata cggtggtctg cagatcagcc aggcgacgtg ggattccaac |
| | ggtggtgtcg ggtcgccggc ggccgcgagt ccccagcaac agatcgaggt cgcagacaac |
| | attatgaaaa cccaaggccc gggtgcgtgg ccgaaatgta gttcttgtag tcagggagac |
| | gcaccgctgg gctcgctcac ccacatcctg acgttcctcg cggccgagac tggaggttgt |
| | tcggggagca gggacgatgg tacccatctc gccaacggtt cgatgtcgga agtcatgatg |
| | tcggaaattg ccgggttgcc tatccctccg attatccatt acggggcgat tgcctatgcc |
| | cccagcggcg cgtcgggcaa agcgtggcac cagcgcacac cggcgcgagc agagcaagtc |
| | gcactagaaa agtgcggtga caagacttgc aaagtggtta gtcgcttcac caggtgcggc |
| | gcggtcgcct acaacggctc gaaataccaa ggcggaaccg gactcacgcg ccgcgcggca |
| | gaagacgacg ccgtgaaccg actcgaaggc gggcggatcg tcaactgggc gtgcaacgag |
| | ctcatgacct cgcgttttat gacggatccg cacgcgatgc gggacatggc gggccgtttt |
| | gaggtgcacg cccagacggt ggaggacgag gctcgccgga tgtgggcgtc cgcgcaaaac |
| | atctcgggcg cgggctggag tggcatggcc gaggcgacct cgctagacac catgacccag |
| | atgaatcagg cgtttcgcaa catcgtgaac atgctgcacg gggtgcgtga cgggctggtt |
| | cgcgacgcca acaactacga acagcaagag caggcctccc agcagatcct cagcagcgtc |
| | gac [symbol] aatt tcgccgtttt gccgccggag gtgaattcgg cgcgcatatt cgccggtgcg |
| | ggcctgggcc caatgctggc ggcggcgtcg gcctgggacg ggttggccga ggagttgcat |
| | gccgcggcgg gctcgttcgc gtcggtgacc accgggttgg cgggcgacgc gtggcatggt |
| | ccggcgtcgc tggcgatgac ccgcgcggcc agcccgtatg tggggtggtt gaacacggcg |
| | gcgggtcagg ccgcgcaggc ggccggccag gcgcggctag cggcgagcgc gttcgaggcg |
| | acgctggcgg ccaccgtgtc tccagcgatg gtcgcggcca accggacacg gctggcgtcg |
| | ctggtggcag ccaacttgct gggccagaac gccccggcga tcgcggccgc ggaggctgaa |
| | tacgagcaga tatgggccca ggacgtggcc gcgatgttcg gctatcactc cgccgcgtcg |
| | gcggtggcca cgcagctggc gcctattcaa gagggtttgc agcagcagct gcaaaacgtg |
| | ctggcccagt tggctagcgg gaacctgggc agcggaaatg tgggcgtcgg caacatcggc |
| | aacgacaaca ttggcaacgc aaacatcggc ttcggaaatc gaggcgacgc caacatcggc |
| | atcgggaata tcggcgacag aaacctcggc attgggaaca ccggcaattg gaatatcggc |
| | atcggcatca ccggcaacgg acaaatcggc ttcggcaagc ctgccaaccc cgacgtcttg |
| | gtggtgggca acggcggccc gggagtaacc gcgttggtca tgggcggcac cgacagccta |
| | ctgccgctgc ccaacatccc cttactcgag tacgctgcgc ggttcatcac ccccgtgcat |
| | cccggataca ccgctacgtt cctggaaacg ccatcgcagt ttttcccatt caccgggctg |
| | aatagcctga cctatgacgt ctccgtggcc cagggcgtaa cgaatctgca caccgcgatc |
| | atggcgcaac tcgcggcggg aaacgaagtc gtcgtcttcg gcacctccca aagcgccacg |
| | atagccacct tcgaaatgcg ctatctgcaa tccctgccag cacacctgcg tccgggtctc |
| | gacgaattgt cctttacgtt gaccggcaat cccaaccggc ccgacggtgg cattcttacg |
| | cgttttggct tctccatacc gcagttgggt ttcacattgt ccggcgcgac gcccgccgac |
| | gcctacccca ccgtcgatta cgcgttccag tacgacggcg tcaacgactt ccccaaatac |
| | ccgctgaatg tcttcgcgac cgccaacgcg atcgcgggca tccttttcct gcactccggg |
| | ttgattgcgt tgccgcccga tcttgcctcg ggcgtggttc aaccggtgtc ctcaccggac |
| | gtcctgacca cctacatcct gctgcccagc caagatctgc cgctgctggt cccgctgcgt |
| | gctatccccc tgctgggaaa cccgcttgcc gacctcatcc agccggactt gcgggtgctc |
| | gtcgagttgg gttatgaccg caccgcccac caggacgtgc ccagcccgtt cggactgttt |
| | ccggacgtcg attgggccga ggtggccgcg gacctgcagc aaggcgccgt gcaaggcgtc |
| | aacgacgccc tgtccggact ggggctgccg ccgccgtggc agccggcgct accccgactt |
| | ttcagtact |
| | [symbol] can encode I or M |
| ID95 | ID95 |
| | gacgaca tcgattggga cgccatcgcg caatgcgaat ccggcggcaa ttgggcggcc |
| | aacaccggta acgggttata cggtggtctg cagatcagcc aggcgacgtg ggattccaac |
| | ggtggtgtcg ggtcgccggc ggccgcgagt ccccagcaac agatcgaggt cgcagacaac |
| | attatgaaaa cccaaggccc gggtgcgtgg ccgaaatgta gttcttgtag tcagggagac |
| | gcaccgctgg gctcgctcac ccacatcctg acgttcctcg cggccgagac tggaggttgt |
| | tcggggagca gggacgatga gctcagtcct tgtgcatatt ttcttgtcta cgaatcaacc |
| | gaaacgaccg agcggcccga gcaccatgaa ttcaagcagg cggcggtgtt gaccgacctg |
| | cccggcgagc tgatgtccgc gctatcgcag gggttgtccc agttcgggat caacataccg |
| | ccggtgccca gcctgaccgg gagcggcgat gccagcacgg gtctaaccgg tcctggcctg |
| | actagtccgg gattgaccag cccgggattg accagcccgg gcctcaccga ccctgccctt |
| | accagtccgg gcctgacgcc aaccctgccc ggatcactcg ccgcgcccgg caccaccctg |
| | gcgccaacgc ccggcgtggg ggccaatccg gcgctcacca accccgcgct gaccagcccg |
| | accggggcga cgccgggatt gaccagcccg acgggtttgg atcccgcgct gggcggcgcc |
| | aacgaaatcc cgattacgac gccggtcgga ttggatcccg gggctgacgg cacctatccg |
| | atcctcggtg atccaacact ggggaccata ccgagcagcc ccgccaccac ctccaccggc |
| | ggcggcggtc tcgtcaacga cgtgatgcag gtggccaacg agttgggcgc cagtcaggct |
| | atcgacctgc taaaaggtgt gctaatgccg tcgatcatgc aggccgtcca gaatggcggc |
| | gcggccgcgc cggcagccag cccgccggtc ccgcccatcc ccgcggccgc ggcggtgcca |
| | ccgacggacc caatcaccgt gccggtcgcc ggtacccatc tcgccaacgg ttcgatgtcg |
| | gaagtcatga tgtcggaaat tgccgggttg cctatccctc cgattatcca ttacggggcg |
| | attgcctatg cccccagcgg cgcgtcgggc aaagcgtggc accagcgcac accggcgcga |
| | gcagagcaag tcgcactaga aaagtgcggt gacaagactt gcaaagtggt tagtcgcttc |
| | accaggtgcg gcgcggtcgc ctacaacggc tcgaaatacc aaggcggaac cggactcacg |
| | cgccgcgcgg cagaagacga cgccgtgaac cgactcgaag gcgggcggat cgtcaactgg |
| | gcgtgcaacg agctcatgac ctcgcgtttt atgacggatc cgcacgcgat gcgggacatg |
| | gcgggccgtt ttgaggtgca cgcccagacg gtggaggacg aggctcgccg gatgtgggcg |
| | tccgcgcaaa acatctcggg cgcgggctgg agtggcatgg ccgaggcgac ctcgctagac |
| | accatgaccc agatgaatca ggcgtttcgc aacatcgtga acatgctgca cggggtgcgt |
| | gacgggctgg ttcgcgacgc caacaactac gaacagcaag agcaggcctc ccagcagatc |
| | ctcagcagcg tcgacatggt cgatgcccac cgcggcggcc acccgacccc gatgagctcg |
| | acgaaggcca cgctgcggct ggccgaggcc accgacagct cgggcaagat caccaagcgc |
| | ggagccgaca agctgatttc caccatcgac gaattcgcca agattgccat cagctcgggc |
| | tgtgccgagc tgatggcctt cgccacgtcg gcggtccgcg acgccgagaa ttccgaggac |
| | gtcctgtccc gggtgcgcaa agagaccggt gtcgagttgc aggcgctgcg tggggaggac |
| | gagtcacggc tgaccttcct ggccgtgcga cgatggtacg ggtggagcgc tgggcgcatc |
| | ctcaacctcg acatcggcgg cggctcgctg gaagtgtcca gtggcgtgga cgaggagccc |
| | gagattgcgt tatcgctgcc cctgggcgcc ggacggttga cccgagagtg gctgcccgac |
| | gatccgccgg gccggcgccg ggtggcgatg ctgcgagact ggctggatgc cgagctggcc |
| | gagcccagtg tgaccgtcct ggaagccggc agccccgacc tggcggtcgc aacgtcgaag |
| | acgtttcgct cgttggcgcg actaaccggt gcggccccat ccatggccgg gccgcgggtg |
| | aagaggaccc taacggcaaa tggtctgcgg caactcatcg cgtttatctc taggatgacg |
| | gcggttgacc gtgcagaact ggaaggggta agcgccgacc gagcgccgca gattgtggcc |
| | ggcgccctgg tggcagaggc gagcatgcga gcactgtcga tagaagcggt ggaaatctgc |
| | ccgtgggcgc tgcgggaagg tctcatcttg cgcaaactcg acagcgaagc cgacggaacc |
| | gccctcatcg agtcttcgtc tgtgcacact tcggtgcgtg ccgtcggagg tcagccagct |
| | gatcggaacg cggccaaccg atcgagaggc agcaaaccaa gtact |
| ID97 | ID97 |
| | atgaccatcaactatcaattcggggacgtcgacgctcac |
| | ggcgccatgatccgcgctcaggccgggtcgctggaggcc |
| | gagcatcaggccatcatttctgatgtgttgaccgcgagt |
| | gacttttggggcggcgccggttcggcggcctgccagggg |
| | ttcattacccagctgggccgtaacttccaggtgatctac |
| | gagcaggccaacgcccacgggcagaaggtgcaggctgcc |
| | ggcaacaacatggcacaaaccgacagcgccgtcggctcc |
| | agctgggccggtaccatgggcgatctggtgagcccgggc |
| | tgcgcggaatacgcggcagccaatcccactgggccggcc |
| | tcggtgcagggaatgtcgcaggacccggtcgcggtggcg |
| | gcctcgaacaatccggagttgacaacgctgacggctgca |
| | ctgtcgggccagctcaatccgcaagtaaacctggtggac |
| | accctcaacagcggtcagtacacggtgttcgcaccgacc |
| | aacgcggcatttagcaagctgccggcatccacgatcgac |
| | gagctcaagaccaattcgtcactgctgaccagcatcctg |
| | acctaccacgtagtggccggccaaaccagcccggccaac |
| | gtcgtcggcacccgtcagaccctccagggcgccagcgtg |
| | acggtgaccggtcagggtaacagcctcaaggtcggtaac |
| | gccgacgtcgtctgtggtggggtgtctaccgccaacgcg |
| | acggtgtacatgattgacagcgtgctaatgcctccggcg |
| | ggatccgtggtggatttcggggcgttaccaccggagatc |
| | aactccgcgaggatgtacgccggcccgggttcggcctcg |
| | ctggtggccgccgcgaagatgtgggacagcgtggcgagt |
| | gacctgttttcggccgcgtcggcgtttcagtcggtggtc |
| | tggggtctgacggtggggtcgtggataggttcgtcggcg |
| | ggtctgatggcggcggcggcctcgccgtatgtggcgtgg |
| | atgagcgtcaccgcggggcaggcccagctgaccgccgcc |
| | caggtccgggttgctgcggcggcctacgagacagcgtat |
| | aggctgacggtgcccccgccggtgatcgccgagaaccgt |
| | accgaactgatgacgctgaccgcgaccaacctcttgggg |
| | caaaacacgccggcgatcgaggccaatcaggccgcatac |
| | agccagatgtggggccaagacgcggaggcgatgtatggc |
| | tacgccgccacggcggcgacggcgaccgaggcgttgctg |
| | ccgttcgaggacgccccactgatcaccaaccccggcggg |
| | ctccttgagcaggccgtcgcggtcgaggaggccatcgac |
| | accgccgcggcgaaccagttgatgaacaatgtgccccaa |
| | gcgctgcaacagctggcccagccagcgcagggcgtcgta |
| | ccttcttccaagctgggtgggctgtggacggcggtctcg |
| | ccgcatctgtcgccgctcagcaacgtcagttcgatagcc |
| | aacaaccacatgtcgatgatgggcacgggtgtgtcgatg |
| | accaacaccttgcactcgatgttgaagggcttagctccg |
| | gcggcggctcaggccgtggaaaccgcggcggaaaacggg |
| | gtctgggcgatgagctcgctgggcagccagctgggttcg |
| | tcgctgggttcttcgggtctgggcgctggggtggccgcc |
| | aacttgggtcgggcggcctcggtcggttcgttgtcggtg |
| | ccgccagcatgggccgcggccaaccaggcggtcaccccg |
| | gcggcgcgggcgctgccgctgaccagcctgaccagcgcc |
| | gcccaaaccgcccccggacacatgctgggcgggctaccg |
| | ctggggcactcggtcaacgccggcagcggtatcaacaat |
| | gcgctgcgggtgccggcacgggcctacgcgataccccgc |
| | acaccggccgccggagaattcttctcccggccggggctg |
| | ccggtcgagtacctgcaggtgccgtcgccgtcgatgggc |
| | cgcgacatcaaggttcagttccagagcggtgggaacaac |
| | tcacctgcggtttatctgctcgacggcctgcgcgcccaa |
| | gacgactacaacggctgggatatcaacaccccggcgttc |
| | gagtggtactaccagtcgggactgtcgatagtcatgccg |
| | gtcggcgggcagtccagcttctacagcgactggtacagc |
| | ccggcctgcggtaaggctggctgccagacttacaagtgg |
| | gaaaccttcctgaccagcgagctgccgcaatggttgtcc |
| | gccaacagggccgtgaagcccaccggcagcgctgcaatc |
| | ggcttgtcgatggccggctcgtcggcaatgatcttggcc |
| | gcctaccacccccagcagttcatctacgccggctcgctg |
| | tcggccctgctggacccctctcaggggatggggcctagc |
| | ctgatcggcctcgcgatgggtgacgccggcggttacaag |
| | gccgcagacatgtggggtccctcgagtgacccggcatgg |
| | gagcgcaacgaccctacgcagcagatccccaagctggtc |
| | gcaaacaacacccggctatgggtttattgcgggaacggc |
| | accccgaacgagttgggcggtgccaacatacccgccgag |
| | ttcttggagaacttcgttcgtagcagcaacctgaagttc |
| | caggatgcgtacaacgccgcgggcgggcacaacgccgtg |
| | ttcaacttcccgcccaacggcacgcacagctgggagtac |
| | tggggcgctcagctcaacgccatgaagggtgacctgcag |
| | agttcgttaggcgccggc |
| ID114 | ID114 |
| | ggtaccc atctcgccaa cggttcgatg tcggaagtca tgatgtcgga aattgccggg |
| | ttgcctatcc ctccgattat ccattacggg gcgattgcct atgcccccag cggcgcgtcg |
| | ggcaaagcgt ggcaccagcg cacaccggcg cgagcagagc aagtcgcact agaaaagtgc |
| | ggtgacaaga cttgcaaagt ggttagtcgc ttcaccaggt gcggcgcggt cgcctacaac |
| | ggctcgaaat accaaggcgg aaccggactc acgcgccgcg cggcagaaga cgacgccgtg |
| | aaccgactcg aaggcgggcg gatcgtcaac tgggcgtgca acgagctcat gacctcgcgt |
| | tttatgacgg atccgcacgc gatgcgggac atggcgggcc gttttgaggt gcacgcccag |
| | acggtggagg acgaggctcg ccggatgtgg gcgtccgcgc aaaacatctc gggcgcgggc |
| | tggagtggca tggccgaggc gacctcgcta gacaccatga cccagatgaa tcaggcgttt |
| | cgcaacatcg tgaacatgct gcacggggtg cgtgacgggc tggttcgcga cgccaacaac |
| | tacgaacagc aagagcaggc ctcccagcag atcctcagca gcgtcgacat caatttcgcc |
| | gttttgccgc cggaggtgaa ttcggcgcgc atattcgccg gtgcgggcct gggcccaatg |
| | ctggcggcgg cgtcggcctg ggacgggttg gccgaggagt tgcatgccgc ggcgggctcg |
| | ttcgcgtcgg tgaccaccgg gttggcgggc gacgcgtggc atggtccggc gtcgctggcg |
| | atgacccgcg cggccagccc gtatgtgggg tggttgaaca cggcggcggg tcaggccgcg |
| | caggcggccg gccaggcgcg gctagcggcg agcgcgttcg aggcgacgct ggcggccacc |
| | gtgtctccag cgatggtcgc ggccaaccgg acacggctgg cgtcgctggt ggcagccaac |
| | ttgctgggcc agaacgcccc ggcgatcgcg gccgcggagg ctgaatacga gcagatatgg |
| | gcccaggacg tggccgcgat gttcggctat cactccgccg cgtcggcggt ggccacgcag |
| | ctggcgccta ttcaagaggg tttgcagcag cagctgcaaa acgtgctggc ccagttggct |
| | agcgggaacc tgggcagcgg aaatgtgggc gtcggcaaca tcggcaacga caacattggc |
| | aacgcaaaca tcggcttcgg aaatcgaggc gacgccaaca tcggcatcgg gaatatcggc |
| | gacagaaacc tcggcattgg gaacaccggc aattggaata tcggcatcgg catcaccggc |
| | aacggacaaa tcggcttcgg caagcctgcc aaccccgacg tcttggtggt gggcaacggc |
| | ggcccgggag taaccgcgtt ggtcatgggc ggcaccgaca gcctactgcc gctgcccaac |
| | atccccttac tcgagtacgc tgcgcggttc atcacccccg tgcatcccgg atacaccgct |
| | acgttcctgg aaacgccatc gcagifittc ccattcaccg ggctgaatag cctgacctat |
| | gacgtctccg tggcccaggg cgtaacgaat ctgcacaccg cgatcatggc gcaactcgcg |
| | gcgggaaacg aagtcgtcgt cttcggcacc tcccaaagcg ccacgatagc caccttcgaa |
| | atgcgctatc tgcaatccct gccagcacac ctgcgtccgg gtctcgacga attgtccttt |
| | acgttgaccg gcaatcccaa ccggcccgac ggtggcattc ttacgcgttt tggcttctcc |
| | ataccgcagt tgggtttcac attgtccggc gcgacgcccg ccgacgccta ccccaccgtc |
| | gattacgcgt tccagtacga cggcgtcaac gacttcccca aatacccgct gaatgtcttc |
| | gcgaccgcca acgcgatcgc gggcatcctt ttcctgcact ccgggttgat tgcgttgccg |
| | cccgatcttg cctcgggcgt ggttcaaccg gtgtcctcac cggacgtcct gaccacctac |
| | atcctgctgc ccagccaaga tctgccgctg ctggtcccgc tgcgtgctat ccccctgctg |
| | ggaaacccgc ttgccgacct catccagccg gacttgcggg tgctcgtcga gttgggttat |
| | gaccgcaccg cccaccagga cgtgcccagc ccgttcggac tgtttccgga cgtcgattgg |
| | gccgaggtgg ccgcggacct gcagcaaggc gccgtgcaag gcgtcaacga cgccctgtcc |
| | ggactggggc tgccgccgcc gtggcagccg gcgctacccc gacttttcag tactttctcc |
| | cggccggggc tgccggtcga gtacctgcag gtgccgtcgc cgtcgatggg ccgcgacatc |
| | aaggttcagt tccagagcgg tgggaacaac tcacctgcgg tttatctgct cgacggcctg |
| | cgcgcccaag acgactacaa cggctgggat atcaacaccc cggcgttcga gtggtactac |
| | cagtcgggac tgtcgatagt catgccggtc ggcgggcagt ccagcttcta cagcgactgg |
| | tacagcccgg cctgcggtaa ggctggctgc cagacttaca agtgggaaac cttcctgacc |
| | agcgagctgc cgcaatggtt gtccgccaac agggccgtga agcccaccgg cagcgctgca |
| | atcggcttgt cgatggccgg ctcgtcggca atgatcttgg ccgcctacca cccccagcag |
| | ttcatctacg ccggctcgct gtcggccctg ctggacccct ctcaggggat ggggcctagc |
| | ctgatcggcc tcgcgatggg tgacgccggc ggttacaagg ccgcagacat gtggggtccc |
| | tcgagtgacc cggcatggga gcgcaacgac cctacgcagc agatccccaa gctggtcgca |
| | aacaacaccc ggctatgggt ttattgcggg aacggcaccc cgaacgagtt gggcggtgcc |
| | aacatacccg ccgagttctt ggagaacttc gttcgtagca gcaacctgaa gttccaggat |
| | gcgtacaacg ccgcgggcgg gcacaacgcc gtgttcaact tcccgcccaa cggcacgcac |
| | agctgggagt actggggcgc tcagctcaac gccatgaagg gtgacctgca gagttcgtta |
| | ggcgccggc |
| ID120-1 | ID120-1 and ID120-2 |
| and | gacgaca tcgattggga cgccatcgcg caatgcgaat ccggcggcaa ttgggcggcc |
| ID120-2 | aacaccggta acgggttata cggtggtctg cagatcagcc aggcgacgtg ggattccaac |
| | ggtggtgtcg ggtcgccggc ggccgcgagt ccccagcaac agatcgaggt cgcagacaac |
| | attatgaaaa cccaaggccc gggtgcgtgg ccgaaatgta gttcttgtag tcagggagac |
| | gcaccgctgg gctcgctcac ccacatcctg acgttcctcg cggccgagac tggaggttgt |
| | tcggggagca gggacgatga gctcagtcct tgtgcatatt ttcttgtcta cgaatcaacc |
| | gaaacgaccg agcggcccga gcaccatgaa ttcaagcagg cggcggtgtt gaccgacctg |
| | cccggcgagc tgatgtccgc gctatcgcag gggttgtccc agttcgggat caacataccg |
| | ccggtgccca gcctgaccgg gagcggcgat gccagcacgg gtctaaccgg tcctggcctg |
| | actagtccgg gattgaccag cccgggattg accagcccgg gcctcaccga ccctgccctt |
| | accagtccgg gcctgacgcc aaccctgccc ggatcactcg ccgcgcccgg caccaccctg |
| | gcgccaacgc ccggcgtggg ggccaatccg gcgctcacca accccgcgct gaccagcccg |
| | accggggcga cgccgggatt gaccagcccg acgggtttgg atcccgcgct gggcggcgcc |
| | aacgaaatcc cgattacgac gccggtcgga ttggatcccg gggctgacgg cacctatccg |
| | atcctcggtg atccaacact ggggaccata ccgagcagcc ccgccaccac ctccaccggc |
| | ggcggcggtc tcgtcaacga cgtgatgcag gtggccaacg agttgggcgc cagtcaggct |
| | atcgacctgc taaaaggtgt gctaatgccg tcgatcatgc aggccgtcca gaatggcggc |
| | gcggccgcgc cggcagccag cccgccggtc ccgcccatcc ccgcggccgc ggcggtgcca |
| | ccgacggacc caatcaccgt gccggtcgcc ggtacccatc tcgccaacgg ttcgatgtcg |
| | gaagtcatga tgtcggaaat tgccgggttg cctatccctc cgattatcca ttacggggcg |
| | attgcctatg cccccagcgg cgcgtcgggc aaagcgtggc accagcgcac accggcgcga |
| | gcagagcaag tcgcactaga aaagtgcggt gacaagactt gcaaagtggt tagtcgcttc |
| | accaggtgcg gcgcggtcgc ctacaacggc tcgaaatacc aaggcggaac cggactcacg |
| | cgccgcgcgg cagaagacga cgccgtgaac cgactcgaag gcgggcggat cgtcaactgg |
| | gcgtgcaacg agctcatgac ctcgcgtttt atgacggatc cgcacgcgat gcgggacatg |
| | gcgggccgtt ttgaggtgca cgcccagacg gtggaggacg aggctcgccg gatgtgggcg |
| | tccgcgcaaa acatctcggg cgcgggctgg agtggcatgg ccgaggcgac ctcgctagac |
| | accatgaccc agatgaatca ggcgtttcgc aacatcgtga acatgctgca cggggtgcgt |
| | gacgggctgg ttcgcgacgc caacaactac gaacagcaag agcaggcctc ccagcagatc |
| | ctcagcagcg tcgac [symbol] aa tttcgccgtt ttgccgccgg aggtgaattc ggcgcgcata |
| | ttcgccggtg cgggcctggg cccaatgctg gcggcggcgt cggcctggga cgggttggcc |
| | gaggagttgc atgccgcggc gggctcgttc gcgtcggtga ccaccgggtt ggcgggcgac |
| | gcgtggcatg gtccggcgtc gctggcgatg acccgcgcgg ccagcccgta tgtggggtgg |
| | ttgaacacgg cggcgggtca ggccgcgcag gcggccggcc aggcgcggct agcggcgagc |
| | gcgttcgagg cgacgctggc ggccaccgtg tctccagcga tggtcgcggc caaccggaca |
| | cggctggcgt cgctggtggc agccaacttg ctgggccaga acgccccggc gatcgcggcc |
| | gcggaggctg aatacgagca gatatgggcc caggacgtgg ccgcgatgtt cggctatcac |
| | tccgccgcgt cggcggtggc cacgcagctg gcgcctattc aagagggttt gcagcagcag |
| | ctgcaaaacg tgctggccca gttggctagc gggaacctgg gcagcggaaa tgtgggcgtc |
| | ggcaacatcg gcaacgacaa cattggcaac gcaaacatcg gcttcggaaa tcgaggcgac |
| | gccaacatcg gcatcgggaa tatcggcgac agaaacctcg gcattgggaa caccggcaat |
| | tggaatatcg gcatcggcat caccggcaac ggacaaatcg gcttcggcaa gcctgccaac |
| | cccgacgtct tggtggtggg caacggcggc ccgggagtaa ccgcgttggt catgggcggc |
| | accgacagcc tactgccgct gcccaacatc cccttactcg agtacgctgc gcggttcatc |
| | acccccgtgc atcccggata caccgctacg ttcctggaaa cgccatcgca gtttttccca |
| | ttcaccgggc tgaatagcct gacctatgac gtctccgtgg cccagggcgt aacgaatctg |
| | cacaccgcga tcatggcgca actcgcggcg ggaaacgaag tcgtcgtctt cggcacctcc |
| | caaagcgcca cgatagccac cttcgaaatg cgctatctgc aatccctgcc agcacacctg |
| | cgtccgggtc tcgacgaatt gtcctttacg ttgaccggca atcccaaccg gcccgacggt |
| | ggcattctta cgcgttttgg cttctccata ccgcagttgg gtttcacatt gtccggcgcg |
| | acgcccgccg acgcctaccc caccgtcgat tacgcgttcc agtacgacgg cgtcaacgac |
| | ttccccaaat acccgctgaa tgtcttcgcg accgccaacg cgatcgcggg catccttttc |
| | ctgcactccg ggttgattgc gttgccgccc gatcttgcct cgggcgtggt tcaaccggtg |
| | tcctcaccgg acgtcctgac cacctacatc ctgctgccca gccaagatct gccgctgctg |
| | gtcccgctgc gtgctatccc cctgctggga aacccgcttg ccgacctcat ccagccggac |
| | ttgcgggtgc tcgtcgagtt gggttatgac cgcaccgccc accaggacgt gcccagcccg |
| | ttcggactgt ttccggacgt cgattgggcc gaggtggccg cggacctgca gcaaggcgcc |
| | gtgcaaggcg tcaacgacgc cctgtccgga ctggggctgc cgccgccgtg gcagccggcg |
| | ctaccccgac ttttcagtac t |
| | [symbol] can encode I or M |
| ID125-1 | ID125-1 and ID125-2 |
| and | atgaccatca actatcaatt cggggacgtc gacgctcacg gcgccatgat ccgcgctcag |
| ID125-2 | gccgggtcgc tggaggccga gcatcaggcc atcatttctg atgtgttgac cgcgagtgac |
| | ttttggggcg gcgccggttc ggcggcctgc caggggttca ttacccagct gggccgtaac |
| | ttccaggtga tctacgagca ggccaacgcc cacgggcaga aggtgcaggc tgccggcaac |
| | aacatggcac aaaccgacag cgccgtcggc tccagctggg ccggtaccca tctcgccaac |
| | ggttcgatgt cggaagtcat gatgtcggaa attgccgggt tgcctatccc tccgattatc |
| | cattacgggg cgattgccta tgcccccagc ggcgcgtcgg gcaaagcgtg gcaccagcgc |
| | acaccggcgc gagcagagca agtcgcacta gaaaagtgcg gtgacaagac ttgcaaagtg |
| | gttagtcgct tcaccaggtg cggcgcggtc gcctacaacg gctcgaaata ccaaggcgga |
| | accggactca cgcgccgcgc ggcagaagac gacgccgtga accgactcga aggcgggcgg |
| | atcgtcaact gggcgtgcaa cgagctcatg acctcgcgtt ttatgacgga tccgcacgcg |
| | atgcgggaca tggcgggccg ttttgaggtg cacgcccaga cggtggagga cgaggctcgc |
| | cggatgtggg cgtccgcgca aaacatctcg ggcgcgggct ggagtggcat ggccgaggcg |
| | acctcgctag acaccatgac ccagatgaat caggcgtttc gcaacatcgt gaacatgctg |
| | cacggggtgc gtgacgggct ggttcgcgac gccaacaact acgaacagca agagcaggcc |
| | tcccagcaga tcctcagcag cgtcgac [symbol] aatttcgccg ttttgccgcc ggaggtgaat |
| | tcggcgcgca tattcgccgg tgcgggcctg ggcccaatgc tggcggcggc gtcggcctgg |
| | gacgggttgg ccgaggagtt gcatgccgcg gcgggctcgt tcgcgtcggt gaccaccggg |
| | ttggcgggcg acgcgtggca tggtccggcg tcgctggcga tgacccgcgc ggccagcccg |
| | tatgtggggt ggttgaacac ggcggcgggt caggccgcgc aggcggccgg ccaggcgcgg |
| | ctagcggcga gcgcgttcga ggcgacgctg gcggccaccg tgtctccagc gatggtcgcg |
| | gccaaccgga cacggctggc gtcgctggtg gcagccaact tgctgggcca gaacgccccg |
| | gcgatcgcgg ccgcggaggc tgaatacgag cagatatggg cccaggacgt ggccgcgatg |
| | ttcggctatc actccgccgc gtcggcggtg gccacgcagc tggcgcctat tcaagagggt |
| | ttgcagcagc agctgcaaaa cgtgctggcc cagttggcta gcgggaacct gggcagcgga |
| | aatgtgggcg tcggcaacat cggcaacgac aacattggca acgcaaacat cggcttcgga |
| | aatcgaggcg acgccaacat cggcatcggg aatatcggcg acagaaacct cggcattggg |
| | aacaccggca attggaatat cggcatcggc atcaccggca acggacaaat cggcttcggc |
| | aagcctgcca accccgacgt cttggtggtg ggcaacggcg gcccgggagt aaccgcgttg |
| | gtcatgggcg gcaccgacag cctactgccg ctgcccaaca tccccttact cgagtacgct |
| | gcgcggttca tcacccccgt gcatcccgga tacaccgcta cgttcctgga aacgccatcg |
| | cagtttttcc cattcaccgg gctgaatagc ctgacctatg acgtctccgt ggcccagggc |
| | gtaacgaatc tgcacaccgc gatcatggcg caactcgcgg cgggaaacga agtcgtcgtc |
| | ttcggcacct cccaaagcgc cacgatagcc accttcgaaa tgcgctatct gcaatccctg |
| | ccagcacacc tgcgtccggg tctcgacgaa ttgtccttta cgttgaccgg caatcccaac |
| | cggcccgacg gtggcattct tacgcgtttt ggcttctcca taccgcagtt gggtttcaca |
| | ttgtccggcg cgacgcccgc cgacgcctac cccaccgtcg attacgcgtt ccagtacgac |
| | ggcgtcaacg acttccccaa atacccgctg aatgtcttcg cgaccgccaa cgcgatcgcg |
| | ggcatccttt tcctgcactc cgggttgatt gcgttgccgc ccgatcttgc ctcgggcgtg |
| | gttcaaccgg tgtcctcacc ggacgtcctg accacctaca tcctgctgcc cagccaagat |
| | ctgccgctgc tggtcccgct gcgtgctatc cccctgctgg gaaacccgct tgccgacctc |
| | atccagccgg acttgcgggt gctcgtcgag ttgggttatg accgcaccgc ccaccaggac |
| | gtgcccagcc cgttcggact gtttccggac gtcgattggg ccgaggtggc cgcggacctg |
| | cagcaaggcg ccgtgcaagg cgtcaacgac gccctgtccg gactggggct gccgccgccg |
| | tggcagccgg cgctaccccg acttttcagt actttctccc ggccggggct gccggtcgag |
| | tacctgcagg tgccgtcgcc gtcgatgggc cgcgacatca aggttcagtt ccagagcggt |
| | gggaacaact cacctgcggt ttatctgctc gacggcctgc gcgcccaaga cgactacaac |
| | ggctgggata tcaacacccc ggcgttcgag tggtactacc agtcgggact gtcgatagtc |
| | atgccggtcg gcgggcagtc cagcttctac agcgactggt acagcccggc ctgcggtaag |
| | gctggctgcc agacttacaa gtgggaaacc ttcctgacca gcgagctgcc gcaatggttg |
| | tccgccaaca gggccgtgaa gcccaccggc agcgctgcaa tcggcttgtc gatggccggc |
| | tcgtcggcaa tgatcttggc cgcctaccac ccccagcagt tcatctacgc cggctcgctg |
| | tcggccctgc tggacccctc tcaggggatg gggcctagcc tgatcggcct cgcgatgggt |
| | gacgccggcg gttacaaggc cgcagacatg tggggtccct cgagtgaccc ggcatgggag |
| | cgcaacgacc ctacgcagca gatccccaag ctggtcgcaa acaacacccg gctatgggtt |
| | tattgcggga acggcacccc gaacgagttg ggcggtgcca acatacccgc cgagttcttg |
| | gagaacttcg ttcgtagcag caacctgaag ttccaggatg cgtacaacgc cgcgggcggg |
| | cacaacgccg tgttcaactt cccgcccaac ggcacgcaca gctgggagta ctggggcgct |
| | cagctcaacg ccatgaaggg tgacctgcag agttcgttag gcgccggc |
| | [symbol] can encode I or M |

Prophylactic and Therapeutic Compositions

In another aspect, the present disclosure concerns formulations of one or more of the polynucleotide, polypeptide or other compositions disclosed herein in pharmaceutically-acceptable or physiologically-acceptable solutions for administration to a cell or a subject, either alone, or in combination with one or more other modalities of therapy. Such pharmaceutical compositions can be used for prophylactic or therapeutic embodiments. The formulations can be further use vaccines when formulated with a suitable immunostimulant/adjuvant system.

It will also be understood that, if desired, the compositions of the disclosure may be administered in combination with other agents as well, such as, e.g., other proteins or polypeptides or various pharmaceutically-active agents. There is virtually no limit to other components that may also be included, provided that the additional agents do not cause a significant adverse effect upon the objectives according to the disclosure.

In certain embodiments the compositions of the disclosure are formulated in combination with one or more immunostimulants. An immunostimulant may be any substance that enhances or potentiates an immune response (antibody and/or cell-mediated) to an exogenous antigen. Examples of immunostimulants include adjuvants, biodegradable microspheres (e.g., polylactic galactide) and liposomes (into which the compound is incorporated; see, e.g., Fullerton, U.S. Pat. No. 4,235,877). Vaccine preparation is generally described in, for example, Powell & Newman, eds., Vaccine Design (the subunit and adjuvant approach) (1995).

Any of a variety of immunostimulants may be employed in the compositions of this disclosure. For example, an adjuvant may be included. Many adjuvants contain a substance designed to protect the antigen from rapid catabolism, such as aluminum hydroxide or mineral oil, and a stimulator of immune responses, such as lipid A (natural or synthetic), Bortadella pertussis or Mycobacterium species or Mycobacterium derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 and derivatives thereof (SmithKline Beecham, Philadelphia, Pa.); CWS, TDM, Leif, aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

Other illustrative adjuvants useful in the context of the disclosure include Toll-like receptor agonists, such as TLR1, TLR2, TLR3, TLR4, TLR5, TLR6, TLR7, TLR8, TLR7/8, TLR9 agonists, and the like. Still other illustrative adjuvants include imiquimod, gardiquimod, resiquimod, and related compounds.

Certain exemplary compositions employ adjuvant systems designed to induce an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a compositions as provided herein, a patient may support an immune response that includes Th1- and Th2-type responses. Within an exemplary embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mossman & Coffman, Ann. Rev. Immunol. 7:145-173 (1989).

Certain adjuvants for use in eliciting a predominantly Th1-type response include, for example, a combination of monophosphoryl lipid A, for example 3-de-O-acylated monophosphoryl lipid A (3D-MPLTM), together with an aluminum salt (U.S. Pat. Nos. 4,436,727; 4,877,611; 4,866,034; and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352 (1996). Another illustrative adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins. Other illustrative formulations include more than one saponin in the adjuvant combinations of the present disclosure, for example combinations of at least two of the following group comprising QS21, QS7, Quil A, 0-escin, or digitonin.

In other embodiments, the adjuvant is a glucopyranosyl lipid A (GLA) adjuvant, as described in U.S. Patent Application Publication No. 2008/0131466, the disclosure of which is incorporated herein by reference in its entirety.

In a particular embodiment, the adjuvant system includes the combination of a monophosphoryl lipid A and a saponin derivative, such as the combination of QS21 and 3D-MPLTM. adjuvant, as described in WO 94/00153, or a less reactogenic composition where the QS21 is quenched with cholesterol, as described in WO 96/33739. Other formulations comprise an oil-in-water emulsion and tocopherol. Another adjuvant formulation employing QS21, 3D-MPLTM adjuvant and tocopherol in an oil-in-water emulsion is described in WO 95/17210.

Another enhanced adjuvant system involves the combination of a CpG-containing oligonucleotide and a saponin derivative as disclosed in WO 00/09159. Other illustrative adjuvants include Montanide ISA 720 (Seppic, France), SAF (Chiron, Calif., United States), ISCOMS (CSL), MF-59 (Chiron), the SBAS series of adjuvants (e.g., SBAS-2, AS2′, AS2,″ SBAS-4, or SBAS6, available from SmithKline Beecham, Rixensart, Belgium), Detox, RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1.

Compositions of the disclosure may also, or alternatively, comprise T cells specific for a Mycobacterium antigen. Such cells may generally be prepared in vitro or ex vivo, using standard procedures. For example, T cells may be isolated from bone marrow, peripheral blood, or a fraction of bone marrow or peripheral blood of a patient. Alternatively, T cells may be derived from related or unrelated humans, non-human mammals, cell lines or cultures.

T cells may be stimulated with a polypeptide of the disclosure, polynucleotide encoding such a polypeptide, and/or an antigen presenting cell (APC) that expresses such a polypeptide. Such stimulation is performed under conditions and for a time sufficient to permit the generation of T cells that are specific for the polypeptide. In some embodimetns, the polypeptide or polynucleotide is present within a delivery vehicle, such as a microsphere, to facilitate the generation of specific T cells.

T cells are considered to be specific for a polypeptide of the disclosure if the T cells specifically proliferate, secrete cytokines or kill target cells coated with the polypeptide or expressing a gene encoding the polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070 (1994)). Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a polypeptide of the disclosure (100 ng/ml-100 μg/ml, or even 200 ng/ml-25 μg/ml) for 3-7 days can result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1 (1998)). T cells that have been activated in response to a polypeptide, polynucleotide or polypeptide-expressing APC may be CD4+ and/or CD8+. Protein-specific T cells may be expanded using standard techniques. Within some embodiments, the T cells are derived from a patient, a related donor or an unrelated donor, and are administered to the patient following stimulation and expansion.

In the pharmaceutical compositions of the disclosure, formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intramuscular administration and formulation.

In certain applications, the pharmaceutical compositions disclosed herein may be delivered via oral administration to a subject. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain circumstances it may be desirable to deliver the pharmaceutical compositions disclosed herein parenterally, intravenously, intramuscularly, intranasally, subcutaneously, intrvaginally, rectally, or even intraperitoneally as described, for example, in U.S. Pat. Nos. 5,543,158; 5,641,515 and 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In some embodiments, it may be desirable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution can be be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active compounds in the required amount in the appropriate solvent with the various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, exemplary methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like. Upon formulation, solutions may be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

In certain embodiments, the pharmaceutical compositions may be delivered by intranasal sprays, inhalation, and/or other aerosol delivery vehicles. Methods for delivering genes, polynucleotides, and peptide compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

In certain embodiments, the delivery may occur by use of liposomes, nanocapsules, microparticles, microspheres, lipid particles, vesicles, and the like, for the introduction of the compositions of the present disclosure into suitable host cells. In particular, the compositions of the present disclosure may be formulated for delivery either encapsulated in a lipid particle, a liposome, a vesicle, a nanosphere, a nanoparticle or the like. The formulation and use of such delivery vehicles can be carried out using known and conventional techniques.

Methods of Use

The inventors have found that certain Mycobacterial antigens are capable of eliciting a strong central memory T cell response, and that certain Mycobacterial antigens are capable of eliciting a strong effector memory T cell response. Such dual functionality is of T cell phenotypes contained in a single composition could be tremendously beneficial in improving the efficacy of both prophylactic or therapeutic compositions for preventing or treating secondary TB or a primary or secondary NTM infection. Thus, provided herein are fusion polypeptides comprising at least two Mycobacterial antigens, wherein one Mycobacterial antigen is a strong central memory T cell activator, and wherein one Mycobacterial antigen is a strong effector memory T cell activator. Exemplary fusion polypeptides are provided in Table 2.

A strong central memory T cell activator response is elicited when the FDS of the subject is less than or equal to about 1.0, 0.9, 0.8, 0.7, 0.6, 0.5, 0.4, 0.3, 0.25, 0.2, 0.125, 0.1, or even about 0.0625 within 300 days after a single immunization.

A strong effector memory T cell activator response is elicited when the FDS of the subject is greater than or equal to about 3.0, 4, 5, 6, 7, 8, 9, 10, 16, or even about 32 after one or more immunizations.

Several uses for the fusion polypeptides (and compositions comprising the fusion polypeptides, e.g., pharmaceutical compositions) are provided herein.

In some embodiments, provided herein is a method of activating a strong Mycobacterial central memory T cell response and a strong Mycobacterial effector memory T cell response in a subject comprising administering to a subject an effective amount of any one of the fusion polypeptides, or pharmaceutical compositions comprising the fusion polypeptides provided herein. In some embodiments, the subject is Quantiferon positive. In some embodiments, the subject is Quantiferon negative.

In some embodiments, provided herein is a method of treating secondary tuberculosis infection (e.g., reactivation of a latent Mtb infection), comprising administering to a subject an effective amount of any one of the fusion polypeptides, or pharmaceutical compositions comprising the fusion polypeptides provided herein. In some embodiments, the method is for treating reactivation of a latent Mtb infection. In some embodiments, the subject is Quantiferon positive. In some embodiments, the subject is Quantiferon negative. In some embodiments, the subject is undergoing a first reactivation. In some embodiments, the subject is undergoing a third, fourth, or even fifth instance of reactivation.

In some embodiments, provided herein is a method of preventing secondary tuberculosis infection (e.g., preventing reactivation of a latent Mtb infection) in a subject, comprising administering to a subject an effective amount of any one of the fusion polypeptides, or pharmaceutical compositions comprising the fusion polypeptides provided herein. In some embodiments, the method is for preventing reactivation of a latent Mtb infection. In some embodiments, the subject is Quantiferon positive. In some embodiments, the subject is Quantiferon negative. In some embodiments, the subject is undergoing a first reactivation. In some embodiments, the subject is undergoing a third, fourth, or even fifth instance of reactivation.

In some embodiments, provided herein is a method of treating secondary tuberculosis infection (e.g., a second infection with a Mtb) in a subject, comprising administering to a subject an effective amount of any one of the fusion polypeptides, or pharmaceutical compositions comprising the fusion polypeptides provided herein. In some embodiments, the method is for preventing second infection with a Mtb, wherein the first infection was with a Mtb of a different strain (a different clinical isolate). In some embodiments, the second infection is with a multidrug resistant (MDR) Mtb strain. In some embodiments, the subject is Quantiferon positive. In some embodiments, the subject is Quantiferon negative.

In some embodiments, provided herein is a method of preventing secondary tuberculosis infection (preventing a second infection with a Mtb) in a subject, comprising administering to a subject an effective amount of any one of the fusion polypeptides, or pharmaceutical compositions comprising the fusion polypeptides provided herein. In some embodiments, the method is for preventing second infection with aMtb, wherein the first infection was with aMtb of a different strain (a different clinical isolate). In some embodiments, the second infection is with a multidrug resistant (MDR) Mtb strain. In some embodiments, the subject is Quantiferon positive. In some embodiments, the subject is Quantiferon negative.

In some embodiments, provided herein is a method of treating a nontuberculous Mycobacterium (NTM) infection in a subject, comprising administering to a subject an effective amount of any one of the fusion polypeptides, or pharmaceutical compositions comprising the fusion polypeptides provided herein. In some embodiments, the subject is Quantiferon positive. In some embodiments, the subject is Quantiferon negative. In any of these embodiments, the NTM infection can be the primary instance of a NTM infection or the second instance of a NTM infection (e.g., a secondary infection). The NTM can be any one of the NTM species, including, for example, M. bovis, M. africanum, BCG, M. avium, M. intracellulare, M. celatum, M. genavense, M. haemophilum, M. kansasii, M. ulcerans, M. Marinum, M. canitelli, M. abscessus, M. lilandii, M simiae, M. vaccae, M. fortuitum, and M. scrofulaceum species. The fusion polypeptide can be any one of the fusion polypeptides described herein including, for example, a fusion polypeptide that has at least a 90% sequence identity to ID93-1, ID93-2, ID83-1, ID83-2, ID97 or ID91. The fusion polypeptide can be ID93-1, ID93-2, ID83-1, ID83-2, or ID97 or ID91.

In some embodiments, provided herein is a method of preventing a nontuberculous Mycobacterium (NTM) infection in a subject, comprising administering to a subject an effective amount of any one of the fusion polypeptides, or pharmaceutical compositions comprising the fusion polypeptides provided herein. In some embodiments, the subject is Quantiferon positive. In some embodiments, the subject is Quantiferon negative. In any of these embodiments, the NTM infection can be the primary instance of a NTM infection or the second instance of a NTM infection (e.g., a secondary infection). The NTM can be any one of the NTM species, including, for example, M. bovis, M africanum, BCG, M. avium, M. intracellulare, M. celatum, M. genavense, M. haemophilum, M. kansasii, M. ulcerans, M. Marinum, M. canitelli, M. abscessus, M. lilandii, M. simiae, M. vaccae, M. fortuitum, and M. scrofulaceum species. The fusion polypeptide can be any one of the fusion polypeptides described herein including, for example, a fusion polypeptide that has at least a 90% sequence identity to ID93-1, ID93-2, ID83-1, ID83-2, ID97 or ID91. The fusion polypeptide can be ID93-1, ID93-2, ID83-1, ID83-2, or ID97 or ID91.

In some embodiments, provided herein is a method of treating or preventing pulmonary infection caused by infection with Mtb or NTM wherein the lung disease is a result of reactivation of a primary NTM infection, a secondary NTM infection, or a latent NTM infection. In some embodiments, the subject is Quantiferon positive. In some embodiments, the subject is Quantiferon negative. In some embodiments, the subject had previously been treated for a TB infection and does not have active disease (e.g., TB or NTM disease) at the time of treatment. In some embodiments, the subject had previously been treated for a NTM infection and does not have active disease (e.g, TB or NTM disease) at the time of treatment. The NTM can be any one of the NTM species, including, for example, M. bovis, M africanum, BCG, M. avium, M. intracellulare, M. celatum, M. genavense, M. haemophilum, M. kansasii, M. ulcerans, M. Marinum, M. canitelli, M. abscessus, M. lilandii, M. simiae, M. vaccae, M. fortuitum, and M. scrofulaceum species.

In some embodiments, provided herein is a method of reducing a sign or symptom of an active disease (e.g., active pulmonary infection) in a subject, comprising administering to a subject an effective amount of any one of the fusion polypeptides, or pharmaceutical compositions comprising the fusion polypeptides provided herein. The active disease may be associated with a secondary Mtb or NTM infection. The active disease may be associated with a NTM infection. The active disease may be TB and associated with a secondary Mtb infection. In some embodiments, the subject is Quantiferon positive. In some embodiments, the subject is Quantiferon negative.

In some embodiments, an effective amount of any one of the fusion polypeptides, or pharmaceutical compositions comprising the fusion polypeptides provided herein is administered before, simultaneously with, or after the adminstiration of a chemotherapeutic agent.

Kits and Articles of Manufacture

Also contemplated in certain embodiments are kits comprising, for example, the fusion polypeptides, Mtb antigens, NTM antigens, and pharmaceutical compositions provided herein; the polynucleotides encoding the fusion polypeptides, Mtb antigens, and NTM antigens provided herein; and the immunological adjuvants provided herein, which may be provided in one or more containers. In one embodiment all components of the compositions are present together in a single container, but the invention embodiments are not intended to be so limited and also contemplate two or more containers in which, for example, an immunological adjuvant is separate from, and not in contact with, the fusion polypeptide composition component.

The kits of the invention may further comprise instructions for use as herein described or instructions for mixing the materials contained in the vials. In some embodiments, the material in the vial is dry or lyophilized. In some embodiments, the material in the vial is liquid.

A container according to such kit embodiments may be any suitable container, vessel, vial, ampule, tube, cup, box, bottle, flask, jar, dish, well of a single-well or multi-well apparatus, reservoir, tank, or the like, or other device in which the herein disclosed compositions may be placed, stored and/or transported, and accessed to remove the contents. Typically, such a container may be made of a material that is compatible with the intended use and from which recovery of the contained contents can be readily achieved. Non-limiting examples of such containers include glass and/or plastic sealed or re-sealable tubes and ampules, including those having a rubber septum or other sealing means that is compatible with withdrawal of the contents using a needle and syringe. Such containers may, for instance, by made of glass or a chemically compatible plastic or resin, which may be made of, or may be coated with, a material that permits efficient recovery of material from the container and/or protects the material from, e.g., degradative conditions such as ultraviolet light or temperature extremes, or from the introduction of unwanted contaminants including microbial contaminants. The containers are preferably sterile or sterilizeable and made of materials that may be compatible with any carrier, excipient, solvent, vehicle or the like, such as may be used to suspend or dissolve the herein described fusion polypeptides, antigens, and pharmaceutical compositions.

TLR4 Agonists

Provided herein are TLR4 agonists (toll-like receptor 4 agonists) that can be used in the compositions and methods described herein. A TLR4 agonist can comprise a glucopyranosyl lipid adjuvant (GLA), such as those described in U.S. Patent Publication Nos. US2007/021017, US2009/045033, US2010/037466, and US 2010/0310602, the contents of which are incorporated herein by reference in their entireties.

For example, the TLR4 agonist can be a synthetic GLA adjuvant having the following structure of Formula (IV):

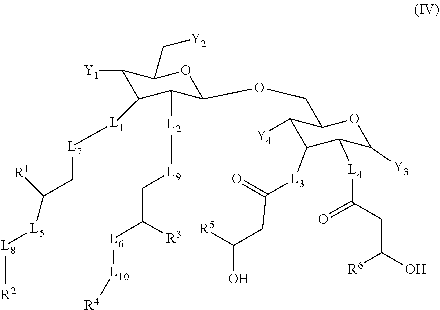

or a pharmaceutically acceptable salt thereof, wherein:

L₁, L₂, L₃, L₄, L₅ and L₆ are the same or different and independently —O—, —NH— or —(CH₂)—;

L₇, L₈, L₉, and L₁₀ are the same or different and independently absent or —C(═O)—;

Y₁ is an acid functional group;

Y₂ and Y₃ are the same or different and independently —OH, —SH, or an acid functional group;

Y₄ is —OH or —SH;

R₁, R₃, R₅ and R₆ are the same or different and independently C₈₋₁₃ alkyl; and R₂ and R₄ are the same or different and independently C₆₋₁₁ alkyl.

In some embodiments of the synthetic GLA structure, R¹, R³, R⁵ and R⁶ are C₁₀ alkyl; and R² and R⁴ are C₈ alkyl. In certain embodiments, R¹, R³, R⁵ and R⁶ are C₁₁ alkyl; and R² and R⁴ are C₉ alkyl.

For example, in certain embodiments, the TLR4 agonist is a synthetic GLA adjuvant having the following structure of Formula (V):

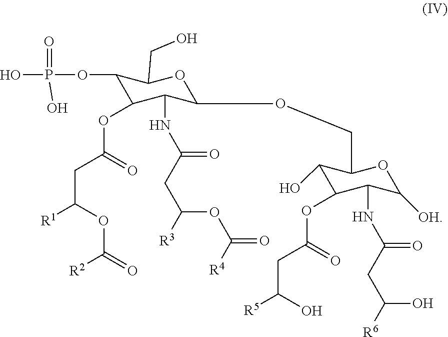

In a specific embodiment, R¹, R³, R⁵ and R⁶ are C₁₁-C₂₀ alkyl; and R² and R⁴ are C₁₂-C₂₀ alkyl.

In another specific embodiment, the GLA has the formula set forth above wherein R¹, R³, R⁵ and R⁶ are C₁₁ alkyl; and R² and R⁴ are C₁₃ alkyl.

In another specific embodiment, the GLA has the formula set forth above wherein R′, R³, R⁵ and R⁶ are C₁₀ alkyl; and R² and R⁴ are C₈ alkyl.

In another specific embodiment, the GLA has the formula set forth above wherein R′, R³, R⁵ and R⁶ are C₁₁-C₂₀ alkyl; and R² and R⁴ are C₉-C₂₀ alkyl. In certain embodiments, R′, R³, R⁵ and R⁶ are C₁₁ alkyl; and R² and R⁴ are C₉ alkyl.

In certain embodiments, the TLR4 agonist is a synthetic GLA adjuvant having the following structure of Formula (V):

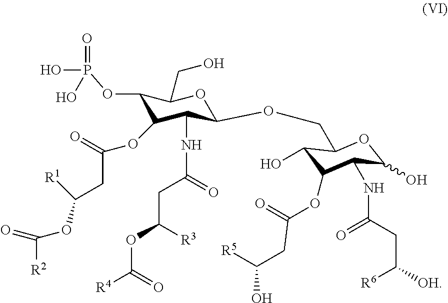

In certain embodiments of the above GLA structure, R′, R³, R⁵ and R⁶ are C₁₁-C₂₀ alkyl; and R² and R⁴ are C₉-C₂₀ alkyl. In certain embodiments, R′, R³, R⁵ and R⁶ are C₁₁ alkyl; and R² and R⁴ are C₉ alkyl.

In certain embodiments, the TLR4 agonist is a synthetic GLA adjuvant having the following structure of Formula (VI):

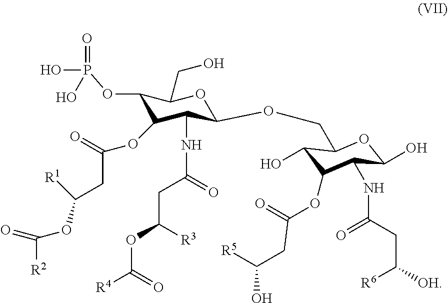

In certain embodiments of the above GLA structure, R′, R³, R⁵ and R⁶ are C₁₁-C₂₀ alkyl; and R² and R⁴ are C₉-C₂₀ alkyl. In certain embodiments, R′, R³, R⁵ and R⁶ are C₁₁ alkyl; and R² and R⁴ are C₉ alkyl.

In certain embodiments, the TLR4 agonist is a synthetic GLA adjuvant having the following structure of Formula (VII):

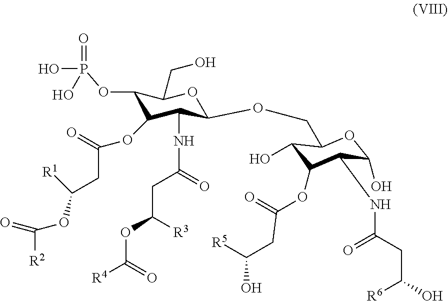

In certain embodiments of the above GLA structure, R¹, R³, R⁵ and R⁶ are C₁₁-C₂₀ alkyl; and R² and R⁴ are C₉-C₂₀ alkyl. In certain embodiments, R³, R⁵ and R⁶ are CH alkyl; and R² and R⁴ are C₉ alkyl.

In certain embodiments, the TLR4 agonist is a synthetic GLA adjuvant having the following structure (SLA):

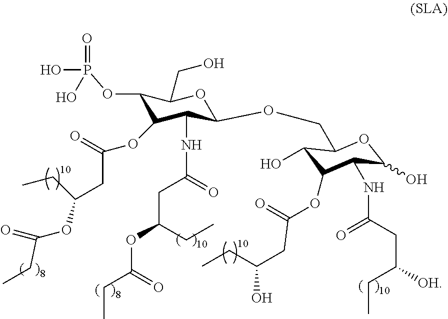

In certain embodiments, the TLR4 agonist is a synthetic GLA adjuvant having the following structure:

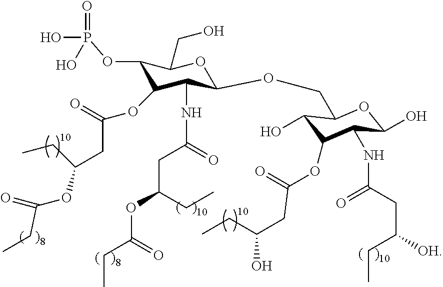

In certain embodiments, the TLR4 agonist is a synthetic GLA adjuvant having the following structure:

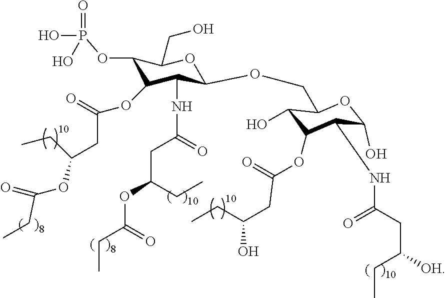

In another embodiment, the TLR4 agonist is an attenuated lipid A derivative (ALD) is incorporated into the compositions described herein. ALDs are lipid A-like molecules that have been altered or constructed so that the molecule displays lesser or different of the adverse effects of lipid A. These adverse effects include pyrogenicity, local Shwarzman reactivity and toxicity as evaluated in the chick embryo 50% lethal dose assay (CELD50). ALDs useful according to the present disclosure include monophosphoryl lipid A (MLA) and 3-deacylated monophosphoryl lipid A (3D-MLA). MLA and 3D-MLA are known and need not be described in detail herein. See for example U.S. Pat. No. 4,436,727 issued Mar. 13, 1984, assigned to Ribi ImmunoChem Research, Inc., which discloses monophosphoryl lipid A and its manufacture. U.S. Pat. No. 4,912,094 and reexamination certificate B1 U.S. Pat. No. 4,912,094 to Myers, et al., also assigned to Ribi ImmunoChem Research, Inc., embodies 3-deacylated monophosphoryl lipid A and a method for its manufacture. Disclosures of each of these patents with respect to MLA and 3D-MLA are incorporated herein by reference.

In the TLR4 agonist compounds above, the overall charge can be determined according to the functional groups in the molecule. For example, a phosphate group can be negatively charged or neutral, depending on the ionization state of the phosphate group.

The TLR4 agonists can be formulated using methods known in the art, for example, as an aqueous nanosuspension, an oil-in-water emulsion, a liposome, and an alum-adsorbed formulation. (See, for example, GLA-AF, GLA-SE, GLA-LS and GLA-Alum in Misquith et al., Colloids Surf B Biointerfaces. 2014 Jan. 1; 113).

Provide herein are methods of preventing or treating a nontuberculous Mycobacterium (NTM) infection in a subject, comprising administering to a subject an effective amount of a TLR4 agonist (i.e., any of the TLR agonists described herein) alone or in combination with any one of the fusion polypeptides described herein. The subject can be Quantiferon positive or negative. In any of these embodiments, the NTM infection can be the primary instance of a NTM infection or the second instance of a NTM infection (e.g., a secondary infection). The NTM can be any one of the NTM species, including, for example, M. bovis, M africanum, BCG, M. avium, M. intracellulare, M. celatum, M. genavense, M. haemophilum, M. kansasii, M. ulcerans, M. Marinum, M. canitelli, M. abscessus, M. lilandii, M. simiae, M. vaccae, M. fortuitum, and M. scrofulaceum species. The fusion polypeptide can be any one of the fusion polypeptides described herein including, for example, a fusion polypeptide that has at least a 90% sequence identity to ID93-1, ID93-2, ID83-1, ID83-2, ID97 or ID91. The fusion polypeptide can be ID93-1, ID93-2, ID83-1, ID83-2, or ID97 or ID91. In exemplary embodiments, the TLR is SLA or GLA having the structure of Formula (IV) wherein R′, R³, R⁵ and R⁶ are C₁₁ alkyl; and R² and R⁴ are C₁₃ alkyl.

Also provided herein are methods of reducing NTM bacterial burden in a subject comprising contacting a cell of the subject with (i) a TLR 4 agonist (i.e., any of the TLR4 agonists described herein), (ii) any of the fusion polyeptides described herein or (iii) a combination thereof. The subject's cell can be in the subject and contacting is via administering the TRL4 agonist and/or any of the fusion polypeptides described herein to the subject. The NTM can be any one of the NTM species, including, for example, M. bovis, M africanum, BCG, M. avium, M. intracellulare, M. celatum, M. genavense, M. haemophilum, M. kansasii, M. ulcerans, M. Marinum, M. canitelli, M. abscessus, M. lilandii, M. simiae, M. vaccae, M. fortuitum, and M. scrofulaceum species. The fusion polypeptide can be any one of the fusion polypeptides described herein including, for example, a fusion polypeptide that has at least a 90% sequence identity to ID93-1, ID93-2, ID83-1, ID83-2, ID97 or ID91. The fusion polypeptide can be ID93-1, ID93-2, ID83-1, ID83-2, or ID97 or ID91. In exemplary embodiments, the TLR is SLA or GLA having the structure of Formula (IV) wherein R¹, R³, R⁵ and R⁶ are C₁₁ alkyl; and R² and R⁴ are C₁₃ alkyl.

Also provided are pharmaceutical compositions comprising a TLR4 agonist as described herein (e.g., formulated GLA) and may further comprise one or more components as provided herein that are selected, for example, from antigen, additional TLR agonist, and co-adjuvant in combination with a pharmaceutically acceptable carrier, excipient or diluent.

Also provided are pharmaceutical compositions comprising a TLR4 agonist as described herein (e.g., formulated GLA) in combination with any of the fusion polypeptides described herein including for example ID93-1, ID93-2, ID83-1, ID83-2, or ID97 or ID91.

General methods of administering TLR4 agonists, including GLA, to a subject for the treatment of disease are known in the art and can be used herein to determine an optimized formulation for the treatment of NTMs in a subject and for reducing bacterial burden in a subject. For example, about 0.001 μg/kg to about 100 mg/kg body weight will generally be administered, typically by the intradermal, subcutaneous, intramuscular or intravenous route, or by other routes. In a more specific embodiment, the dosage is about 0.001 μg/kg to about 1 mg/kg. In another specific embodiment, the dosage is about 0.001 to about 50 μg/kg. In another specific embodiment, the dosage is about 0.001 to about 15 μg/kg. In another specific embodiment, the amount of GLA administered is about 0.01 μg/dose to about 5 mg/dose. In another specific embodiment, the amount of GLA administered is about 0.1 μg/dose to about 1 mg/dose. In another specific embodiment, the amount of GLA administered is about 0.1 μg/dose to about 100 μg/dose. In another specific embodiment, the GLA administered is about 0.1 μg/dose to about 10 μg/dose.

It will be evident to those skilled in the art that the number and frequency of administration will be dependent upon the response of the host. “Pharmaceutically acceptable carriers” for therapeutic use are well known in the pharmaceutical art, and are described, for example, in Remingtons Pharmaceutical Sciences, Mack Publishing Co. (A. R. Gennaro edit. 1985). For example, sterile saline and phosphate-buffered saline at physiological pH may be used. Preservatives, stabilizers, dyes and even flavoring agents may be provided in the pharmaceutical composition. The pharmaceutical compositions may be in any form known in the art which allows for the composition to be administered to a patient. The pharmaceutical composition is formulated so as to allow the active ingredients contained therein to be bioavailable upon administration of the composition to a patient.

The following Examples are offered by way of illustration and not by way of limitation.

### EXAMPLES

GLA used in the examples has the structure of Formula (IV) wherein R¹, R³, R⁵ and R⁶ are C₁₁ alkyl; and R² and R⁴ are C₁₃ alkyl.

### Example 1: Construction of the ID93-2 Expression Vector

The selected Mtb antigens were individually cloned from Mtb HRv37 genomic DNA into the pET-28a vector (Invitrogen) (Bertholet et al., 2008; Identification of human T cell antigens for the development of vaccines against Mycobacterium tuberculosis), using a cloning strategy that produces an N-terminal 6xHis-tag which was utilized for purification of research lots of ID93-2. The cloning primers were designed to introduce appropriate restriction sites to allow directional cloning. The sequences of the primers used for amplifying the four antigens are listed in Table 5.

TABLE 5
Cloning primers for ID93-2
component antigens

| # | Primer Name | Primer Sequence |
|---|---|---|
| 1 | 5′: Rv1813mat- | CAATTACATATGGGTACC |
| | 5NdeI-Kpni | CATCTCGCCAACGGTTCG |
| | | ATG |
| | | (SEQ ID NO: 61) |
| 2 | 3′: Rv1813mat- | CAATTAGAGCTCGTTGCA |
| | 3SacIgo | CGCCCAGTTGACGAT |
| | | (SEQ ID NO: 62) |
| 3 | 5′: Rv3620- | CAATTAGAGCTCATGACC |
| | 5SacI | TCGCGTTTTATGACG |
| | | (SEQ ID NO: 63) |
| 4 | 3′: Rv3620- | CAATTAGTCGACGCTGCT |
| | 3SalIgo | GAGGATCTGCTGGGA |
| | | (SEQ ID NO: 64) |
| 5 | 5′: Rv2608- | CAATTAGTCGACATGAAT |
| | 5 SalI | TTCGCCGTTTTGCCG |
| | | (SEQ ID NO: 65) |
| 6 | 3′: Rv2608- | CAATTAAAGCTTTTAAGT |
| | 3ScaI-HindIII | ACTGAAAAGTCGGGGTAG |
| | | CGCCGG |
| | | (SEQ ID NO: 66) |
| 7 | 5′: Rv3619- | CAATTACATATGACCATC |
| | SNdei | AACTATCAATTC |
| | | (SEQ ID NO: 67) |
| 8 | 3′: Rv3619- | CAATTAGGTACCGGCCCA |
| | 3KpnI | GCTGGAGCCGACGGC |
| | | (SEQ ID NO: 68) |

For clinical production, the entire sequence of ID93-2 was subcloned into the pET-29a vector using a strategy designed for expression without any added amino acid tags. Using standard molecular biological techniques, the ID93-2/pET-29a expression vector was constructed as follows. Rv1813 was PCR amplified from HRv37 genomic DNA, digested with NdeI/SacI, and ligated into the empty pET-28a vector to create the pET-28a/Rv1813 construct. Next, Rv3620 was PCR amplified from HRv37 genomic DNA, digested with SacI/SalI, and ligated into the pET-28a/Rv1813 construct to create the pET-28a/Rv1813/Rv3620 construct. Rv2608 was PCR amplified from HRv37 genomic DNA, digested with SalI/HindIII and ligated into the pET-28a/Rv1813/Rv3620 construct to create the pET-28a/Rv1813/Rv3620/Rv2608 construct. Rv3619 was PCR amplified from HRv37 genomic DNA, digested with NdeI/KpnI, and ligated into the pET-28a/Rv1813/Rv3620/Rv2608 construct to create the pET-28a/Rv1813/Rv3620/Rv2608/Rv3619 construct. The resulting four-antigen fusion construct (ID93-2) was digested with NdeI/HindIII and the ID93-2 sequence was subcloned into the isopropyl-β-D-thiogalactopyranoside (IPTG)-inducible pET-29a expression vector. The pET-29a vector has a T7 promoter and confers kanamycin resistance. The ID93-2 expression construct was confirmed by sequencing and restriction fragment analysis. The ID93-2/pET-29a expression vector was transformed into Escherichia coli (E. coli) HMS174 cells and a Master Cell Bank (MCB) was manufactured.

ID93-2 was produced by standard fermentation according to methods known in the art. The cell culture is harvested and pelleted. The cell pellets are resuspended in Lysis Buffer (25 mM Tris, 5 mM EDTA, pH 8.0) and an M-110Y Microfluidizer® is used to disrupt the cells. The cells are passed through the Microfluidizer two times at a pressure of 15,000-18,000 psi. The suspension is centrifuged at 16,000 g for 2 h. Under these conditions, the inclusion bodies (IB) containing ID93-2 protein are pelleted, while most of the cell debris remains in the supernatant. The ID93-2 protein is purified by column chromatography by binding on an anion exchange column and elutes with DEAE Elution Buffer. The DEAE Sepharose FF eluate is loaded onto another equilibrated anion exchange column Q Sepharose FF anion exchange column. The flow through containing the protein is collected in a single container. 5% Glycerol ammonium sulfate and is added to the Q Sepharose FF flow through (containing ID93 protein) and incubated for 1 h. The protein pool containing glycerol and is loaded onto an equilibrated hydrophobic interaction chromatography column and the column is eluted with Elution Buffer for Phenyl Sepharose HP. β-mercapto-ethanol is added to the eluate pool to a final concentration of 5 mM and incubated for 30 min in order to reduce the protein sample and the pool is diafiltered to 20 mM Tris pH 8.0, the protein concentration is adjusted to 0.5 mg/mL, filter sterilized with a 0.22 μm filter membrane and stored at <65° C.

### Example 2: Clinical Trial of ID93-2 GLA-SE to Assess Whether ID93-2+GLA-SE was Immunogenic Upon Administration to Adults Who have been Vaccinated with BCG and Live in a TB Endemic Region where 80% of Adults are Latently Infected with M. tuberculosis

BCG is the only TB vaccine currently licensed for use in humans and appears to be effective at preventing severe disseminated disease in newborns and young children but fails to protect against pulmonary TB in adults (Andersen P, Doherty T M. The success and failure of BCG—implications for a novel tuberculosis vaccine. Nat Rev Microbiol 2005; 3:656-662). Even though variable efficacy has been shown with BCG vaccination in human trials, BCG is unlikely to be replaced in the near future and is the reference standard to which all other experimental vaccines are compared. A number of countries with a lower incidence of TB, including the United States, have not adopted or have withdrawn from routine BCG vaccination, preferring to screen for and treat TB with antibiotics.

Clinical Trial

A Phase 1b, randomized, double-blind, placebo-controlled, dose-escalation evaluation trail was conducted, with two dose levels of the ID93-2 composition, Cohorts 1, 2, and 3 (10 μg, 2 μg, and 10 μg respectively) were administered intramuscularly (IM) in combination with a 2 μg GLA-SE adjuvant dose at Days 0, 28, and 112. Cohort 4 was immunized IM with 10 μg ID93-2 composition in combination with a 5 μg GLA-SE adjuvant dose at Day 0. This study was conducted in 66 HIV-negative, healthy South African subjects with previous BCG vaccination. The BCG vaccine used to immunize the South African subjects lacked the antigen components RV 3619 and RV 3620 found in the ID93-2 protein. Both QFT-(Cohorts 1-4) (QFT negative as an indication of subjects not latently infected with M. tuberculosis) subjects and QFT+ positive Cohorts 2, 3, 4), participants were enrolled in the study.

Subjects were randomized to placebo or treatment groups at a 3:1 ratio (Cohort 1) or 5:1 ratio (Cohorts 2-4) to receive ID93-2+GLA-SE or saline placebo on Days 0, 28, and 112.

A summary of immunologic assays to be performed on blood specimens is shown in Table 6.

TABLE 6
Summary of Immunology Analysis Performed

| Sample type | Assay | Study Days |
|---|---|---|
| Primary Immunology: | Flow cytometry, | Days 0, 14, 42, 112, |
| Peripheral Blood | Intracellular cytokine | 126, 196, 294 |
| Mononuclear | staining (ICS) | |
| Cells (PBMCs) | | |
| Exploratory | IFN-γ ELISPOT | Days 0, 14, 42, 112, |
| Immunology Cells | | 126, 196, 294 |
| (PBMCs) | | |
| Exploratory | Whole blood ICS | Days 0, 14, 42, 112, |
| Immunology | | 126, 196, 294 |
| Whole Blood | | |
| Exploratory | Antigen-specific IgG | Days 0, 126, 294 |
| Immunology | | |
| Serum | | |
| Exploratory | Autoimmune antibodies | Days 0, 294 |
| Immunology | (ELISA) | |
| Serum | | |
| Exploratory | Microarray transcriptional | Days 0, 1, 3, 7, 126 |
| Immunology | profiling or RNA sequencing | |
| Whole Blood for | | |
| RNA Extraction | | |

Immunological Methods for Analysis of Subject Samples

Methods for short-term whole blood stimulation and cryopreservation. 1 mL of fresh whole blood from each study subject was stimulated within 75 mins of phlebotomy using 1 μg/ml/peptide of pools of Rv1813 (a or b), Rv2608 (a or b), Rv3619, or Rv3620. For each participant and time point, 5 μg/ml PHA was used as a positive control, and an unstimulated tube was used as a negative control. Co-stimulatory antibodies anti-CD28 and anti-CD49d (BD Biosciences, 1 μg/mL) were included in all assay conditions. The whole blood was incubated at 37° C. for 12 hours, and Brefeldin-A (Sigma, 10 μg/mL) was added for the last five hours of incubation. The blood was then harvested with EDTA (Sigma, 2 μM), red blood cells lysed and white cells fixed with FACS lysing solution (BD Biosciences). White cells were pelleted and cryopreserved with 10% DMSO (Sigma) in 40% fetal calf serum (HyClone).

Methods for Intracellular cytokine staining (ICS). Intracellular cytokine staining (ICS) is a widely used flow cytometry based assay that detects expression and accumulation of cytokines within the endoplasmic reticulum of cells that respond to antigenic stimulation. ICS may be used in combination with a variety of antibodies that bind to cytokines and cellular markers to perform in-depth phenotypic and functional analyses of single cells within a complex cell population, such as peripheral blood. In this study, we batched analysis of the cryopreserved, stimulated white cells from each individual for ICS antibody staining after completion of the follow-up study period, to ensure less technical assay variation in outcomes. These analyses of the fixed white blood cells for this study were preceded by an optimization process that evaluated the following: optimal antibody concentrations, optimal antibody-fluorochrome combinations, optimal photomultiplier tube (PMT) voltages, fluorescence minus one (FMO) controls, and optimal gating strategy. The acquisition of the stained cells was performed on a BD LSR II cytometer configured for 4 lasers and 18 detectors.

Stimulated, fixed and frozen white cells from whole blood were thawed in a water bath at 37 μC for a short period. The thawed cells were then transferred to labeled tubes containing phosphate buffered saline (PBS, BioWhittaker) and washed and permeabilised using Perm/Wash solution (BD Biosciences). Cells were then stained with the following anti-human antibodies: CD3-BV421 (UCHT1), CD4-BV786 (SK3), CD8-PerCP-Cy5.5 (SK1), CCR7-PE (150503), CD45RA-BV605 (HI100), CD14-BV650 (M5E2), CD16PE-CF594 (3G8), IFN-g-AF700 (B27), IL-2-FITC (5344.111), IL-17-AF647 (SCPL1362) (BD Biosciences), and TNF-α-PE-Cy7 (MAb11) (eBioscience).

Samples were stained, acquired and analyzed in batch. For every ICS assay experiment, compensation controls (single stained positive and negative mouse kappa compensation beads) were included. These controls were processed in parallel with the study samples during the staining and acquisition process to allow post-acquisition data compensation.

Methods for Flow Data Analysis. Samples were run on the BD LSRII cytometer, data analysis was performed using FlowJo software (v. 9.9, TreeStar). Data files were uploaded to a pre-designed analysis template. Individual gates were adjusted to only include cell populations that were predefined to yield outcomes of interest. The following inclusion/exclusion criteria were applied to determine which data to include in the final analysis:

1. The negative (unstimulated) control had to be present and interpretable for each set of samples from a study day.

2. The PHA-induced (positive control) total cytokine response by CD4+ T cells had to be greater than the median plus three median absolute deviations (3MAD) of the total cytokine response by CD4+ T cells of the negative (unstimulated) controls of all participants in the study.

3. For each sample, the PHA-induced total cytokine response in CD4+ T cells had to be greater than the total cytokine response in CD4+ T cells of its negative (unstimulated) control.

Data analysis and Statistics. Percentage T cell response in the ICS assay using PBMCs was summarized by treatment regimen, T cell type (CD4+ and CD8+), and stimulation antigen (Rv1813 (a or b), Rv2608 (a or b), Rv3619, and Rv3620) using median DMSO-subtracted cytokine/function (CD107a, CD154, IFN-γ, interleukin [IL]-2, IL-17A, IL-22, or tumor necrosis factor [TNF], alone or in any combination [excluding CD107a single positive events]) response and associated 95% confidence intervals (CI) based on order statistics.

ICS responses were also analyzed as follows: the number (percentage) of responders in each treatment regimen, determined using an interim responder definition that was developed by The Statistical Center for HIV/AIDS Research & Prevention (SCHARP) to assess vaccine “take,” herein referred to as the SCHARP method, was summarized by T cell type and stimulation antigen. Pairwise comparisons between treatment regimens for number (percentage) of responders were conducted, using Fisher's Exact test adjusted for multiplicity by means of the Holm method. The SCHARP method for determining responder status for each participant was based on the multiplicity-adjusted (Holm method) Fisher's Exact test on a subset of functions (IFN-g TNFα, IL-2, and/or CD154) which were positive combinations of one or more of these functions, and with baseline responder status taken into account.

Median DMSO-subtracted function responses were compared across treatment regimens based on the Kruskal-Wallis test per visit, to identify any difference among the 4 treatment regimens. If a significant difference was identified, the Wilcoxon-Mann Whitney test for pairwise comparisons between treatment regimens was performed. Wilcoxon-Mann Whitney p-values were adjusted for multiplicity by the Holm method. Results were summarized for positive combinations of one or more of functions IFN-γ, TNF, IL-2, and/or CD154; and for CD154 alone.

Assessment of immune response by the IFN-γ ELISpot assay was based on the number of IFN-γ spot-forming units (SFU) per 106 PBMC in response to stimulation with one of the four antigenic peptide pools (Rv1813, Rv2608, Rv3619, and Rv3620). Median and 95% CIs (with CIs based on order statistics) were used to present DMSO-subtracted antigen-specific results.

IFN-gELISpot responses were analyzed as follows: the number (percentage) of responders in each treatment regimen, determined using the SCHARP method, was summarized by stimulation antigen. Pairwise comparisons between treatment regimens for number (percentage) of responders were conducted, using Fisher's Exact test adjusted for multiplicity by means of the Holm method. The SCHARP method for determining responder status for each participant was based on the multiplicity-adjusted (Holm method) Fisher's Exact test, with baseline responder status taken into account.

IgG antibody ELISA data were presented as geometric mean of the endpoint titers (log 10) with 95% CI, and mean fold change from baseline presented as the anti-log of (endpoint titer [log 10] result at post-injection visit—endpoint titer [log 10] result at baseline).

Flow cytometric analysis of specific cytokine-expressing T cells was reported after subtraction of the frequencies of cytokine-expressing T cells in the negative control, i.e., blood incubated with co-stimulatory antibodies alone, from the frequencies of cytokine expression in the Rv1813-, Rv2608-, Rv3619-, RV3620-peptide pools, and the PHA stimulated sample. Where comparative analyses involved more than 2 groups and several time points, we either used the Kruskal-Wallis (between groups) or Friedman (within a group) tests. If significance was shown from these tests, we conducted a post-test analysis with Mann-Whitney U (between groups) or Wilcoxon matched paired tests (within a group). In all statistical tests, a p-value of <0.05 was considered significant.

### Example 3: Diverse Functional Differentiation Profiles of ID93-2-Specific CD4 T Cell Responses in Both QFT− and QFT+ Participants Post ID93-2+GLA-SE Vaccination: Both Strong Central Memory and Strong Effector Memory T Cell Antigens in a Fusion Protein

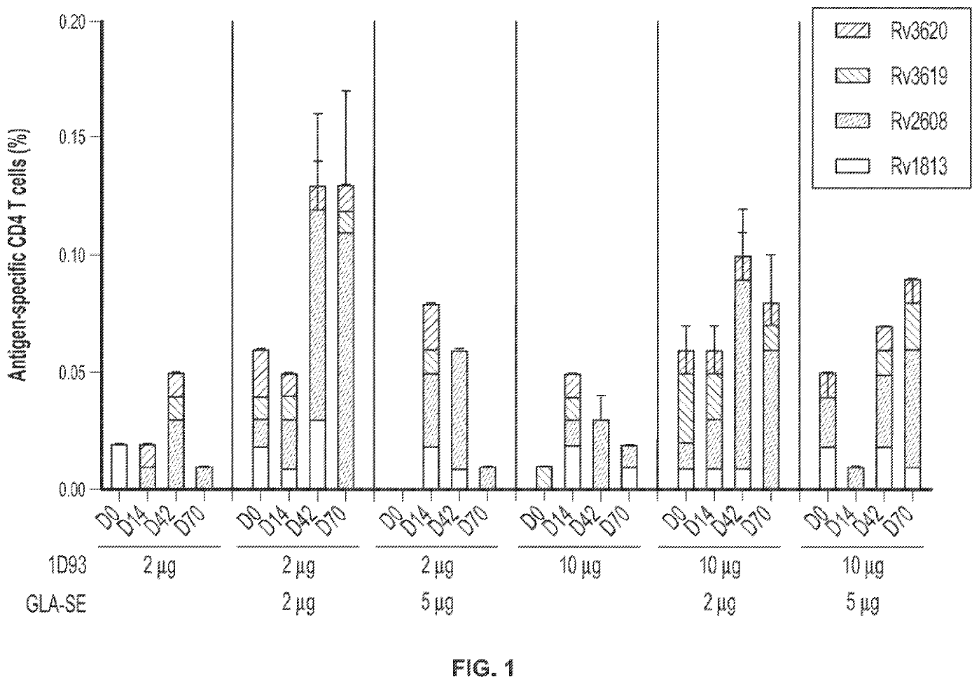

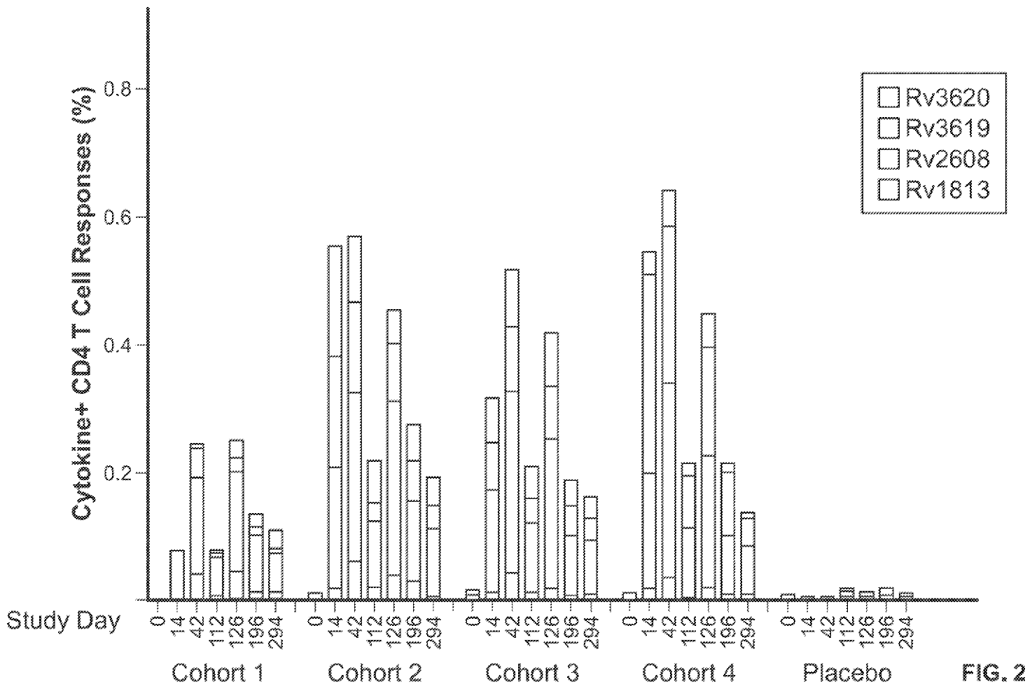

FIG. 1 shows the % of ID93-specific CD4+ T cells (TH1 cells) specific for each individual antigen component of ID93. In this study different doses of ID93 or ID93+GLA-SE were administered on days 0, 28, and 56. Peripheral bood monocytes were collected two weeks after each injection and were stimulated with the antigen subunits comprising ID93: Rv2608 (Rv2608-a or Rv2608-b, all examples), Rv1813, Rv3619, or Rv3620 (FIG. 1). CD4+ T cells are analyzed for the ability to secrete any Th1 cytokine (TNF-α, IFNγ, IL-2, IL-17) using the ICS assay and the panel tested as listed in Example 2. The data indicate that the vaccine is immunogenic, eliciting the desired Th1-type response, and that responses are higher when GLA-SE is included. The data in FIG. 2 analyzed the immune response of after vaccination against each antigenic component of the ID93-2 fusion polypeptide in the ICS assay performed as described in Example 2. The data is presented as stacked bar graphs with the % CD4+ Tells that express any one of the following markers CD3, CD4, CD8, CCR7, CD45RA, CD14-, CD16, and are positive by ICS for Th1 cytokine (TNF-α, IFNγ, IL-2, IL-17). Each bar represents the median total CD4+ T cell response of whole blood to stimulation with pools containing Rv1813-, Rv2608-, Rv3619- or Rv3620-peptides. Error bars represent inter-quartile ranges (IQR) for each stimulation. Vaccinate and placebo recipients are stratified by Cohort, and responses stratified longitudinally by study day. Cohort 1 was comprised of QFT-individuals only and the other Cohorts of predominantyly QFT+ indivduals. Background values (unstimulated) were subtracted. The stacked bars are depicted (top to bottom) as cytokine responders when stimulated with either Rv3620 (uppermost or top box), then sequentially Rv3619, Rv2608, and Rv1813 (bottom bar). The data demonstrates that the peak median measured total CD4 Th1 cytokine response to all antigens, as a cumulative component of their individual parts, was seen at Day 42 in all vaccinated groups. However, the peak response to each of the four individual antigens varied across the cohorts, with responses to Rv3619 in Cohort 2 and Cohort 4 highest at Day 14, and responses to Rv3620 in Cohort 2 highest at Day 14. In Cohorts 2, 3 and 4, a particularly robust CD4 T cell response was seen against Rv2608, followed by near equivalent responses to Rv3619 and Rv3620. In Cohort 1, CD4 T cell responses to Rv2608 at all post vaccination time points was of lower magnitude but not statistically different from the responses in Cohort 3, an ID93-2 and GLA-SE dose matched group of predominantly QFT+ individuals (Mann-Whitney p values at Day 14, Day 42 and Day 126 were 0.3472, 0.2152 and 0.8078, respectively). However, in Cohort 1, the Quantiferon negative group, the Rv3620 and Rv3619 responses (uppermost bar and second from the top respectively) were generally, regardless of number of administrations given and only modest. In addition, CD4 T cell responses were the lowest when stimulated with Rv1813 (either Rv1813-a or Rv1813-b, in all examples) (bottom or lowest of the stacked bars), irrespective of group. No statistically significant CD4 T cell responses to ID93-2-specific antigens were seen post-administration in the placebo vaccinated participants. The vaccine is capable of boosting immune responses in infected individuals to higher levels.

Vaccine-induced responses were also analyzed from PBMCs. Antigen-specific CD4+ DMSO-subtracted ICS responses (i.e., cells expressing CD107a, CD154, IFN-γ, IL-2, IL-17A, IL-22, or TNF alone or in any combination [excluding CD107a single positive events]) were seen in all three ID93-2+GLA-SE regimens, with peak median responses at Study Day 42 (14 days after the second injection). The strongest median response at Study Day 42 across all four vaccine antigens was seen in the ID93-2 2 μg+GLA-SE 2 μg dose (0.278% total response for any cytokine). CD4+ antigen-specific responses were detected 6 months after the final study injection (Study Day 294), with median response across all four vaccine antigens again highest in the ID93-2 2 μg+GLA-SE 2 μg dose (0.148% total response for any cytokine). Rv2608 was the most immunodominant antigen, followed by Rv3619 and Rv3620 for which similar responses were seen; responses to Rv1813 were generally lower. Whole blood ICS assay results were generally consistent with these ICS assay results using PBMCs except that median response magnitudes were higher in the whole blood assay. In addition, the whole blood ICS assay results revealed a robust, durable, and multi-functional CD4 T cell response. The results from this assay also provided evidence that prior Mtb sensitization through natural infection, as measured by QFT, alters the kinetics, magnitude, and quality of the CD4 T cell response to individual antigens in the ID93-2 vaccine.

Statistically significantly different CD4+ overall responder rates (which include participants who were considered a responder for at least one of the four vaccine antigens, based on the SCHARP method) compared to placebo were seen at all time points in the ID93-2+GLA-SE vaccinated groups: 93.3% (2 μg ID93-2+2 μg GLA-SE), 100% (10 μg ID93-2+2 μg GLA-SE), and 93.3% (10 μg ID93-2+5 μg GLA-SE), vs. 41.7% in the placebo dose. Generally, there were no statistically significant differences in CD4+ responder rates for pairwise comparison among the three different ID93-2+GLA-SE dosages at any time point for individual antigens. The highest median CD4+(IFN-g, TNFα, IL-2, and/or CD154) responses on Study Days 42 and 294 were in the ID93-2 2+GLA-SE 2 μg dose, for antigen Rv2608 (0.1259% and 0.0496%, respectively). Statistically significantly higher (based on the Wilcoxon-Mann Whitney test) median CD4+(IFN-g, TNFα, IL-2, and/or CD154) responses compared to placebo were seen more frequently for the ID93-2 2+GLA-SE 2 μg doses than for the other two ID93-2+GLA-SE doses. For pairwise comparisons among ID93-2+GLA-SE doses, statistically significantly higher median CD4+(IFN-g, TNFα, IL-2, and/or CD154) responses were seen only for the ID93-2, 2+GLA-SE 2 μg dose. The analysis of median CD4+(CD154 alone) responses showed similar trends to those for the CD4+(IFN-g, TNF, IL-2, and/or CD154) responses.

Next the antigen-specific CD4+ DMSO-subtracted ICS data were compared to data from the IFN-γ DMSO-subtracted ELISpot. IFN-γ DMSO-subtracted ELISpot responses were seen in all three ID93-2+GLA-SE doses, with the peak median response across all four vaccine antigens at Study Day 42 in the ID93-2, 2+GLA-SE 2 μg dose (1156.7 cells/106 PBMC). IFN-g ELISpot responses were detected 6 months after the final study injection (Study Day 294), with median response across all four vaccine antigens highest in the ID93-2, 10 μg+GLA-SE 5 dose (830 cells/106 PBMC). The strongest responses were to antigens Rv2608, Rv3619 and Rv3620; responses to Rv1813 were minimal. Overall responder rates (which include participants who were considered a responder for at least one of the four vaccine antigens at any time point in the ID93-2+GLA-SE doses were not statistically significantly different compared to placebo (92.9% [2 μg ID93-2+2 μg GLA-SE], 91.3% [10 μg ID93-2+2 μg GLA-SE], and 100.0% [10 ID93-2+5 μg GLA-SE], vs. 75.0% in the placebo dose). Comparison of QFT+ to QFT-responses demonstrated a trend toward stronger median IFN-γ ELISpot responses in QFT+(positive) vs. QFT-(negative) subjects in the ID93-2+10 μg+GLA-SE 2 μg dose. The 10 μg ID93-2+5 μg GLA-SE and 2 μg ID93-2+2 μg GLA-SE doses had a disproportionate higher number of QFT+ subjects so statistical analysis was not meaningful, but the same pattern was demonstrated. In addition, the whole blood ICS assay showed markedly elevated CD4+ T cell responses after a single vaccination with ID93-2+GLA-SE in QFT positive vs. QFT negative participants, reflecting the ability of ID93-2+GLA-SE to elicit an immune response in patients with previous tuberculosis disease. ID93-2+GLA-SE did not induce high numbers specific CD4+ T cell responses to Rv3619 or Rv3620 in QFT-Cohort 1 subjects, that had not been previously infected with M. tuberculosis, compared to placebo, suggesting that for these antigens, the vaccine may be particularly good in boosting immune responses in subjects previously infected with tuberculosis.

Together the data demonstrate that prior tuberculosis sensitization through natural infection, as measured by QFT positivity, alters the kinetics, magnitude, and quality of the CD4 T cell immune response, and that the ID93-2 vaccine demonstrates strong immune reactivity in both tuberculosis naive and infected subjects. Interestingly one of the subjects during the study changed QFT status during the study from positive to negative.

Because the intracellular expression of IFN-γ correlated with the level of differentiation measured by CCR7 and CD45RA in this study, we sought to develop a simple measure of the degree of T cell differentiation into central memory cells and effector memory cells. Among antigen-specific Th1 cells, the pattern of IFNg, TNF-α and IL-2 expression evolves during T cell differentiation from early central memory cells, through effector memory and towards terminally differentiated effector cells (Nat Rev Immunol. 2008 April; 8(4):247-58, T-cell quality in memory and protection: implications for vaccine design. Seder RA1, Darrah P A, Roederer M). Based on these principles, a “functional differentiation score” (FDS) was calculated as the ratio of the proportion of IFNγ+ expressing CD4+ T cells over the proportion of CD4+ T cells not expressing IFNγ (IFNγ-; i.e., expressing TNF-α and/or IL-2).

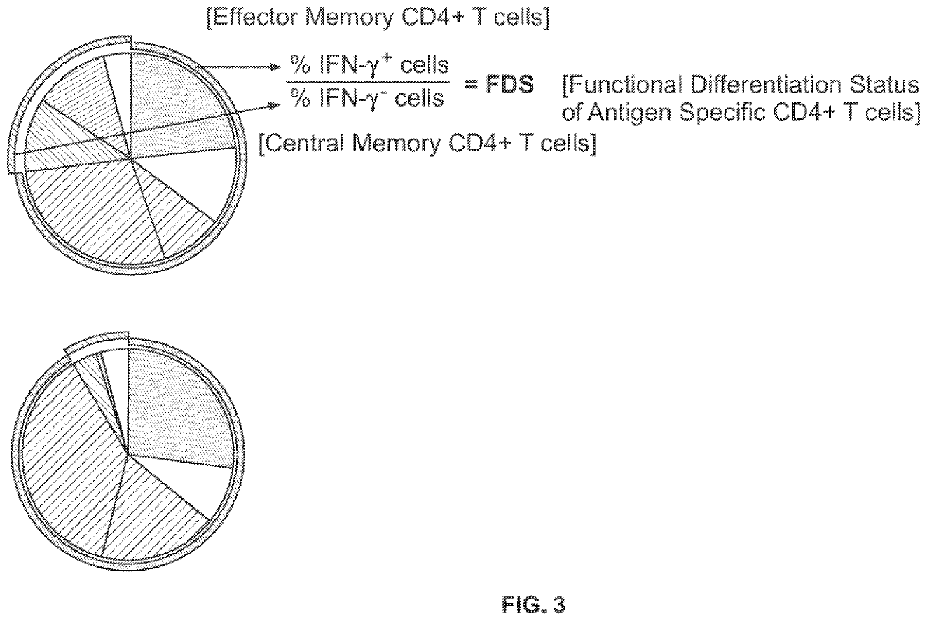

FIG. 3 depicts the general method of analyzing ICS data by FDS. The individual segment of the pie chart represents CD4+ T cells that express various other markers that can be grouped additionally by their IFNγ status (the encircled bolded line). The FDS score then is simply calculated as the percentage of IFN-g+ cells divided by the percentage of IFNγ cells A low FDS score (1 or less) represents cells in the early stages of T cell differentiation, strong central memory populations, whereas a high FDS score (>3) indicates greater differentiation into a strong effector memory population. FDS scores of >1 but <3 represent those cells that have an intermediate phenotype. Previous studies had sought to evaluate whether the FDS score could be used to evaluate the immune response to novel fusion proteins, but to date, no studies have been published regarding the contribution of individual subunit proteins of fusion antigens. (J Immunol Methods. 2004 August; 291(1-2):185-95. Novel application of a whole blood intracellular cytokine detection assay to quantitate specific T-cell frequency in field studies. Hanekom W A1, Hughes J, Mavinkurve M, Mendillo M, Watkins M, Gamieldien H, Gelderbloem S J, Sidibana M, Mansoor N, Davids V, Murray R A, Hawkridge A, Haslett P A, Ress S, Hussey G D, Kaplan G)1 (J Immunol Methods. 2015 February; 417:22-33. Qualification of a whole blood intracellular cytokine staining assay to measure mycobacteria-specific CD4 and CD8 T cell immunity by flow cytometry. Kagina B M1, Mansoor, Kpamegan, Penn-Nicholson, Nemes, Smit, Gelderbloem, Soares, Abel, Keyser, Sidibana, Hughes, Kaplan, Hussey, Hanekom, Scriba).

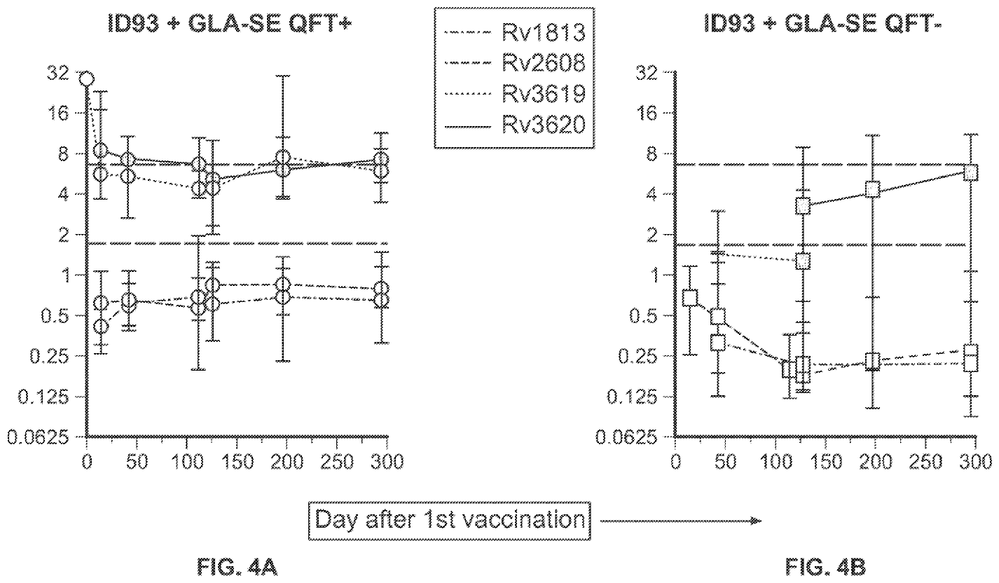

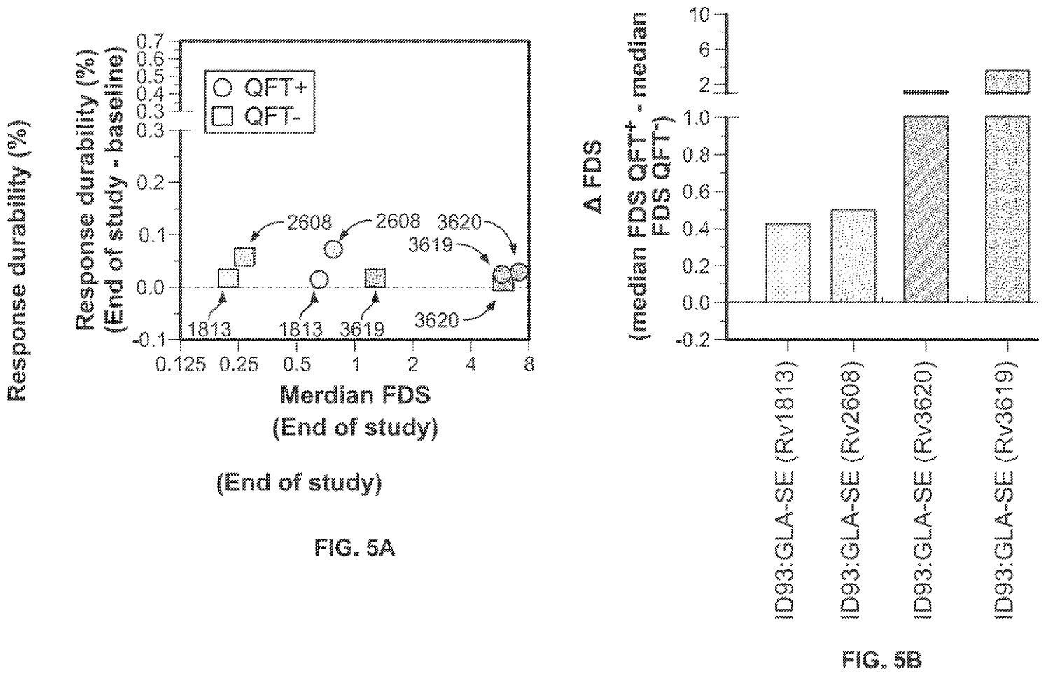

The FDS analysis can be used to analyze the qualitative changes in CD4+ T cell profile status over time by analyzing any change in the FDS score post immunization (FIG. 4 line graph) compared to the baseline response and to evaluate the overall phenotype analysis of response of CD4+ T cell populations to a given antigenic determinant in various populations (FIG. 5A) or in general to any antigenic determinant (FIG. 5B). FIG. 4 presents a line graph of the qualitative analysis of the immune response data from FDS analysis of the cytokine co-expression data for each antigen (Rv1813, Rv2608, Rv3619, and Rv3620) of the ID93-2 fusion protein segregated for the QFT− and QFT+ vaccinated subjects in Cohorts 1 and Cohorts 3 (each group receiving 10 μg of ID93-2+2 μg GLA-SE) over the term of the study including 6 months post the final vaccination. Overall, the data demonstrate that qualitatively, CD4 T cells specific for Rv2608 or Rv1813 can be classified as strong central memory CD4+ T cells (FDS scores of 1 or less) post vaccination, irrespective of baseline QFT status (QFT+ or QFT−) (FIG. 4 and FIG. 5B). While quantitatively, the percentage of CD4+ T cells detected by stimulation with the ID93-2 subunit peptide antigens Rv3619 and Rv3620 was low in ID93-2+GLA-SE vaccinated QFT− subjects, the Rv3620 CD4+ T cell population has more differentiated strong effector memory T cell response profile to this antigen subunit in both previously uninfected or naive tuberculosis subjects (QFT−) and previously infected QFT+ subjects (FIG. 5A, compare the squares (QFT−) to circles (QFT+). In contrast, the Rv3619 CD4+ T cell population demonstrates more of a central memory T cell response profile response to this antigen subunit in uninfected or naive tuberculosis subjects (QFT−) while for previously infected QFT+ subjects (FIG. 5A, compare the squares (QFT−) to circles (QFT+)) the response drives differentiation into a strong effector memory population.

The data demonstrate that underlying M. tuberculosis infection may drive differentiation of Rv3619- and Rv3620-specific CD4 T cells to a greater degree than Rv1813- and Rv2608-specific CD4 T cells. This more effector-like phenotype was maintained post vaccination and at 6 months post the last administration of ID93-2+GLA-SE, suggesting that vaccination did not markedly modulate an already well differentiated Rv3619- and Rv3620-specific CD4 T cell response induced by natural infection with tuberculosis. The data in FIG. 5B demonstrates that for the ID93-2 subunit antigens Rv2608 and Rv1813 that in both subjects previously infected with tuberculosis (QFT+) or tuberculosis naive subjects (QFT−) the qualitative immune response to these antigens is that of a strong central memory response. In QFT+ subjects, immunization with ID93-2 does not significantly change the over profile of strong central memory with either each subsequent vaccination or over time. However, in tuberculosis naive individuals there is, post immunization, a trend toward maturation of CD4 T cells to a strong central memory effector phenotype after immunization with the ID93-2 fusion polypeptide (compare QFT− FIG. 4 B and squares to circles in FIG. 5A). Overall, we saw diversity in the differentiation of ID93-2+GLA-SE induced CD4 T cell responses against each antigen in both subjects with and without underlying M. tuberculosis infection.

### Example 4. Prophylactic Efficacy of ID91 and ID93-2 Vaccines (and Adjuvant Formulations) Against M. avium

The ID91 fusion protein, containing sequence of Rv3619-Rv2389-Rv3478-Rv1886), has been shown to protect mice against M. tuberculosis (Orr M T, Ireton G C, Beebe E A, Huang P W, Reese V A, Argilla D, Coler R N, Reed S G. 2014. Immune subdominant antigens as vaccine candidates against Mycobacterium tuberculosis. J Immunol 193: 2911-8).

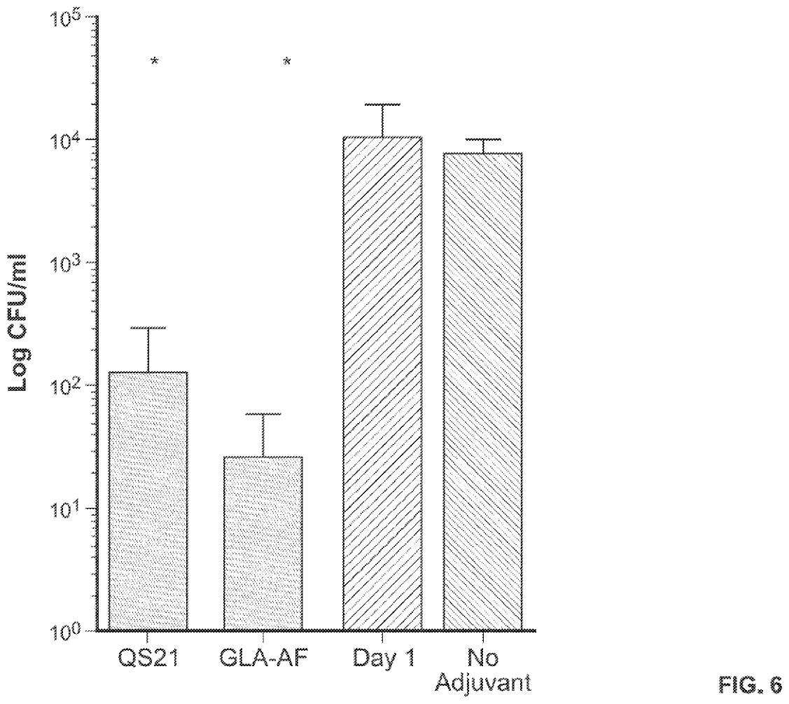

In vitro screen of adjuvants for growth inhibition of NTMs, M. avium, THP-1 cells were differentiated into macrophages overnight by treatment with 100 μg/ml of PMA (Calbiochem). Differentiated macrophages were infected with M. avium at an MOI of 5 for 24 hours (source M. avium). Infected macrophages were treated as indicated for three days with pattern recognition receptor agonists. The data presented in FIG. 6 demonstrates that 24 hours after incubation with saponin (QS21) and GLA-AF, the growth of the M. avium was inhibited. Other TLR agonists, (e.g., SLA-AF) also demonstrated growth inhibition of M. avium (data not shown).

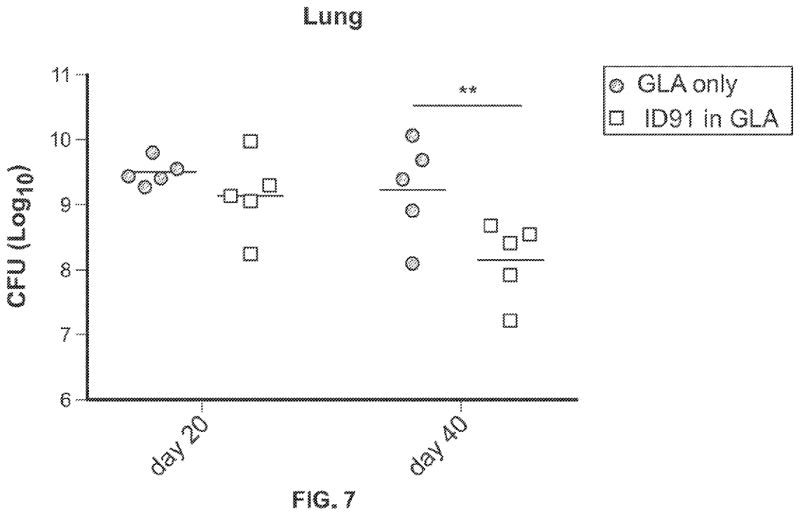

ID91 in combination with GLA-SE and GLA-SE alone were screened in C₅₇BL/6 mice. C57BL/6 mice were immunized 3 times, 3 weeks apart with either GLA-SE or ID91+GLA-SE (i.m). Mice were given an aersol challenge with 1×10⁸ CFU by aerosol M. avium. FIG. 7 shows cfu (Log 10) in the lung either 20 or 40 days post infection. Asteriks represent significance **p<0.05.

Table 7 below shows consensus sequences for NTM with the mycobacterial antigens used in the fusion polypeptides of the present invention.

| | Rv3619 | Rv2389 | Rv3478 | Rv1886 | Rv1813 | Rv3620 | Rv2608 | Rv2875 |
|---|---|---|---|---|---|---|---|---|
| M. tuber- | 100 | 100 | 100 | 100 | 100 | 100 | 100 | 100 |
| culosis | | | | | | | | |
| M. bovis | 99 | 99 | 99 | 99 | 95 | 99 | 99 | 100 |
| M. bovis | 99 | 99 | 99 | 99 | — | — | — | — |
| BCG | | | | | | | | |
| M. | 86 | — | — | — | — | — | — | — |
| ulcerans | | | | | | | | |
| M. infra- | 86 | — | 42 | — | — | — | — | — |
| cellulare | | | | | | | | |
| M. avium | 87 | 61 | 46 | 86 | 81 | 87 | — | — |
| M. kansasii | 87 | 70 | 66 | 90 | 86 | 89 | 58 | 74 |
| M. | 87 | 56 | 53 | 89 | 87 | 91 | 51 | 79 |
| marinum | | | | | | | | |
| M. canettii | 99 | 97 | 98 | 71 | — | — | 99 | 99 |
| M. | — | 48 | — | — | — | — | — | — |
| abscessus | | | | | | | | |
| M. lilandii | 87 | 58 | 53 | 81 | 78 | — | 45 | — |

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method of inducing an immune response in a subject previously vaccinated with BCG and/or with a latent M. tuberculosis infection comprising administering to the subject an effective amount of a fusion polypeptide comprising at least two Mycobacterial antigens, wherein one of the Mycobacterial antigens is an effector memory antigen with a functional differentiation score (FDS) of greater than 3.

2. The method of claim 1, wherein the effector memory antigen is Rv3619 or Rv3620.

3. The method of claim 2, wherein the fusion polypeptide comprises Mycobacterial antigens that have a sequence with at least 90% sequence identity to Rv3619 and Rv3620.

4. The method of claim 1, wherein the central memory antigen is Rv1813 or Rv2608.

5. The method of claim 4, wherein the fusion polypeptide comprises Mycobacterial antigens that have a sequence with at least 90% sequence identity to Rv1813 and Rv2608.

6. The method of claim 2, wherein the fusion polypeptide comprises a Mycobacterial antigen that has a sequence with at least 90% sequence identity to Rv1813, Rv2608, Rv2389, or Rv1886.

7. The method of claim 2, wherein the fusion polypeptide has at least a 90% sequence identity to ID58, ID69, ID71, ID83-1, ID83-2, ID91, ID93-1, ID93-2, ID94-1, ID94-2, ID95, ID97, ID114, ID120-1, ID120-2, ID125-1, or ID125-2.

8. The method of claim 7, wherein the fusion polypeptide has at least a 90% sequence identity to ID93-1 or ID93-2.

9. The method of claim 1, wherein the fusion polypeptide is administered as a pharmaceutical composition comprising an adjuvant.

10. The method of claim 9, wherein the adjuvant is a TLR 4 agonist.

11. The method of claim 10, wherein the TLR 4 agonist is glucopyranosyl lipid A (GLA).

12. The method of claim 10, wherein the TLR 4 agonist is SLA.

13. The method of claim 1, wherein the fusion polypeptide is administered twice.

14. The method of claim 13, wherein the fusion polypeptide is administered twice about 28 days apart.

15. The method of claim 1, wherein the BCG vaccine lacks antigen components Rv3619 and Rv3620.

16. The method of claim 1, wherein the subject was previously vaccinated with BCG.

17. The method of claim 1, wherein the subject has a latent M. tuberculosis infection.

18. The method of claim 1, wherein the subject is Quantiferon positive.

19. The method of claim 1, wherein the immune response comprises a strong Mycobacterial effector memory T cell response.

20. The method of claim 6, wherein the immune response comprises a strong Mycobacterial effector memory T cell response and a strong Mycobacterial central memory T cell response.

21. A kit for performing the method of claim 1.
